(19) United States
(12) Patent Application Publication
(10) Pub. No.: US 2026/0088130 A1
(43) Pub. Date: Mar. 26, 2026

(54) RNA RECOGNITION AND EDITING CODE OF PENTATRICOPEPTIDE REPEAT PROTEINS

(71) Applicant: THE TRUSTEES OF COLUMBIA UNIVERSITY IN THE CITY OF NEW YORK, New York, NY (US)

(72) Inventor: Chaolin Zhang, Scarsdale, NY (US)

(21) Appl. No.: 19/401,790

(22) Filed: Nov. 26, 2025

Related U.S. Application Data
(63) Continuation of application No. PCT/US2024/031309, filed on May 28, 2024.
(60) Provisional application No. 63/504,706, filed on May 26, 2023.

(51) Int. Cl. G16B 30/20; G16B 30/10; G16B 40/20

(52) U.S. Cl. CPC G16B 30/20; G16B 30/10; G16B 40/20

(57) ABSTRACT

Disclosed herein is a computational algorithm that statistically infer a PPR code (preference of a PPR motif for a nucleotide base) while matching PPR proteins with their targets accurately without requiring experimental pairing information. From comprehensive lists of PLS-type PPR proteins and editing sites from more than 1500 PPRs, the algorithm derived a quantitative code including novel amino acid combinations in key positions that confer high specificity. For example, the predicted targets suggests that the recently identified DYW:KP domain is unequivocally responsible for the poorly characterized reverse U-to-C editing.

### RELATED APPLICATIONS

The present application is a Continuation application of International Application No. PCT/US2024/031309, filed on May 28, 2024, which claims the benefit of U.S. provisional patent application 63/504,706, filed May 26, 2023, the entirety of the disclosure of which is hereby incorporated by this reference.

### STATEMENT REGARDING FEDERALLY SPONSORED RESEARCH OR DEVELOPMENT

This invention was made with government support under GM145279 awarded by the National Institutes of Health. The government has certain rights in the invention.

### TECHNICAL FIELD

Disclosed herein is a method of inferring the pentatricopeptide repeat (PPR) code while matching PPR proteins with their targets without requiring experimental pairing information.

### BACKGROUND

Interactions between RNA-binding proteins (RBPs) and their target transcripts are central for co- and posttranscriptional gene regulation (1), and the ability to manipulate such interactions can open up therapeutic opportunities for a range of genetic diseases (2). Most RNA-binding domains, such as RNA-recognition motifs (RRMs) and hnRNP K homology (KH) domains, can adopt varying conformations and protein-RNA interaction interfaces, resulting in recognition of a wide range of short and degenerate RNA sequence elements (3). Accurate prediction of RBP binding specificity from protein sequences is thus a goal yet to be achieved. There are a few notable exceptions. The Pumilio/feminization (PUF) family of proteins in animals has eight Pumilio RNA binding motifs arranged in a repeated array, which recognizes an 8-9 nucleotide (nt) motif with one repeat-one base correspondence (4).

Another extraordinary example is pentatricopeptide repeat (PPR) proteins, which contains an array of between 2 and 30 repeats of a degenerate motif having ˜35 amino acids in length, for modular RNA-binding with one repeat recognizing one nucleotide base (5-7). As used herein, the term “PPR motif” refers to the degenerate motif having ˜35 amino acids in length that is repeated in PPR proteins. PPR proteins are present in most eukaryotes, including humans, but they are dramatically expanded in the land plants. Most plants have several hundreds of PPR proteins, but in certain species, including hornworts, ferns, and some lycophytes, they can have >1500 family members, making them one of the largest gene families accounting for ˜10% of all protein-coding genes (8, 9).

In plants, PPR proteins are almost exclusively localized to the organelles including mitochondria and chloroplast, and they regulate various steps of RNA metabolism essential for the organelle biogenesis (5, 10). Loss of function PPR mutants frequently result in severe developmental or even lethal phenotypes (5, 10). At the molecular level, there are two classes of PPR proteins, P and PLS (10). The P-type PPR proteins consist of entirely the classical P-type PPR motif, and they bind RNA and function as RNA regulators by steric hindrance, such as protecting mRNA termini from degradation by exonucleases (11). The other class, PLS-type PPR proteins, consists of the P-type PPR motif as well as its long (L), and short (L) variants in their PPR arrays (10). These motifs are arranged in PLS triplets in the protein, frequently followed by additional extension domains (E1 and E2) and a catalytic DYW domain (10). The PLS-type PPRs are mostly known as cytosine-to-uridine (C-to-U) RNA editors, or more recently, uridine-to-cytosine (U-to-C) RNA editors (5, 12). In plants, the PLS-type PPRs are known to be involved in RNA editing.

Most PPRs are known to have only one or a few endogenous targets in the organellar transcriptome, owing to the unusual binding specificity of the PPR array in each protein, as dictated by a “PPR code” revealed by extensive biochemical, structural, and bioinformatic analyses. The PPR motif folds into a helix-turn-helix conformation, and when arranged in a repetitive array, forms a superhelical RNA interaction surface that runs in parallel with the bound RNA to dictate one repeat vs. one nucleotide base interaction (11, 13, 14). A few amino acids, especially the ones at the 2nd and last positions (pos. 2 and L), are critical for binding specificity through hydrogen bonding (14-17), and amino acid combinations showing relatively high preferences for each nucleotide base have been identified (e.g., [T/S]N:A, NS:C, TD:G, ND:U) (18-20). The amino acid at position 2 also interacts with RNA directly by buttressing the hydrogen bonds and sandwiching nucleotide base together with the amino acid of the next repeat at the same position (15, 16). This modular RNA recognition mechanism, together with the evolutionary plasticity of PPR arrays, results in a diverse range of unrelated sequences recognized by natural PPRs. This remarkable feature has made PPR proteins attractive candidates for designing engineered proteins (“designer” PPRs) with desired sequence specificities for agricultural and biotechnological applications (15, 21-26). The success of this approach relies on the capability to design proteins with high binding specificity to minimize off-target effects, which have been observed when natural PPRs were expressed in human cells (27).

Bioinformatics analyses that infer the PPR code from experimentally determined regulator-target pairs (18-20) have provided unequivocal confirmation of modular PPR-RNA interaction, prioritized candidate PPR targets, and more recently, suggested a variant DYW domain as a potential candidate for U-to-C editing (9). However, the prediction accuracy is more limited due to the limited number of validated PPR targets, the incomplete understanding of the PPR code, and the qualitative nature of the current code. Accordingly, an improved method is needed for identifying the RNA sequences that are the targets of PPRs. Having improved understanding of the PPR code enables the use of PPRs for modifying gene expression.

### SUMMARY

Disclosed herein in a method for matching pentatricopeptide repeat (PPR) proteins with target sequences in an organism and inferring a PPR code. In some aspects, the PPR code can be inferred without any experimental evidence of PPR-target sequence pairing. The PPR protein may comprise a plurality of a single type of PPR motifs or a plurality of different types of PPR motifs. For example, the PPR protein comprises plurality of different types of PPR motifs selected from the group consisting of: P1, P2, L1, L2, S1, S2, and SS, for example a PLS-type PPR protein.

In some implementations, the method for matching PPR proteins with target sequences in an organism and inferring a PPR code comprises receiving input data points related to PPR editing sites in the organism and at least one PPR protein expressed in the organism; estimating a background base composition for a PPR code from flanking 46-nucleotide sequences upstream of the editing sites (positions −49 to −4); and assigning an initial nucleotide base preference for each PPR codon of the at least one PPR protein based on nucleotide probability parameters to determine an initial PPR code predictive model. The method next comprises calculating an initial scoring matrix for the initial PPR code predictive model and updating the initial PPR code predictive model. The step of updating the initial PPR code predictive model comprises using the initial scoring matrix to score each target sequence with respect to the at least one PPR protein; assigning each target sequence to the at least one PPR protein with a probability; estimating a total number of target sequences assigned to the at least one PPR protein; estimating a total number of each nucleotide base assigned to each PPR codon of the at least one PPR protein based on the estimated total number of target sequences assigned to the at least one PPR protein; and then updating the nucleotide probability parameters based on the estimated total number of each nucleotide base assigned to each PPR codon. After updating the initial PPR code predictive model, the method next comprises assigning an updated nucleotide base preference for each PPR codon based on the updated nucleotide probability parameters to determine an updated PPR code predictive model; calculating an updated scoring matrix for the updated PPR code predictive model; and iteratively updating the updated PPR code predictive model until a best match of a target sequence to the at least one PPR protein does not change any more, indicating a match between the at least one PPR protein and the target sequence. The PPR code can then be inferred to be the most recent PPR code predictive model after the iteratively updating is complete. In some implementations, the method further comprises outputting a best matched PPR protein for each editing site.

In some implementations, the method further comprising determining a total best match score after each instance of updating the updated PPR code, wherein a change in the total best match score falling below a predetermined threshold indicates that the best match of a target sequence to the at least one PPR protein does not change any more. In some aspects, the predetermined threshold is less than or equal to 0.0001.

In certain implementations, each PPR codon comprises an amino acid triplet of amino acids at a second position, fifth position, and last position of a PPR motif of the at least one PPR protein. In some aspects, the PPR code comprises a preference of the amino acid triplet of each PPR motif for each nucleotide base.

In some aspects, the background base composition comprises a probability for nucleotide base A of 0.29, a probability for nucleotide base C of 0.15, a probability for nucleotide base G of 0.21, and a probability for nucleotide base U of 0.35. In some aspects, the initial nucleotide base preference for each PPR codon is based on nucleotide probability parameters in the following table:

| PPR-Type | Pos. 5 | Pos. L | A | C | G | U |
|---|---|---|---|---|---|---|
| P or S | T\|S | N | 0.9 | 0 | 0.1 | 0 |
| P or S | T\|S | D | 0.1 | 0 | 0.9 | 0 |
| P or S | T\|S | Not (N\|D) | 0.5 | 0 | 0.5 | 0 |
| P or S | N | N\|S | 0 | 0.6 | 0 | 0.4 |
| P or S | N | D | 0 | 0.3 | 0 | 0.7 |
| | N | Not | 0 | 0.5 | 0 | 0.5 |
| | | (N\|D\|S) | | | | |

| | | | | |
|---|---|---|---|---|
| All others (same as background) | 0.29 | 0.15 | 0.21 | 0.35 |

In other implementations, the method for matching PPR proteins with target sequences in an organism and inferring a PPR code comprises receiving input data related to PPR editing site in the organism and PPR proteins expressed in the organism; estimating a background base composition for a PPR code; assigning an initial nucleotide base preference for each PPR codon of the PPR proteins to determine an initial PPR code predictive model; and calculating an initial scoring matrix for the initial PPR code predictive model. The method further comprises updating the initial PPR code predictive model by performing an iterative expectation-maximization procedure until a best match of a target sequence to a PPR protein does not change any more, indicating a match between the PPR protein and target sequence, wherein the target sequence comprises an editing site; and inferring the PPR code to be the most recent PPR code predictive model after the updating is complete.

In some aspects, the step of estimating the background base composition for the PPR code is based on flanking 46-nucleotide sequences upstream of the editing sites (positions −49 to −4). In certain implementations, the step of assigning the initial nucleotide base preference for each PPR codon of the PPR proteins is based on nucleotide probability parameters. In some aspect, the nucleotide probability parameters are derived from the following table:

| PPR-Type | Pos. 5 | Pos. L | A | C | G | U |
|---|---|---|---|---|---|---|
| P or S | T\|S | N | 0.9 | 0 | 0.1 | 0 |
| P or S | T\|S | D | 0.1 | 0 | 0.9 | 0 |
| P or S | T\|S | Not (N\|D) | 0.5 | 0 | 0.5 | 0 |
| P or S | N | N\|S | 0 | 0.6 | 0 | 0.4 |
| P or S | N | D | 0 | 0.3 | 0 | 0.7 |
| | N | Not | 0 | 0.5 | 0 | 0.5 |
| | | (N\|D\|S) | | | | |

| | | | | |
|---|---|---|---|---|
| All others (same as background) | 0.29 | 0.15 | 0.21 | 0.35 |

A method for predicting whether an editing site is a site for U-to-C editing is also disclosed herein. The method comprises receiving input data related to PPR editing site in the organism and PPR proteins expressed in the organism; estimating a background base composition for a PPR code; assigning an initial nucleotide base preference for each PPR codon of the PPR proteins to determine an initial PPR code predictive model; calculating an initial scoring matrix for the initial PPR code predictive model; and updating the initial PPR code predictive model by performing an iterative expectation-maximization procedure until a best match of a target sequence to a PPR protein does not change any more, indicating a match between the PPR protein and target sequence, wherein the target sequence comprises an editing site. The method next comprises determining the presence of a DYW:JP domain in the PPR protein corresponding to the editing site, wherein the presence of the DYW:JP domain indicates the editing site is a site for U-to-C editing. In some implementations, the step of updating the initial PPR code predictive model comprises using the initial scoring matrix to score each target sequence with respect to the at least one PPR protein; assigning each target sequence to the at least one PPR protein with a probability; estimating a total number of target sequences assigned to the at least one PPR protein; estimating a total number of each nucleotide base assigned to each PPR codon of the at least one PPR protein based on the estimated total number of target sequences assigned to the at least one PPR protein; and then updating the nucleotide probability parameters based on the estimated total number of each nucleotide base assigned to each PPR codon.

In another aspects, a method for predicting whether an editing site is a site for C-to-U editing is disclosed herein. The method comprises receiving input data related to PPR editing site in the organism and PPR proteins expressed in the organism; estimating a background base composition for a PPR code; assigning an initial nucleotide base preference for each PPR codon of the PPR proteins to determine an initial PPR code predictive model; calculating an initial scoring matrix for the initial PPR code predictive model; and updating the initial PPR code predictive model by performing an iterative expectation-maximization procedure until a best match of a target sequence to a PPR protein does not change any more, indicating a match between the PPR protein and target sequence, wherein the target sequence comprises an editing site. The method next comprises determining the absence of a DYW:JP domain in the PPR protein corresponding to the editing site, wherein the absence of the DYW:JP domain indicates the editing site is a site for C-to-U editing. In some implementations, the step of updating the initial PPR code predictive model comprises using the initial scoring matrix to score each target sequence with respect to the at least one PPR protein; assigning each target sequence to the at least one PPR protein with a probability; estimating a total number of target sequences assigned to the at least one PPR protein; estimating a total number of each nucleotide base assigned to each PPR codon of the at least one PPR protein based on the estimated total number of target sequences assigned to the at least one PPR protein; and then updating the nucleotide probability parameters based on the estimated total number of each nucleotide base assigned to each PPR codon.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Implementations will hereinafter be described in conjunction with the appended and/or included DRAWINGS, where like designations denote like elements.

FIGS. 1A-1G show that the PPRDecoder infers the PPR code while accurately matching PLS-type PPRs with target editing sites. FIG. 1A shows the target RNA recognition by the PLS-type PPR proteins. The PPR array aligns with the RNA with one PPR motif binding to one nucleotide, and the last repeat (S2) binding to the −4 position relative to the editing site. FIG. 1B shows the consensus of an illustrating P1-type PPR motif cluster. The three amino acids at the 2nd, 5th, and last (L) positions directly contact RNA are most critical for specific base recognition based on previous biochemical and structural studies. The amino acid triplet is denoted PPR codon in this work. FIG. 1C shows the schematic of the PPRDecoder algorithm. FIG. 1D shows the correlation of PPR binding scores and RNA editing levels. All editing sites were grouped into equal-sized bins according to the predicted binding score for the best matches. The average and standard error of the mean of the editing levels are shown for sites in each bin. The squared correlation is also indicated. FIG. 1E shows the concordance of RNA editing types and types of PPR proteins with respect to the presence and types of DYW domains. Top left: editing sites matched with DYW:KP-containing PPRs are ranked by binding scores. The type of each editing site green for U-to-C and red for C-to-U is indicated in the color bar at the bottom. The enrichment score (cumulative run statistic) is shown at the top. The dashed line indicates the binding score threshold used to predict high-confidence target sites (including 750 sites predicted as targets of DYW:KP-containing PPRs). Top right: the distribution of U-to-C and C-to-U editing sites among high-confidence targets of DYW:KP-containing PPRs and those without an annotated DYW domain. Bottom left and right: similar to top, but for targets predicted using the previous Gerke et al. PPR code (9). The dashed line indicates the binding score threshold that predicts 750 target sites matched with DYW:KP-containing PPRs for a direct comparison with results from PPRDecoder. FIG. 1F shows the distribution of PPRs based on the number of high-confidence target sites. FIG. 1G shows an example target editing site for a PPR protein, a U-to-C editing event in the cox2i381g gene. Note the PPR array recognizes 18 nt, with 17 nt matches based on the inferred PPR code, except for a cytosine juxtaposed to repeat 13.

FIG. 2 shows the PPR code inferred by PPRDecoder. Only codons that are aligned with ≥50 sites are shown. The most informative codons and their usage in each PPR motif type are shown in FIGS. 9-17. The complete lists are provided in Table 2.

FIGS. 3A-3E show the enrichment or depletion of a single amino acid (or amino acid pairs) at each position (or pair of positions) for PPR motifs recognizing each nucleotide base. FIG. 3A shows single amino acids. FIG. 3B-3E show amino acid pairs at all possible pairs of positions.

FIGS. 4A-4C show the P1-type of 35 amino acids (aa) for illustration (see FIGS. 18-25 for other PPR motif types). FIG. 4A shows the two-dimensional projection of PPR motifs using top PCs. Each dot represents one PPR motif. Shading indicates clusters identified in panel FIG. 4B. FIG. 4B shows hierarchical clustering of PPR motifs using the top 10 PCs. Identified clusters are indicated FIG. 4C. FIG. 4C shows consensus amino acids for PPR motifs in each cluster identified in FIG. 4B. Above each logo, the nucleotide preference of repeats of the respective cluster is indicated on the left, and the number of repeats from different types of PPR proteins with respect to the presence and types of DYW domains (see the legend on the bottom right) is shown on the right.

FIGS. 5A-5C show the inference of C-to-U or U-to-C editor types based on the subtypes of PPR motifs. FIG. 5A shows clustering of PPR proteins based on the occurrence of PPR motifs in 42 clusters (subtypes). Each column is a PPR protein, and each row is a PPR motif cluster, and the color in the heatmap represents the number of occurrences. The annotations of DYW domains are shown at the bottom with PPR proteins shown in the same order as in the heatmap. The inferred U-to-C (iU2C) or inferred C-to-U (iC2U) editing factors are indicated. For a select subset of PPR motif clusters that are differentially enriched in different editor types, the consensus amino acid logos are shown on the right. FIG. 5B shows the breakdown of inferred editor types for PPRs containing DYW:PGW domain, DYW:KP domain, and those without a detectable DYW domain. FIG. 5C shows the breakdown of C-to-U or U-to-C editing sites by the types of PPR proteins.

FIGS. 6A-6D show the distribution of repeat lengths for different types of PPR motifs.

FIG. 7 shows the convergence of the expectation-maximization (EM) procedure in PPRDecoder. The total binding site score of the best matches at each iteration is shown.

FIGS. 8A and 8B provide additional examples of PPR-target pairs predicted by PPRDecoder.

FIGS. 9A-9E show the specificity and usage of top codons for P1-type PPR motifs of 35 aa. Related to FIG. 2. FIG. 9A shows the top 30 codons are ranked by information content, FIGS. 9B-9E. The top 5 codons for each nucleotide base are shown.

FIGS. 10A-10E show the specificity and usage of top codons for P1-type PPR motifs of 36 aa. Related to FIG. 2. FIG. 10A shows the top 30 codons ranked by information content. FIGS. 10B-10E show the top 5 codons for each nucleotide base.

FIGS. 11A-11E show the specificity and usage of top codons for P2-type PPR motifs of 35 aa. Related to FIG. 2. FIG. 11A shows the top 30 codons ranked by information content. FIGS. 11B-11E show the top 5 codons for each nucleotide base.

FIGS. 12A-12E show the specificity and usage of top codons for L1-type PPR motifs of 35 aa. Related to FIG. 2. FIG. 12A shows the top 30 codons ranked by information content. FIGS. 12B-12E show the top 5 codons for each nucleotide base.

FIGS. 13A-13E show the specificity and usage of top codons for L1-type PPR motifs of 37 aa. Related to FIG. 2. FIG. 13A shows the top 30 codons ranked by information content. FIGS. 13B-13E show the top 5 codons for each nucleotide base.

FIGS. 14A-14E show the specificity and usage of top codons for L2-type PPR motifs of 36 aa. Related to FIG. 2. FIG. 14A shows the top 30 codons ranked by information content. FIGS. 14B-14E show the top 5 codons for each nucleotide base.

FIGS. 15A-15E show the specificity and usage of top codons for S1-type PPR motifs of 31 aa. Related to FIG. 2. FIG. 15A shows the top 30 codons ranked by information content. FIGS. 15B-15E show the top 5 codons for each nucleotide base.

FIGS. 16A-16E show the specificity and usage of top codons for S2-type PPR motifs of 32 aa. Related to FIG. 2. FIG. 16A shows the top 30 codons ranked by information content. FIGS. 16B-16E show the top 5 codons for each nucleotide base.

FIGS. 17A-17E show the specificity and usage of top codons for SS-type PPR motifs of 31 aa. Related to FIG. 2. FIG. 17A shows the top 30 codons ranked by information content. FIGS. 17B-17E show the top 5 codons for each nucleotide base.

FIGS. 18A-18C show the clustering of P1-type PPR motifs of 36 aa. Related to FIG. 4. FIG. 18A shows the two-dimensional projection of PPR motifs using top PCs. Each dot represents one PPR motif. Colors indicate clusters identified in FIG. 18B. Amino acid combinations at positions 5 and L that confer different binding specificities, as captured in PC3, are indicated. FIG. 18B shows hierarchical clustering of PPR motifs using the top 10 PCs. FIG. 18C shows logos of consensus amino acids for repeats in each cluster identified in FIG. 18C.

FIGS. 19A-19C show the clustering of P2-type PPR motifs of 35 aa. Related to FIG. 4. FIG. 19A shows the two-dimensional projection of PPR motifs using top PCs. Each dot represents one PPR motif. Colors indicate clusters identified in panel FIG. 19B. FIG. 19B shows the hierarchical clustering of PPR motifs using the top 10 PCs. FIG. 19C shows logos of consensus amino acids for repeats in each cluster identified in panel FIG. 19B.

FIGS. 20A-20C show the clustering of L1-type PPR motifs of 35 aa. Related to FIG. 4. FIG. 20A shows the two-dimensional projection of PPR motifs using top PCs. Each dot represents one PPR motif. Colors indicate clusters identified in FIG. 20B. FIG. 20B shows the hierarchical clustering of PPR motifs using the top 10 PCs. FIG. 20C show logos of consensus amino acids for repeats in each cluster identified in FIG. 20B.

FIGS. 21A-21C show the clustering of L1-type PPR motifs of 37 aa. Related to FIG. 4. FIG. 21A shows the two-dimensional projection of PPR motifs using top PCs. Each dot represents one PPR motif. No apparent clusters were observed. FIG. 21B shows hierarchical clustering of PPR motifs using the top 10 PCs. FIG. 21C shows logos of consensus amino acids for all repeats of this type.

FIGS. 22A-22C show the clustering of L2-type PPR motifs of 36 aa. Related to FIG. 4. FIG. 22A shows the two-dimensional projection of PPR motifs using top PCs. Each dot represents one PPR motif. Colors indicate clusters identified in FIG. 22B. FIG. 22B shows the hierarchical clustering of PPR motifs using the top 10 PCs. FIG. 22C shows logos of consensus amino acids for repeats in each cluster identified in FIG. 22B.

FIGS. 23A-23C show the clustering of S1-type PPR motifs of 31aa. Related to FIG. 4. FIG. 23A shows the two-dimensional projection of PPR motifs using top PCs. Each dot represents one PPR motif. Colors indicate clusters identified in FIG. 23B. FIG. 23B shows the hierarchical clustering of PPR motifs using the top 10 PCs. FIG. 23C shows logos of consensus amino acids for repeats in each cluster identified in FIG. 23B.

FIGS. 24A-24C show the clustering of S2-type PPR motifs of 32 aa. Related to FIG. 4. FIG. 24A shows the two-dimensional projection of PPR motifs using top PCs. Each dot represents one PPR motif. Colors indicate clusters identified in FIG. 24B. FIG. 24B shows the hierarchical clustering of PPR motifs using the top 10 PCs. FIG. 24C shows logos of consensus amino acids for repeats in each cluster identified in FIG. 24B.

FIGS. 25A-25C show clustering of SS-type PPR motifs of 31 aa. Related to FIG. 4. FIG. 25A shows the two-dimensional projection of PPR motifs using top PCs. Each dot represents one PPR motif. Colors indicate clusters identified in FIG. 25B. FIG. 25B shows the hierarchical clustering of PPR motifs using the top 10 PCs. FIG. 25C shows logos of consensus amino acids for repeats in each cluster identified in FIG. 25C.

### DETAILED DESCRIPTION

Detailed aspects and applications of the disclosure are described below in the following drawings and detailed description of the technology. Unless specifically noted, it is intended that the words and phrases in the specification and the claims be given their plain, ordinary, and accustomed meaning to those of ordinary skill in the applicable arts.

In the following description, and for the purposes of explanation, numerous specific details are set forth in order to provide a thorough understanding of the various aspects of the disclosure. It will be understood, however, by those skilled in the relevant arts, that embodiments of the technology disclosed herein may be practiced without these specific details. It should be noted that there are many different and alternative configurations, devices, and technologies to which the disclosed technologies may be applied. The full scope of the technology disclosed herein is not limited to the examples that are described below.

The singular forms “a,” “an,” and “the” include plural referents unless the context clearly dictates otherwise. Thus, for example, reference to “a step” includes reference to one or more of such steps.

The word “exemplary,” “example,” or various forms thereof are used herein to mean serving as an example, instance, or illustration. Any aspect or design described herein as “exemplary” or as an “example” is not necessarily to be construed as preferred or advantageous over other aspects or designs. Furthermore, examples are provided solely for purposes of clarity and understanding and are not meant to limit or restrict the disclosed subject matter or relevant portions of this disclosure in any manner. It is to be appreciated that a myriad of additional or alternate examples of varying scope could have been presented but have been omitted for purposes of brevity.

When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable.

Throughout the description and claims of this specification, the words “comprise” and “contain” and variations of the words, for example “comprising” and “comprises”, mean “including but not limited to”, and are not intended to (and do not) exclude other components.

As used herein, the term “PPR codon” refers to the amino acid residues at positions 2, 5, and L of a PPR motif. Each PPR codon has a preferred nucleotide base, which serves as the basis of a PPR code.

As used herein the term “PPR code” refers to the nucleotide-base preference of each PPR codon. From the nucleotide-base preference of each PPR codon within a PPR protein, one can predict the target sequences in an organism that the PPR protein would bind. Accordingly, the editing site of a PPR protein can also be predicted from the PPR code. Where the editing function of the PPR protein in known (for example, U-to-C editing), the PPR code can used to predict where the U-to-C editing would occur on a protein.

The term “plurality”, as used herein, means more than one. When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable.

As required, detailed embodiments of the present disclosure are included herein. It is to be understood that the disclosed embodiments are merely exemplary of the invention that may be embodied in various forms. Therefore, specific structural and functional details disclosed herein are not to be interpreted as limits, but merely as a basis for teaching one skilled in the art to employ the present invention. The specific examples below will enable the disclosure to be better understood. However, they are given merely by way of guidance and do not imply any limitation.

The present disclosure may be understood more readily by reference to the following detailed description taken in connection with the accompanying figures and examples, which form a part of this disclosure. It is to be understood that this disclosure is not limited to the specific materials, devices, methods, applications, conditions, or parameters described and/or shown herein, and that the terminology used herein is for the purpose of describing particular embodiments by way of example only and is not intended to be limiting of the claimed inventions.

Described herein is a computational algorithm (also referred to herein as “PPRDecoder”) that simultaneously match PPR proteins with their targets while inferring a quantitative and predictive PPR code statistically in an unbiased, genome-wide manner, without relying on any known PPR-target pairs.

The feasibility and advantage of inferring the RNA recognition code of PLS-type PPR proteins and predicting their target editing sites on a genome-wide scale, without relying on experimental evidence, is demonstrated in the Examples disclosed herein. The Examples extended the current knowledge by quantifying the specificity of all PPR codons and identifying a number of new codons with high base specificity. The usage of different PPR codons varies dramatically in different types of PPR motifs, which is most likely due to the rapid expansion of the protein family. The specificity of codons can vary in different types of PPR motifs, suggesting the relevance of the protein scaffold that provides sequence and structural context that presents the code amino acids at the protein-RNA interaction interface. Together with the observation that PPR motifs of particular types can form distinct clusters that differ in amino acid sequences throughout the repeats, the results warrant consideration of specific PPR motif scaffolds instead of a “consensus” scaffold in the development of designer PPRs, since a single consensus may not provide the optimal representation of natural repeat scaffolds required to achieve the highest specificity.

The PPR code identified in the Examples focused on the comprehensive lists of PLS-type PPRs and RNA editing sites identified in hornwort Anthoceros agrestis (A. agrestis). The PPR code inferred by PPRDecoder has a dramatically improved accuracy. Detection and integration of all patterns (sometimes subtle) in the disclosed rigorous statistical framework led to accurate prediction of the cognate editing factors for about half of all known organelle editing sites, supported by extended and highly specific protein-RNA interactions consistent with the inferred code. The prediction accuracy was estimated to be 96% for U-to-C editing and 93% for C-to-U editing. This accuracy was estimated based on the nearly perfect match of U-to-C editing sites with PPRs containing the recently characterized DYW:KP domain in the C-terminus, while C2U editing sites are mostly predicted as targets of PPRs without detectable canonical DYW:PG domain or DYW:KP domain. Many of these PPRs presumably contain unannotated variants of DYW:PG domain, as demonstrated in a recent study (9). The analysis provides compelling statistical evidence that DYW:PG domains are responsible for U-to-C editing, which occurs in large numbers in several species including hornworts analyzed in this study. The rapid expansion of this subfamily of PPR proteins is also supported by distinct sequence patterns in their PPR motif arrays. While the possibility of DYW:KP domain catalyzing both U-to-C and C-to-U editing was speculated, the disclosed results suggest that this is unlikely the case, as very few were matched to C-to-U editing sites with the improved algorithm. The analyses also suggest that in nearly all cases, a single PPR with a DYW:KP domain should be responsible for both target recognition and catalysis.

Altogether, PPRDecoder provide a significant step forward to understanding the PPR code to help reveal the molecular function of this extraordinary protein family in plants. In addition, insights from the study may also inform the improvement of designer PPRs for various bioengineering applications.

Thus, PPRDecoder is a method for matching PPR proteins with target sequences in an organism and inferring a PPR code as well as, in some implementations, a method for predicting whether an editing site is a site for U-to-C editing or U-to-C editing. The method is carried out without any experimental evidence of PPR-target sequence pairing. In some aspects, the method assumes that each editing site is regulated by only one PPR protein expressed in the organism and that each PPR protein regulates no editing site or at least one editing site.

The method applies to PLS-type PPR proteins. In other implementations, the method applies to P-type PPR proteins. Thus, in some embodiments, the at least one PPR protein comprises a plurality of a single type of PPR motifs. In other embodiments, the at least one PPR protein comprises a plurality of different types of PPR motifs. For example, the different types of PPR motifs are selected from the group consisting of: P1, P2, L1, L2, S1, S2, and SS.

In some aspects of method for matching PPR proteins with target sequences in an organism and inferring a PPR code, the method comprises receiving input data points related to PPR editing sites in the organism and at least one PPR protein expressed in the organism; estimating a background base composition for a PPR code; and assigning an initial nucleotide base preference for each PPR codon of the at least one PPR protein based on nucleotide probability parameters to determine an initial PPR code predictive model. In some aspects, the background base composition is estimated from flanking 46-nucleotide sequences upstream of the editing sites (positions −49 to −4) of the at least one PPR protein. Each PPR codon comprises an amino acid triplet of amino acids at a second position, fifth position, and last position of a PPR repeat of the at least one PPR protein, and the PPR code comprises a preference of the amino acid triplet of each PPR repeat for each nucleotide base. Though it is possible that amino acid positions in addition to residues 2, 5, and L of each PPR motif, which was studied in the Examples, directly contribute to binding specificity. In some aspects, the background base composition comprises a probability for nucleotide base A of 0.29, a probability for nucleotide base C of 0.15, a probability for nucleotide base G of 0.21, and a probability for nucleotide base U of 0.35. In certain implementations, the initial nucleotide base preference for each PPR codon is based on nucleotide probability parameters in Table 1 as shown in the Examples section. In some aspects, the method is a computational method.

The method next comprises calculating an initial scoring matrix for the initial PPR code predictive model and updating the initial PPR code predictive model. The initial PPR code predictive model is updated by: using the initial scoring matrix to score each target sequence with respect to the at least one PPR protein; assigning each target sequence to the at least one PPR protein with a probability; estimating a total number of target sequences assigned to the at least one PPR protein; and estimating a total number of each nucleotide base assigned to each PPR codon of the at least one PPR protein based on the estimated total number of target sequences assigned to the at least one PPR protein. The step of updating the initial PPR code predictive model further comprises updating the nucleotide probability parameters based on the estimated total number of each nucleotide base assigned to each PPR codon; assigning an updated nucleotide base preference for each PPR codon based on the updated nucleotide probability parameters to determine an updated PPR code predictive model; and calculating an updated scoring matrix for the updated PPR code predictive model. The method for matching PPR proteins with target sequences in an organism and inferring a PPR code next comprises iteratively updating the updated PPR code predictive model until a best match of a target sequence to the at least one PPR protein does not change any more, which indicating a match between the at least one PPR protein and the target sequence, and then inferring the PPR code to be the most recent PPR code predictive model after the iteratively updating is complete. In some implementations, the PPR code is inferred separately the different types of PPR motifs. In certain implementations, the method further comprises outputting a best matched PPR protein for each editing site.

In some aspects, the method for matching PPR proteins with target sequences in an organism and inferring a PPR code comprises receiving input data related to PPR editing site in the organism and PPR proteins expressed in the organism and estimating a background base composition for a PPR code. In some aspects, the background base composition is estimated from flanking 46-nucleotide sequences upstream of the editing sites (positions −49 to −4) of the at least one PPR protein. The method further comprises assigning an initial nucleotide base preference for each PPR codon of the PPR proteins to determine an initial PPR code predictive model; calculating an initial scoring matrix for the initial PPR code predictive model; and updating the initial PPR code predictive model by performing an iterative expectation-maximization procedure until a best match of a target sequence to a PPR protein does not change any more. The lack of change indicates a match between the PPR protein and target sequence, and the target sequence comprises an editing site of the PPR protein. Next, the method comprises inferring the PPR code to be the most recent PPR code predictive model after the updating is complete.

In some implementations, the step of updating the initial PPR code predictive model comprises using the initial scoring matrix to score each target sequence with respect to the at least one PPR protein; assigning each target sequence to the at least one PPR protein with a probability; estimating a total number of target sequences assigned to the at least one PPR protein; and estimating a total number of each nucleotide base assigned to each PPR codon of the at least one PPR protein based on the estimated total number of target sequences assigned to the at least one PPR protein. in some aspects, the step of updating the initial PPR code predictive model further comprises updating the nucleotide probability parameters based on the estimated total number of each nucleotide base assigned to each PPR codon; assigning an updated nucleotide base preference for each PPR codon based on the updated nucleotide probability parameters to determine an updated PPR code predictive model; and calculating an updated scoring matrix for the updated PPR code predictive model.

In some aspects, the method for predicting whether an editing site is a site for U-to-C editing or C to U editing comprises receiving input data related to PPR editing site in the organism and PPR proteins expressed in the organism and estimating a background base composition for a PPR code. In some implementations, the background base composition is estimated from flanking 46-nucleotide sequences upstream of the editing sites (positions −49 to −4) of the at least one PPR protein. The method next comprises assigning an initial nucleotide base preference for each PPR codon of the PPR proteins to determine an initial PPR code predictive model; calculating an initial scoring matrix for the initial PPR code predictive model; and updating the initial PPR code predictive model by performing an iterative expectation-maximization procedure until a best match of a target sequence to a PPR protein does not change any more. Thus, in some aspects, the method is a computational method. When the best match of a target sequence to a PPR protein does not change anymore, a match is found between the PPR protein and target sequence. Thus, the target sequence comprises an editing site. The method then comprises determining the presence or absence of a DYW:JP domain in the PPR protein corresponding to the editing site. The presence of the DYW:JP domain indicates the editing site is a site for U-to-C editing. The absence of the absence of the DYW:JP domain indicates the editing site is a site for C-to-U editing.

In some implementations, the step of updating the initial PPR code predictive model comprises using the initial scoring matrix to score each target sequence with respect to the at least one PPR protein; assigning each target sequence to the at least one PPR protein with a probability; estimating a total number of target sequences assigned to the at least one PPR protein; and estimating a total number of each nucleotide base assigned to each PPR codon of the at least one PPR protein based on the estimated total number of target sequences assigned to the at least one PPR protein. in some aspects, the step of updating the initial PPR code predictive model further comprises updating the nucleotide probability parameters based on the estimated total number of each nucleotide base assigned to each PPR codon; assigning an updated nucleotide base preference for each PPR codon based on the updated nucleotide probability parameters to determine an updated PPR code predictive model; and calculating an updated scoring matrix for the updated PPR code predictive model.

The invention is further described by the following numbered paragraphs:
1. A computational method for matching pentatricopeptide repeat (PPR) proteins with target sequences in an organism and inferring a PPR code, the method comprising:
   receiving input sequence data points related to representing PPR editing sites in the organism and at least one PPR protein expressed in the organism;
   estimating a background base composition for a PPR code from flanking 46-nucleotide sequences upstream of the editing sites (positions −49 to −4);
   assigning an initial nucleotide base preference for each PPR codon of the at least one PPR protein based on nucleotide probability parameters to determine an initial PPR code predictive model;
   calculating an initial scoring matrix for the initial PPR code predictive model;
   updating the initial PPR code predictive model by:
      using the initial scoring matrix to score each target sequence with respect to the at least one PPR protein;
      assigning each target sequence to the at least one PPR protein with a probability;
      estimating a total number of target sequences assigned to the at least one PPR protein;
      estimating a total number of each nucleotide base assigned to each PPR codon of the at least one PPR protein based on the estimated total number of target sequences assigned to the at least one PPR protein;
      updating the nucleotide probability parameters based on the estimated total number of each nucleotide base assigned to each PPR codon;
      assigning an updated nucleotide base preference for each PPR codon based on the updated nucleotide probability parameters to determine an updated PPR code predictive model; and
      calculating an updated scoring matrix for the updated PPR code predictive model;
   iteratively updating the updated PPR code predictive model until a best match of a target sequence to the at least one PPR protein does not change any more, indicating a match between the at least one PPR protein and the target sequence; and
   inferring the PPR code to be the most recent PPR code predictive model after the iteratively updating is complete.
2. The method of claim 1, further comprising determining a total best match score after each instance of updating the updated PPR code, wherein a change in the total best match score falling below a predetermined threshold indicates that the best match of a target sequence to the at least one PPR protein does not change any more.
3. The method of paragraph 2, wherein the predetermined threshold is less than or equal to 0.0001.
4. The method of any one of paragraphs 1-3, wherein the sequence data represents all PPR editing sites in the whole genome of the organism and every PPR protein expressed in the organism.
5. The method of any one of paragraphs 1-4, wherein the at least one PPR protein comprises a PLS-type PPR protein.
6. The method of any one of paragraphs 1-5, wherein each PPR codon comprises an amino acid triplet of amino acids at a second position, fifth position, and last position of a PPR motif of the at least one PPR protein.
7. The method of paragraph 6, wherein the PPR code comprises a preference of the amino acid triplet of each PPR motif for each nucleotide base.
8. The method of any one of paragraphs 1-7, wherein the at least one PPR protein comprises a plurality of a single type of PPR motifs.
9. The method of any one of paragraphs 1-7, wherein the at least one PPR protein comprises a plurality of different types of PPR motifs.
10. The method of claim 9, wherein the types of PPR motifs are selected from the group consisting of: P1, P2, L1, L2, S1, S2, and SS.
11. The method of any one of paragraphs 1-10, further comprising outputting a best matched PPR protein for each editing site.
12. The method of any of one of paragraphs 1-11, wherein the method is carried out without any experimental evidence of PPR-target sequence pairing.
13. The method of any one of paragraphs 1-12, wherein the background base composition comprises a probability for nucleotide base A of 0.29, a probability for nucleotide base C of 0.15, a probability for nucleotide base G of 0.21, and a probability for nucleotide base U of 0.35.
14. The method of any one of paragraphs 1-13, wherein assigning the initial nucleotide base preference for each PPR codon is based on nucleotide probability parameters in Table 1.
15. The method of any one of paragraphs 1-14, wherein the method assumes each editing site is regulated by only one PPR protein expressed in the organism, and wherein the method assumes each PPR protein regulates no or at least one editing site.
16. A computational method for matching pentatricopeptide repeat (PPR) proteins with target sequences in an organism and inferring a PPR code, the method comprising:
   receiving input sequence data related to representing PPR editing site in the organism and PPR proteins expressed in the organism;
   estimating a background base composition for a PPR code;
   assigning an initial nucleotide base preference for each PPR codon of the PPR proteins to determine an initial PPR code predictive model;
   calculating an initial scoring matrix for the initial PPR code predictive model;
   updating the initial PPR code predictive model by performing an iterative expectation-maximization procedure until a best match of a target sequence to a PPR protein does not change any more, indicating a match between the PPR protein and target sequence, wherein the target sequence comprises an editing site; and
   inferring the PPR code to be the most recent PPR code predictive model after the updating is complete.
17. The method of paragraph 16, wherein the sequence data represents all PPR editing sites in the whole genome of the organism and every PPR protein expressed in the organism.
18. The method of paragraph 16 or 17, wherein estimating the background base composition for the PPR code is based on flanking 46-nucleotide sequences upstream of the editing sites (positions −49 to −4).
19. The method of any one of paragraphs 16-18, wherein assigning the initial nucleotide base preference for each PPR codon of the PPR proteins is based on nucleotide probability parameters.
20. The method of paragraph 19, wherein the nucleotide probability parameters are derived from Table 1.
21. A computational method for predicting whether an editing site is a site for U-to-C editing, the method comprising:
   receiving input sequencing data related to representing PPR editing site in the organism and PPR proteins expressed in the organism;
   estimating a background base composition for a PPR code;
   assigning an initial nucleotide base preference for each PPR codon of the PPR proteins to determine an initial PPR code predictive model;
   calculating an initial scoring matrix for the initial PPR code predictive model;
   updating the initial PPR code predictive model by performing an iterative expectation-maximization procedure until a best match of a target sequence to a PPR protein does not change any more, indicating a match between the PPR protein and target sequence, wherein the target sequence comprises an editing site; and
   determining the presence of a DYW:JP domain in the PPR protein corresponding to the editing site, wherein the presence of the DYW:JP domain indicates the editing site is a site for U-to-C editing.
22. The computational method of paragraph 20, wherein the sequence data represents all PPR editing sites in the whole genome of the organism and every PPR protein expressed in the organism.
23. A computational method for predicting whether an editing site is a site for C-to-U editing, the method comprising:
   receiving input sequencing data related to representing PPR editing site in the organism and PPR proteins expressed in the organism;
   estimating a background base composition for a PPR code;
   assigning an initial nucleotide base preference for each PPR codon of the PPR proteins to determine an initial PPR code predictive model;
   calculating an initial scoring matrix for the initial PPR code predictive model;
   updating the initial PPR code predictive model by performing an iterative expectation-maximization procedure until a best match of a target sequence to a PPR protein does not change any more, indicating a match between the PPR protein and target sequence, wherein the target sequence comprises an editing site; and
   determining the absence of a DYW:JP domain in the PPR protein corresponding to the editing site, wherein the absence of the DYW:JP domain indicates the editing site is a site for C-to-U editing.
24. The computational method of paragraph 23, wherein the sequence data represents all PPR editing sites in the whole genome of the organism and every PPR protein expressed in the organism.

Further embodiments are illustrated in the following Examples which are given for illustrative purposes only and are not intended to limit the scope of the invention.

Examples

### I. The PPRDecoder Statistical Framework.

This disclosure focuses on PLS-type PPR proteins because the position of the PPR binding site can be precisely determined with the last PPR motif aligned to position −4 relative to the editing site, although the identity of the cognate PPR protein has yet to be determined (a latent variable). This disclosure focuses on A. agrestis, from which a total of 1748 PLS-type PPR proteins with ≥8 PPR motifs have been predicted, together with 2447 editing sites (1132 C-to-U and 1315 U-to-C sites) in the mitochondria and chloroplast transcriptome (9). These PPRs have 33,867 PPR motifs in total and can be grouped into 6 proteins with a classic DYW:PGW domain, 1057 proteins with the newly characterized DYW:KP domain, and 685 proteins with no DYW domain detected.

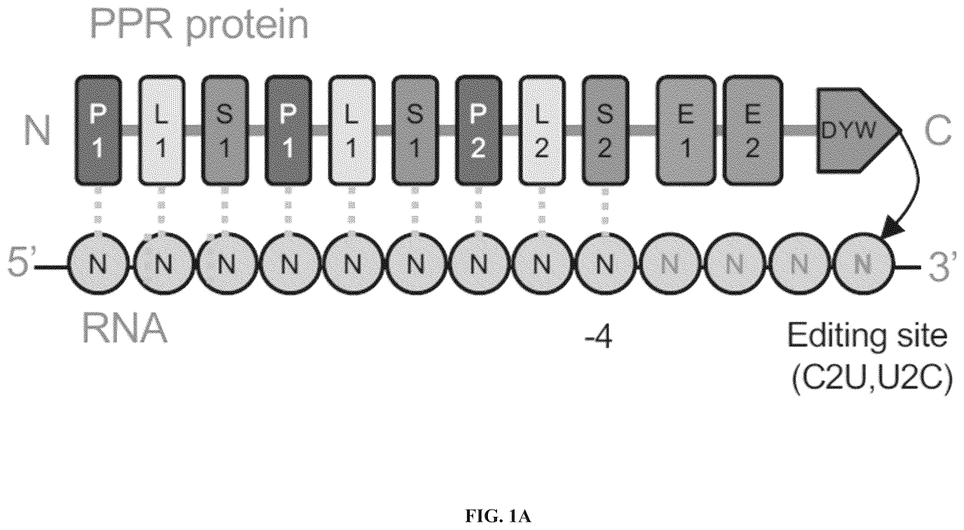

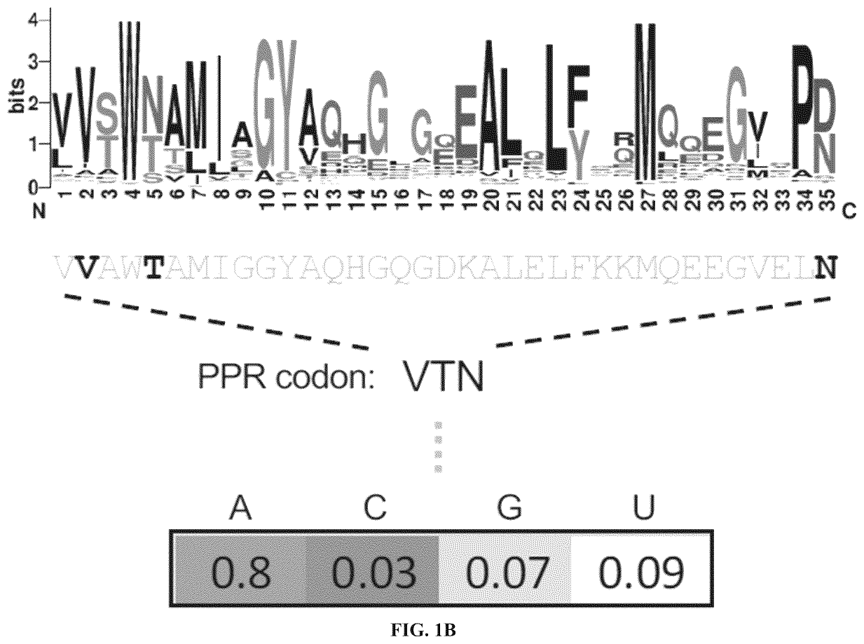

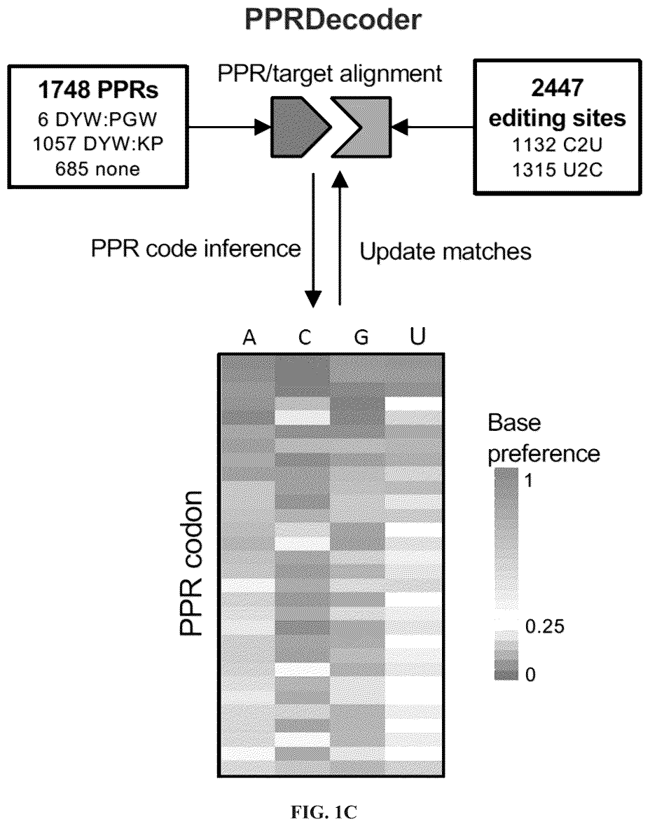

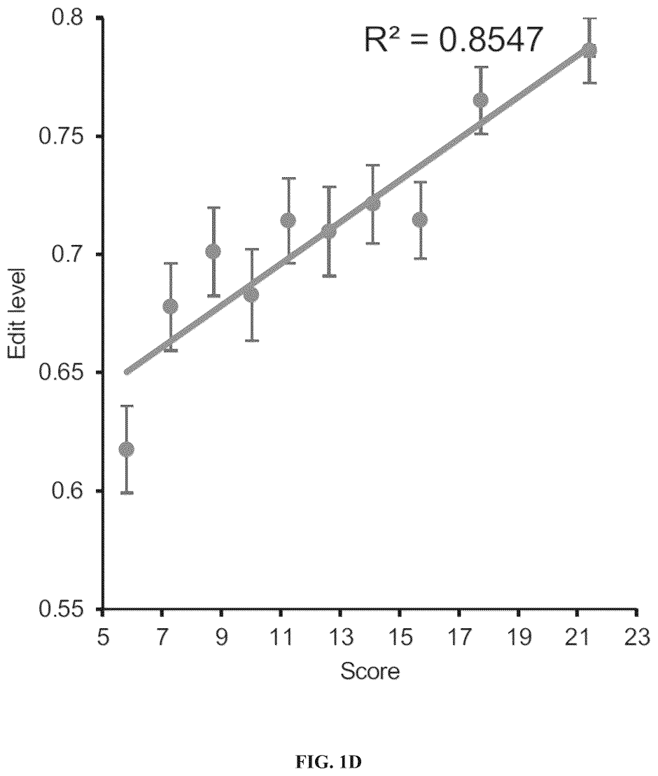

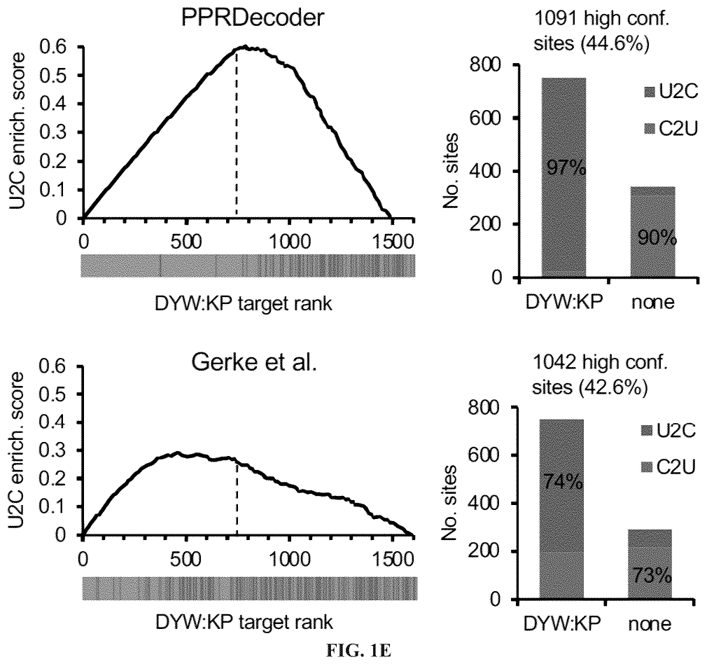

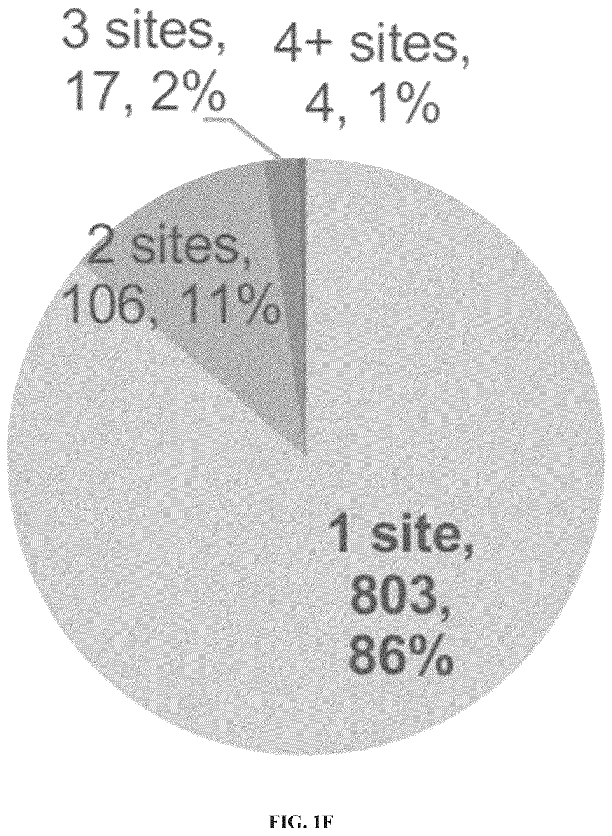

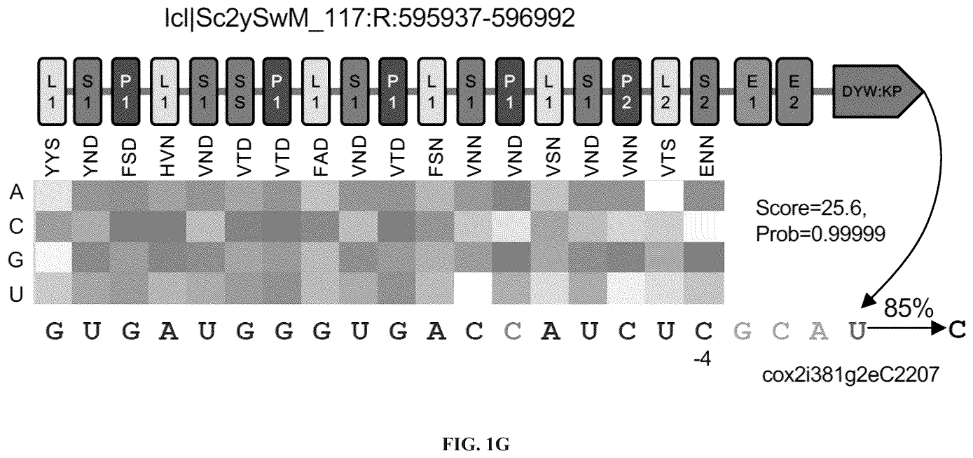

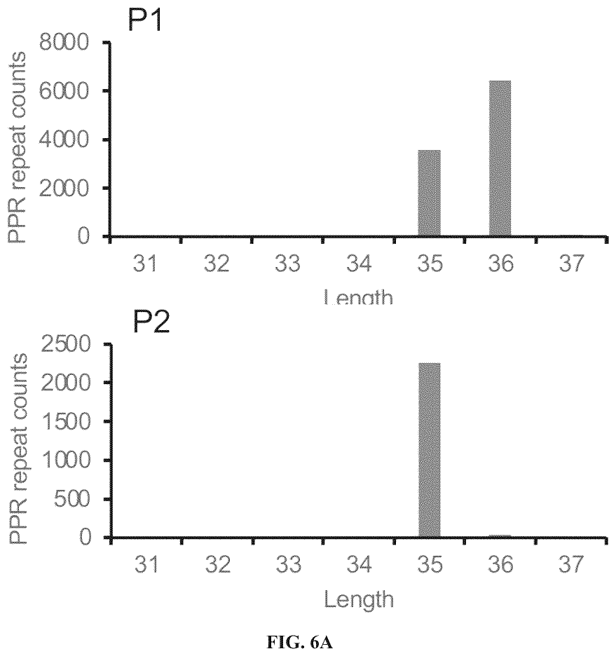

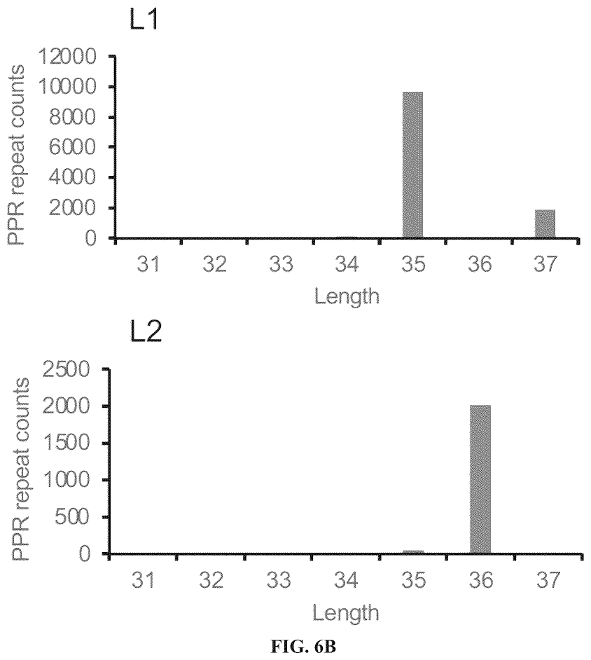

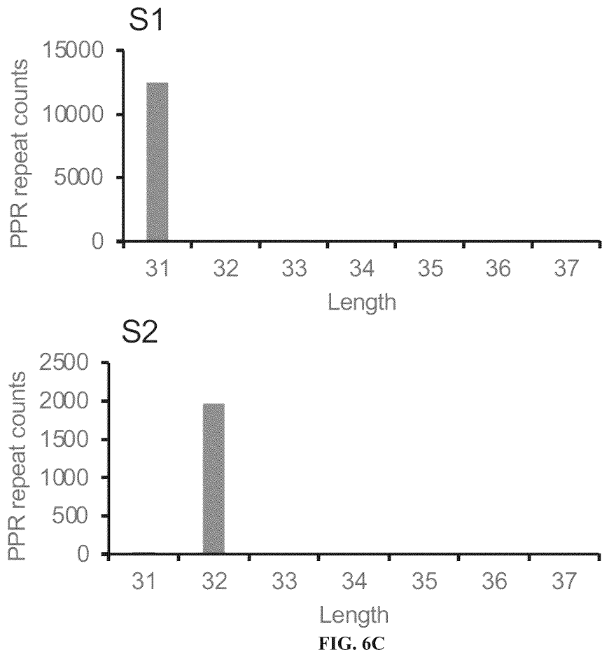

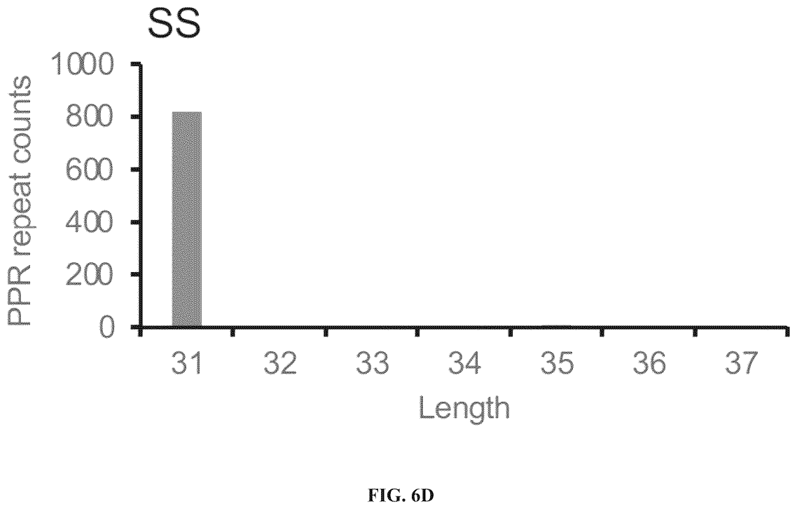

Based on the known PPR-RNA recognition mode, it was assumed that the PPR array precisely registers with the target RNA sequence co-linearly with one-to-one correspondence (FIG. 1A). For each PPR motif, amino acid triplet at positions 2, 5 and L were considered as PPR code amino acids responsible for its target specificity, and each triplet denotes a “PPR codon” (FIG. 1B). With these realistic simplifications, the known specificity of PPR proteins, and the much limited search space by focusing on PLS PPRs and their target editing sites, it was reasoned that the latent PPR-target matches and binding specificity of each PPR protein can be inferred by optimizing the PPR code (the nucleotide-base preference of each PPR codon; model parameters) that maximizes the likelihood of observing the list of PPR binding-sequences flanking the editing sites (the data) using an iterative expectation-maximization procedure (28) (FIG. 1C; Methods). Given the different types of PPR motifs (P1, P2, L1, L2, S1, S2, and SS) which might have different specificity (e.g., the L-type PPR motifs are considered to be less-specific (18)) and that variation in repeat length even for the same repeat type may alter its specificity (FIGS. 6A-6D), it was decided to infer the code separately for each repeat type/length in PPRDecoder without assuming which repeat type might have more contribution to protein-RNA interaction specificity.

### II. The Prediction of C-to-U and U-to-C Editing Sites.

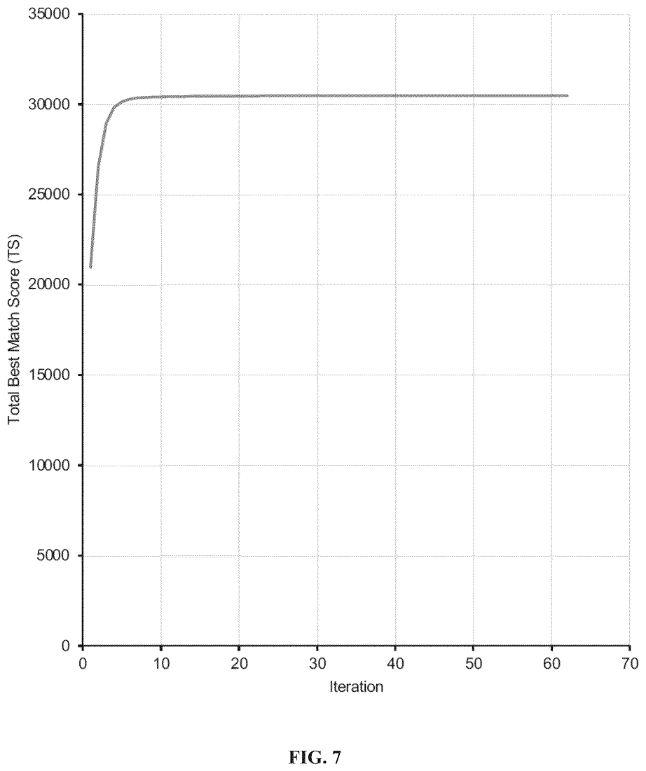

When applied to the A. agrestis data described above, PPRDecoder iteratively improved the quality of alignments between PPR proteins and the best matched target sites, as measured by the binding scores using a position specific weight matrix, a standard scoring method used to evaluate the specificity and binding affinity protein-nucleic acid interactions (29, 30) (FIG. 7). The EM procedure successfully converged and reported the best matched PPR for each editing site with a binding score and posterior probability of the match.

The accuracy of target prediction was evaluated using several metrics. First of all, the U-to-C or C-to-U editing level has been determined for each site from RNA-seq, although the information was not used by PPRDecoder. It was argued that stable protein-RNA complexes should facilitate RNA editing. Indeed, a strong correlation was observed between the predicted binding scores and their editing levels across all editing sites (FIG. 1D).

As a second metric, the concordance between the types of RNA editing was examined and the presence as well as the types of DYW domains suggested that the canonical DYW:PGW domain and several variants with the “PG” box catalyze C-to-U editing, while the newly characterized DYW:KP domain represents a candidate that catalyzes U-to-C editing, based on the expansion of this PPR subfamily correlated with the increased number of U-to-C editing sites (8). Differential enrichment of U-to-C editing sites as DYW:KP targets and C-to-U editing as DYW:PG targets was also observed, although the two populations overlap quite substantially (9). DYW domain annotations were not used in PPRDecoder. However, when PPRs with annotated DYW:KP domains were focused upon, and the ranked list of predicted targets by PPRDecoder was examined, it was noticed that the top predictions with the highest binding scores are nearly exclusively U-to-C editing sites (FIG. 1E, top left panel). Although the total numbers of C-to-U and U-to-C editing sites are relatively similar in A. agrestis organellar transcriptome (46% and 56%, respectively), the highest scoring C-to-U editing site matched to DYW:KP PPRs ranked 111. This nearly exclusive representation of U-to-C editing sites continues among the top 750 DYW:KP targets, corresponding to a binding score of 12.7, a threshold that was chosen to define high-confidence targets. Using this threshold, PPRDecoder additionally predicted 341 targets matched to PPRs without an annotated DYW domain, resulting in a total of 1091 high-confidence target sites, which represent 48.6% of all editing sites. Importantly, among the 750 high-confidence DYW:KP targets, 97% are U-to-C editing sites, whereas among the 341 targets of PPRs without an annotated DYW domain, 90% are C-to-U editing sites (FIG. 1E, top right panel). Among the PPRs without annotated DYW domains used to generate the list of predicted PPR proteins, a subset might actually have variants of DYW:PG domains reported in a recent study (9), which escaped detection by PPRFinder (8). On the other hand, some C-to-U editors are known to lack a DYW domain, and RNA editing is catalyzed by recruiting a second PPR with a DYW domain (31-33). Whether U-to-C editing can also involve such multi-PPR complexes is unknown, although it is noted that among the 762 high-confidence U-to-C editing sites, 750 (98.4%) have DYW:KP domains detected. Nevertheless, the assignments of C-to-U and U-to-C editing sites to PPRs associated with distinct DYW domains with minimal overlap provide compelling support for the accuracy of PPR target prediction by PPRDecoder.

The performance of PPRDecoder was compared to the previous code used for target prediction (Gerke et al. (9)) based on their ability to distinguish C-to-U and U-to-C editing sites. When the list of DYW:KP targets were ranked based on the predicted binding scores using the Gerke et al. code, the U-to-C and C-to-U sites are much more intermingled (FIG. 1E, bottom left panel), as observed in the original study (9). To make a direct comparison with the present results by PPRDecoder, the binding score threshold (≥8.8) was determined so that also the top 750 editing sites matched with DYW:KP-containing PPRs would be predicted. Among this list, only 74% are U-to-C editing sites, which is substantially lower than the fraction among top targets predicted by PPRDecoder (97%; FIG. 1E, bottom right panel). Similarly, among the 292 additional targets matched to other PPRs using the same binding score threshold, 73% are C-to-U editing sites, as compared to 90% by PPRDecoder. Therefore, statistical modeling of all known editing sites and PPR proteins by PPRDecoder substantially improved the accuracy compared to the previous method that uses arbitrary weights that represent base preference of different types of PPR motifs.

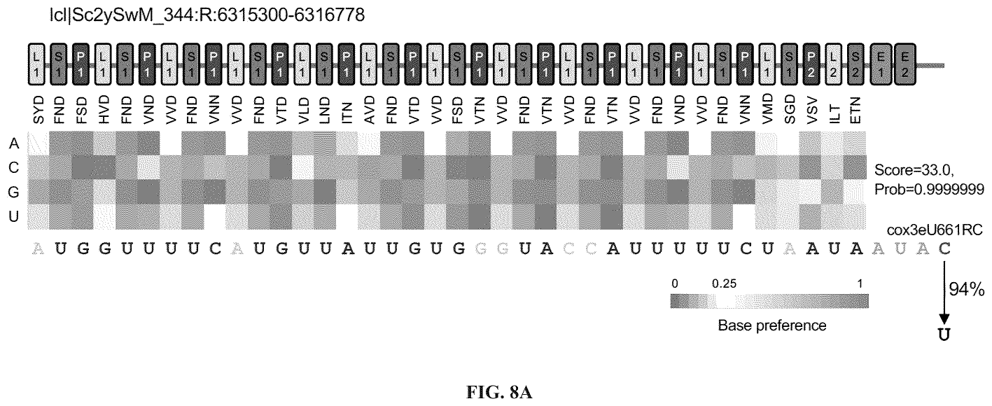

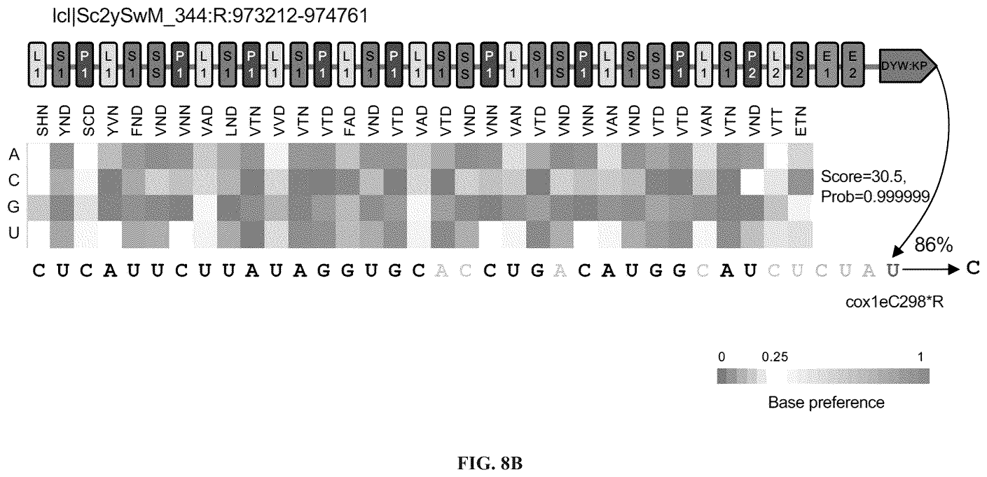

Overall, the 1091 high-confidence target editing sites predicted by PPRDecoder were matched to 930 PPRs, with a vast majority of PPRs have one or two targets (86% and 11%, respectively; FIG. 1F). The very top editing site ranked by the binding score has 38 PPR motifs interacting with RNA co-linearly with 32.9 bits of information, indicating approximately one site per 0.8×10⁹ nucleotides (FIG. 8A). A total of 164 sites have ≥20 bits of information, indicating approximately one site per million nucleotides (FIGS. 1G and 8A-8B), confirming the striking specificity of PPRs.

### III. The Quantitative PPR Code.

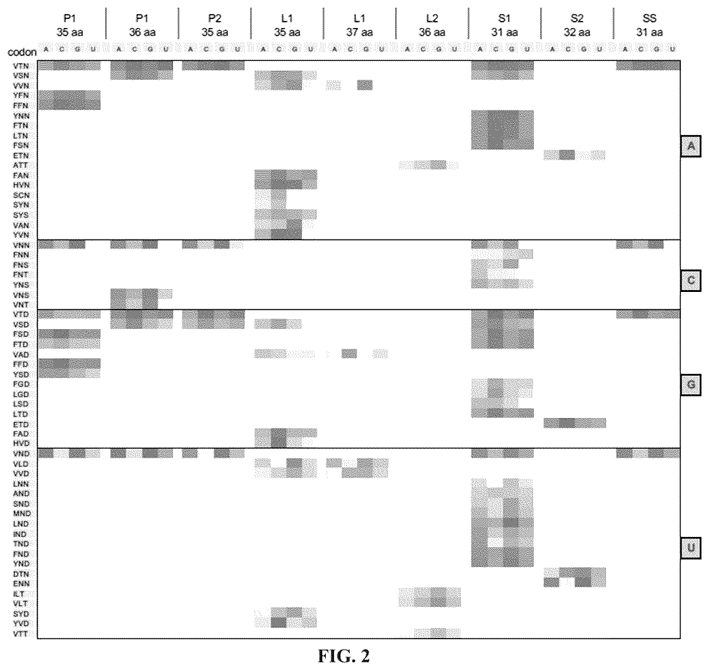

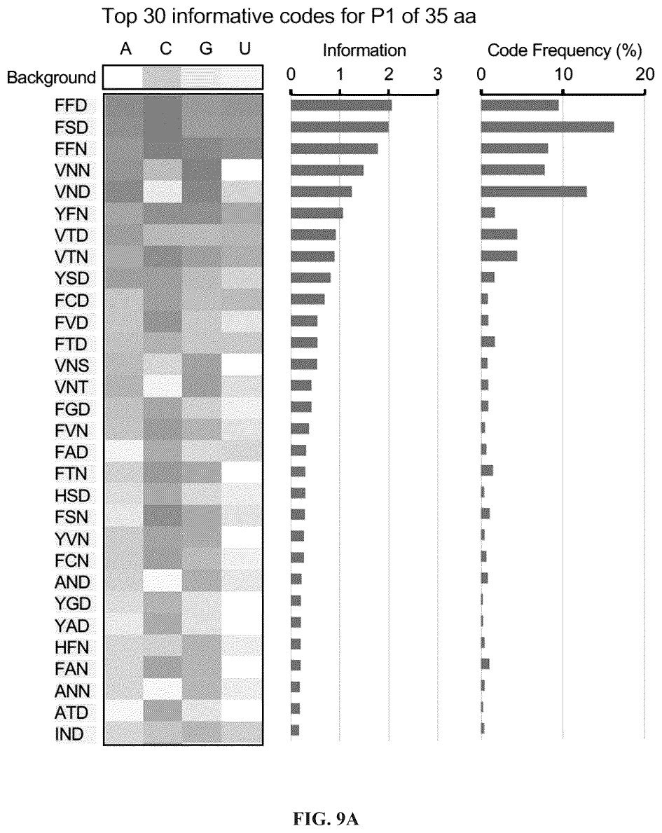

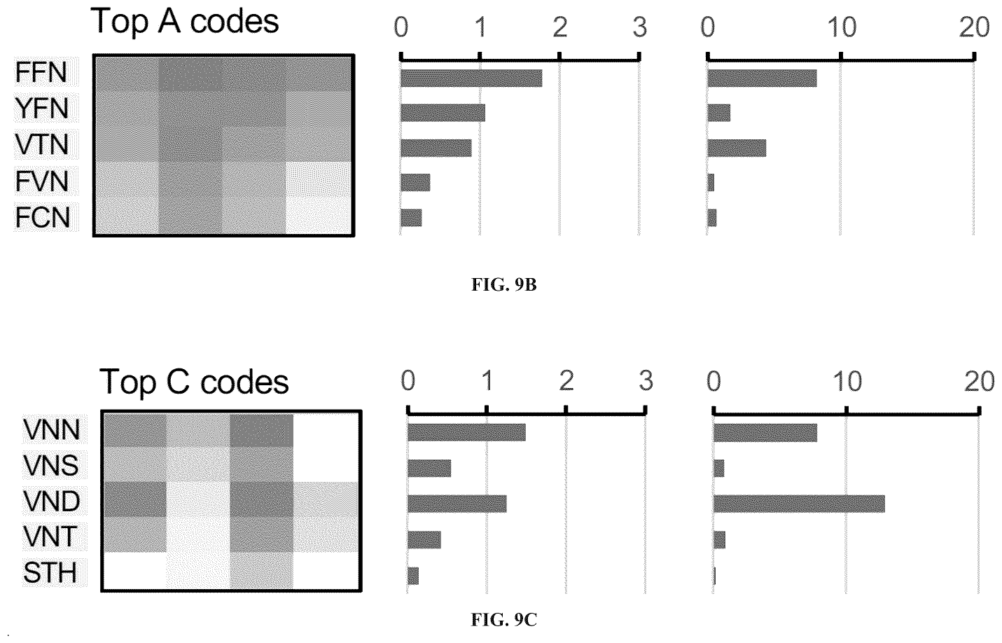

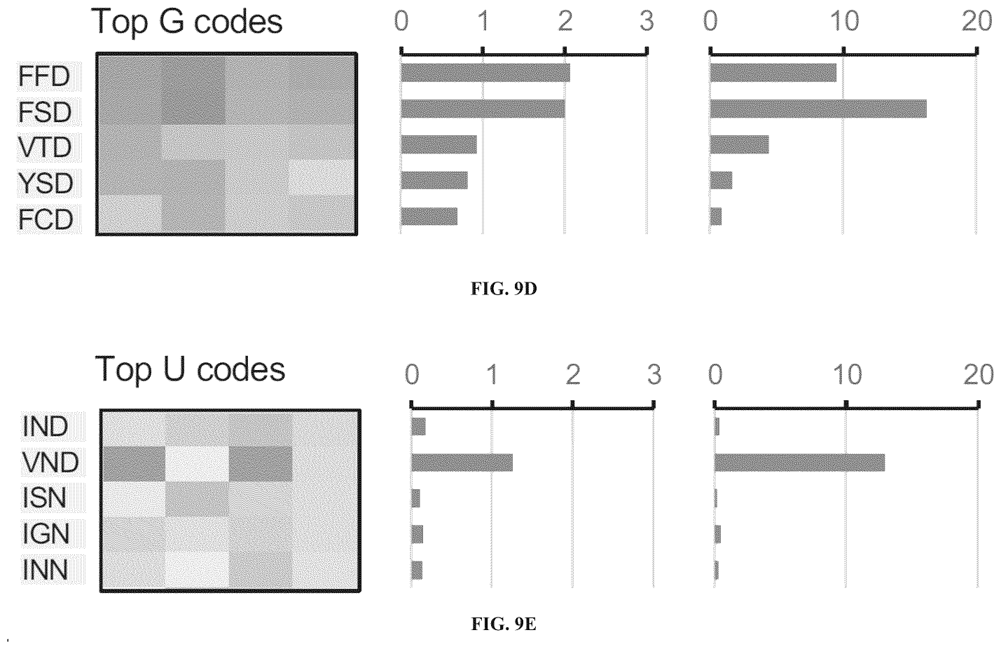

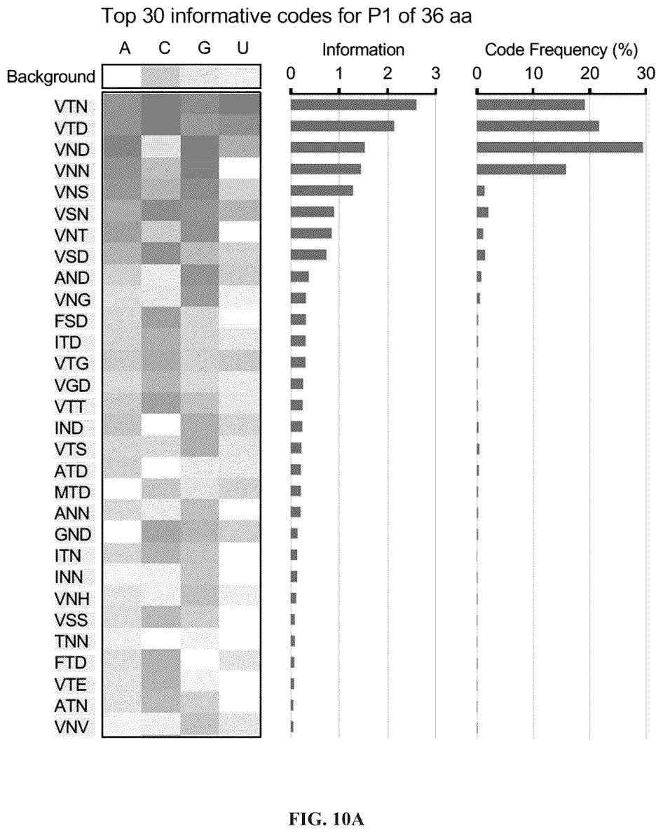

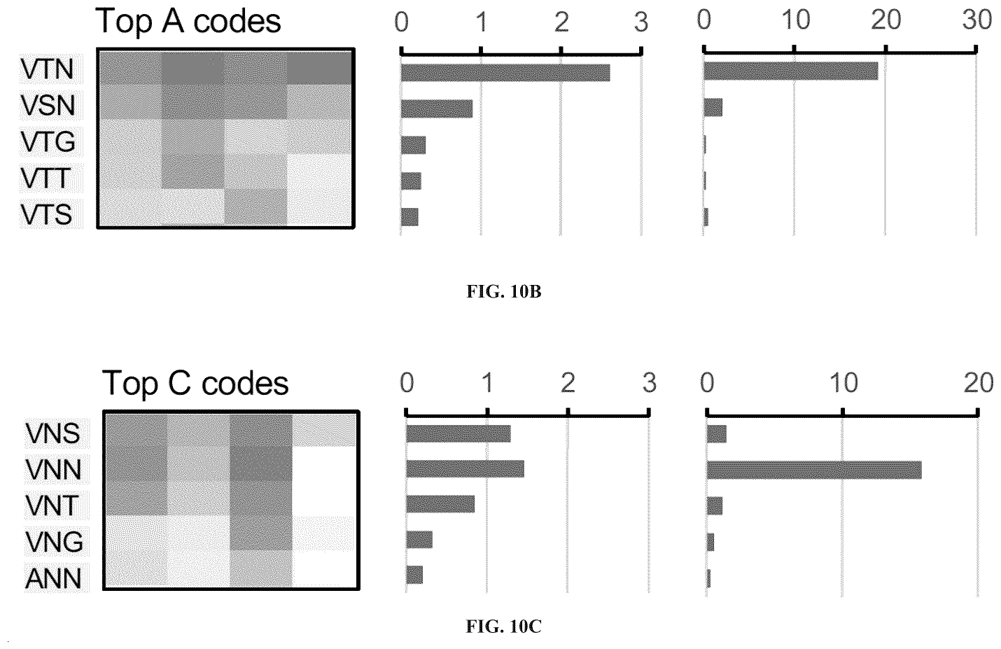

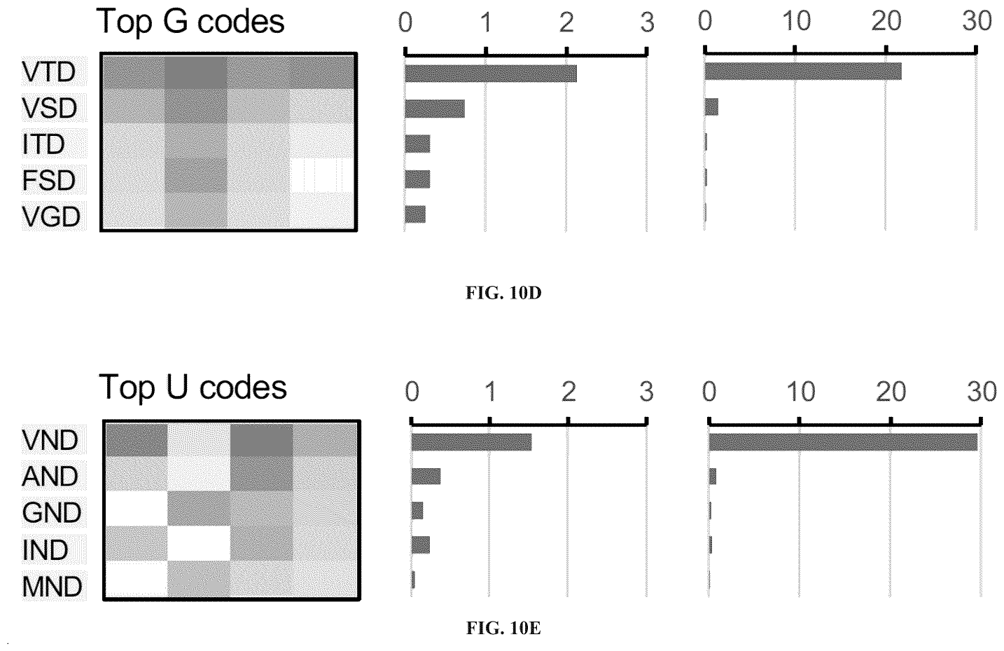

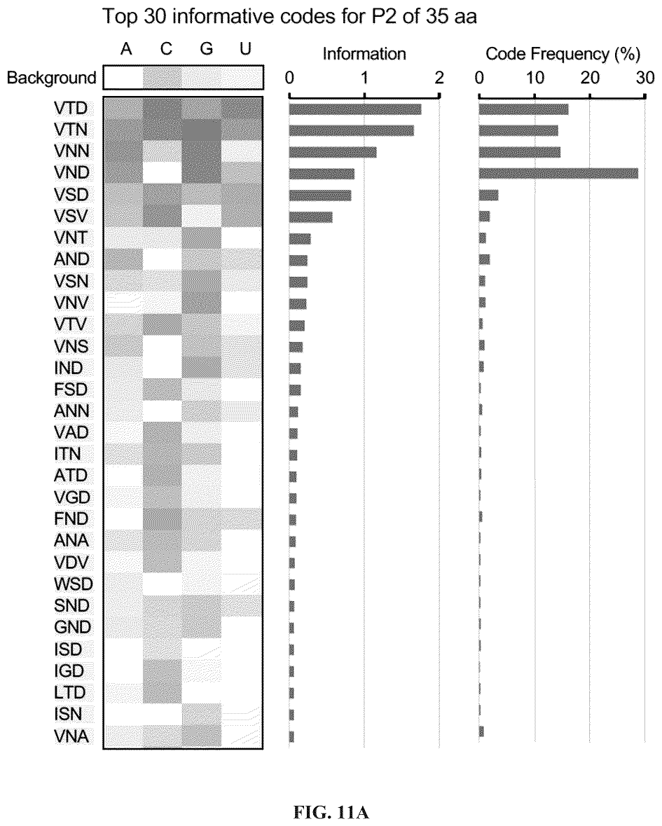

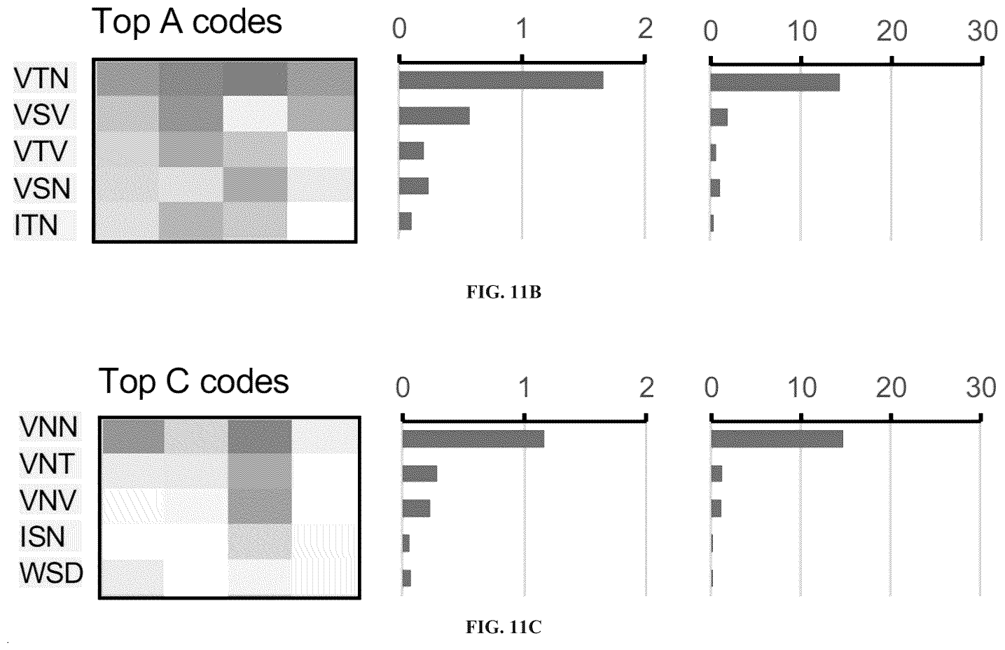

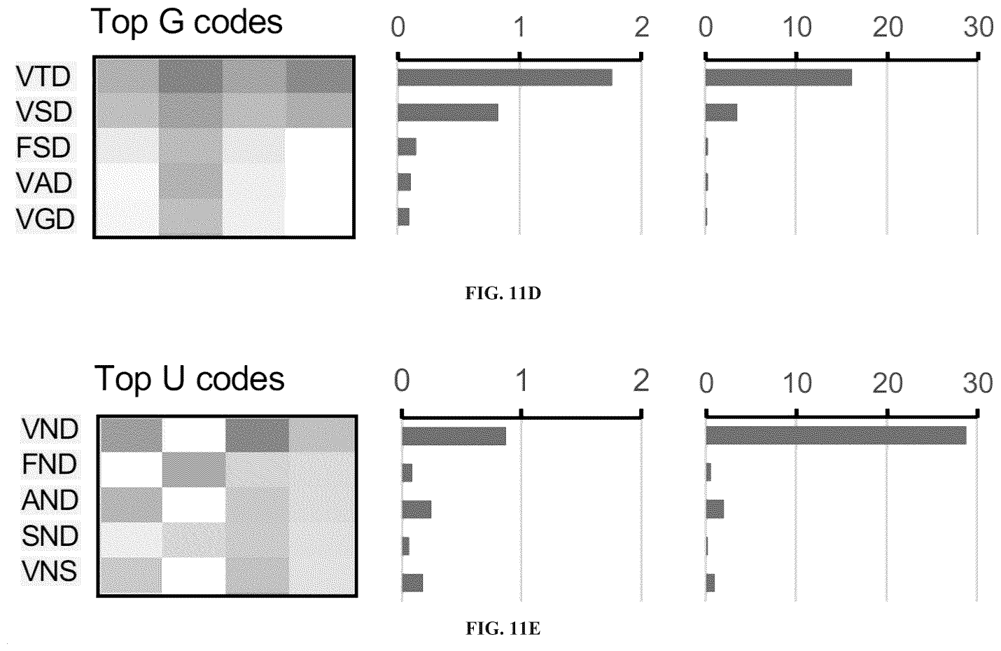

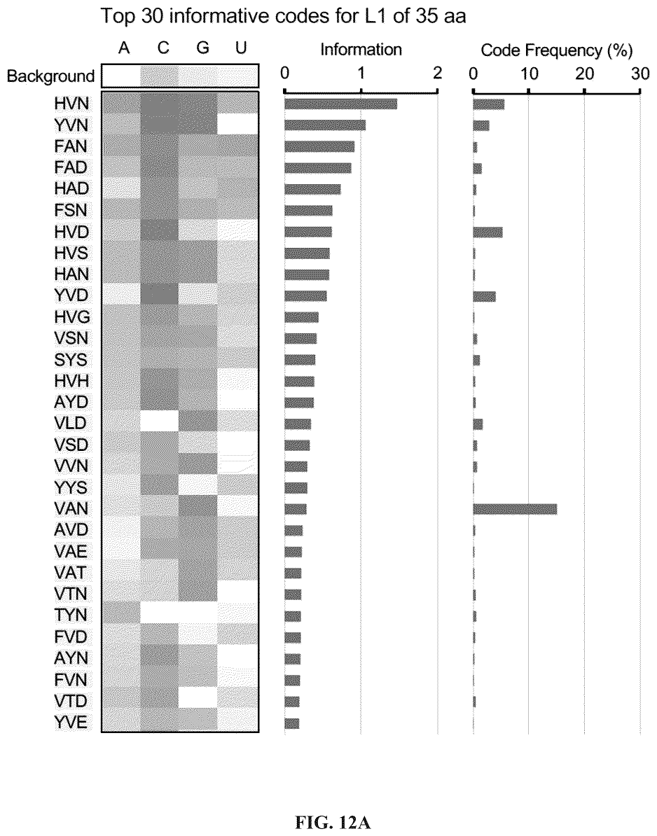

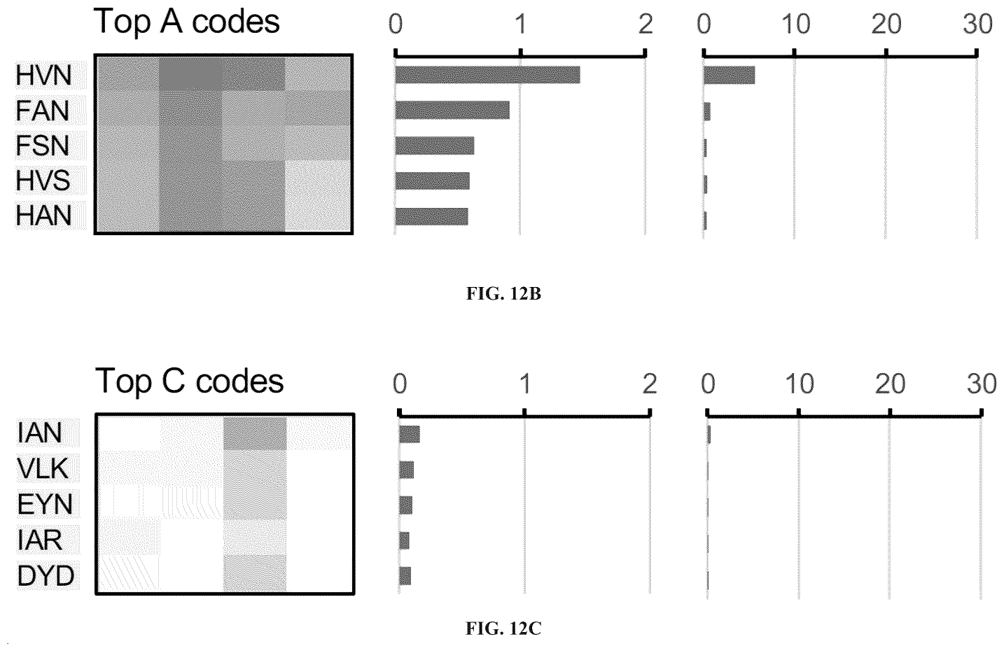

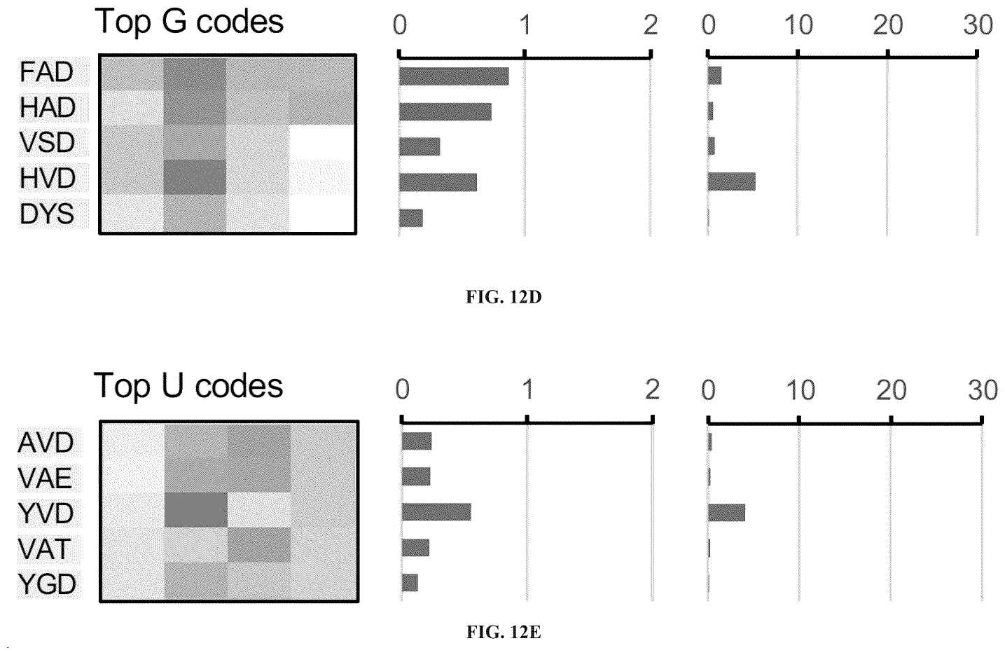

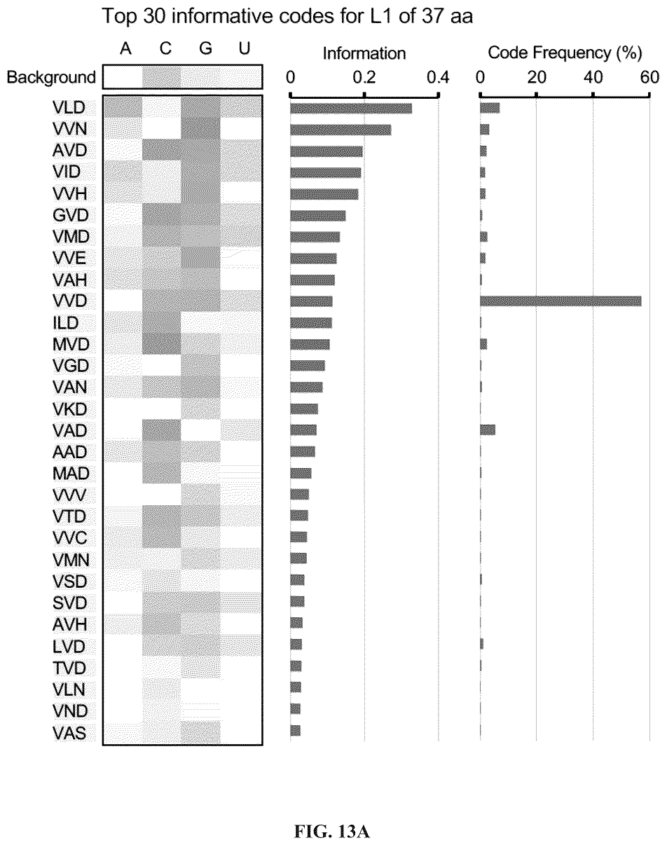

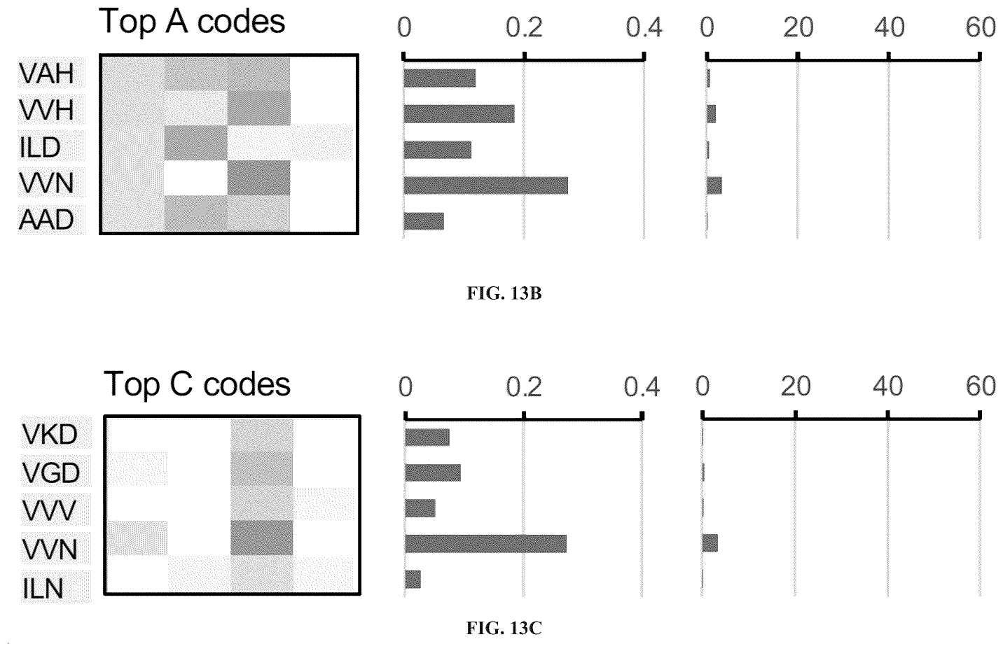

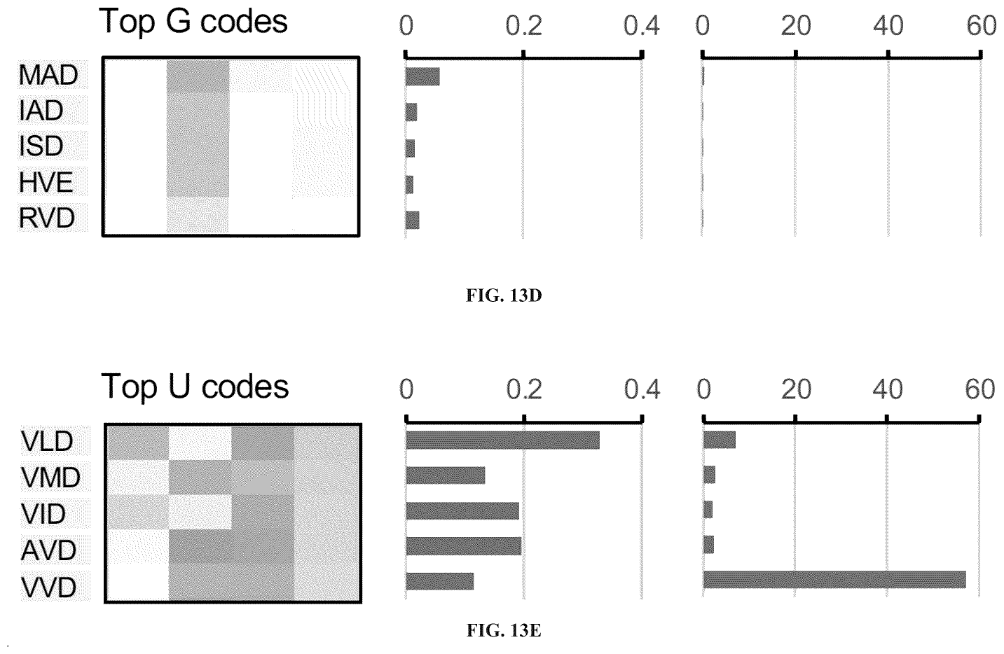

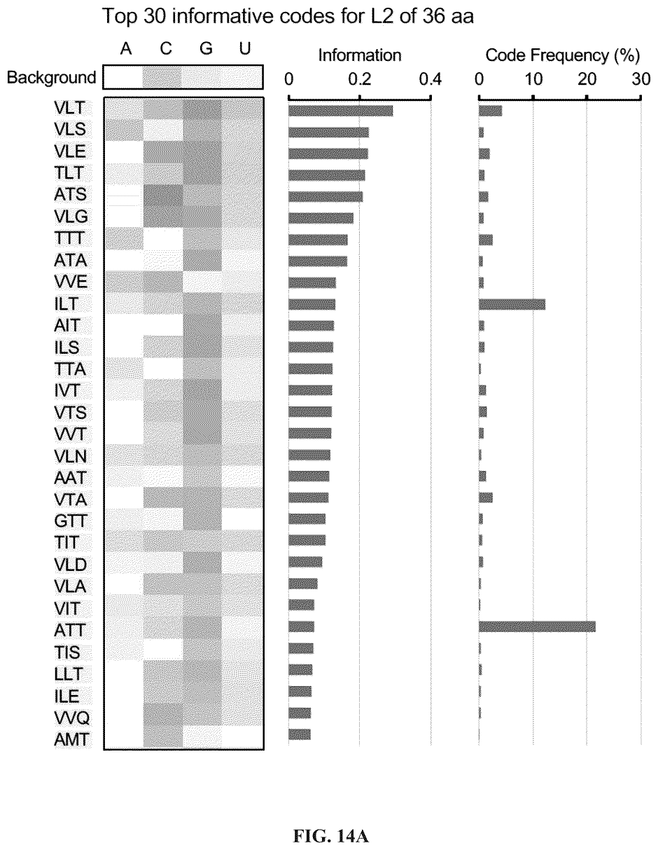

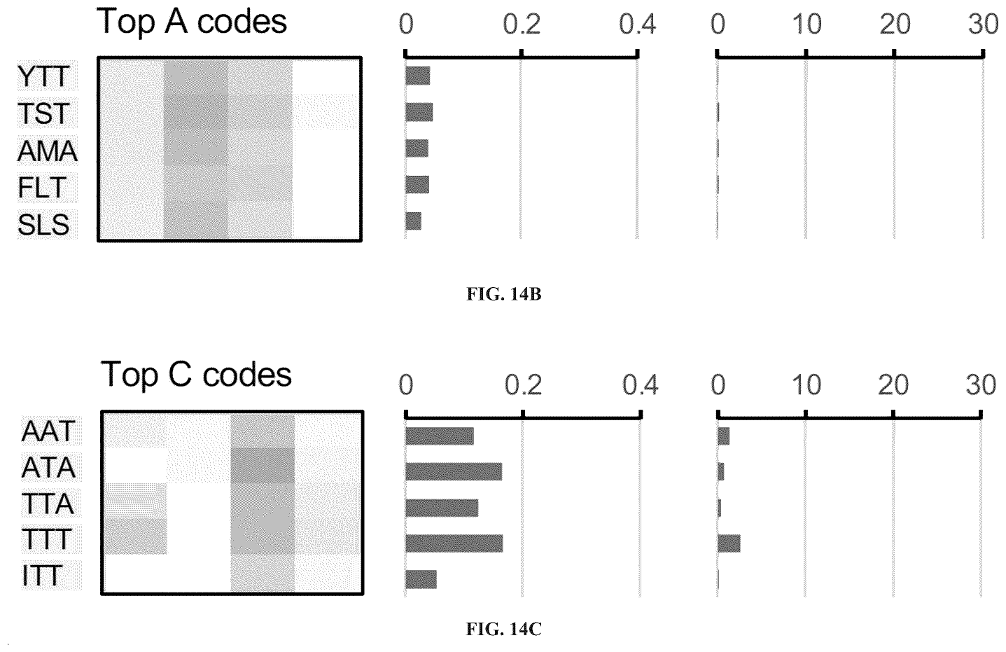

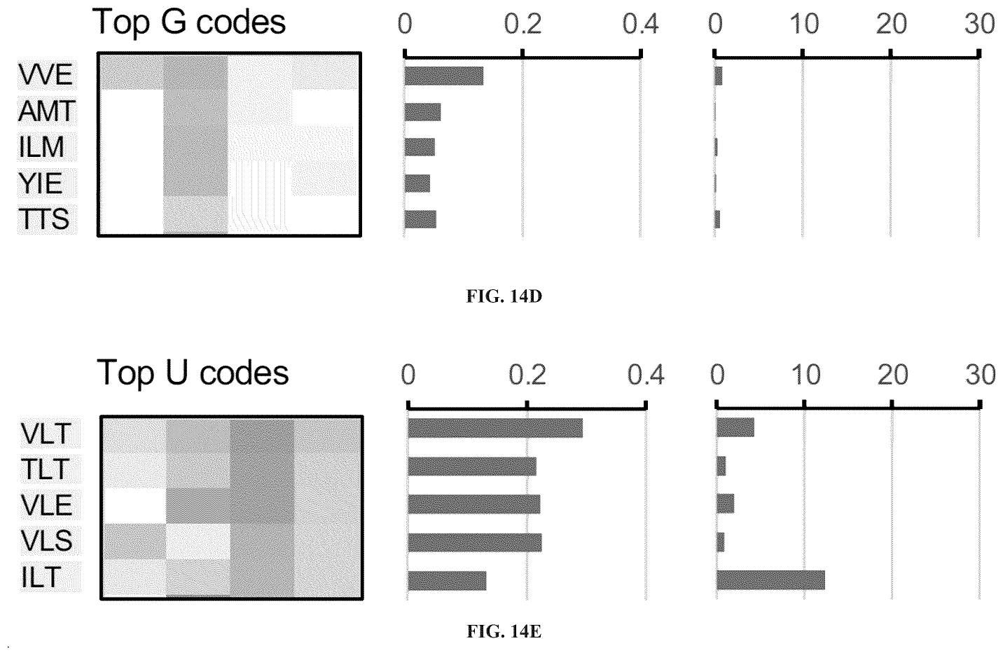

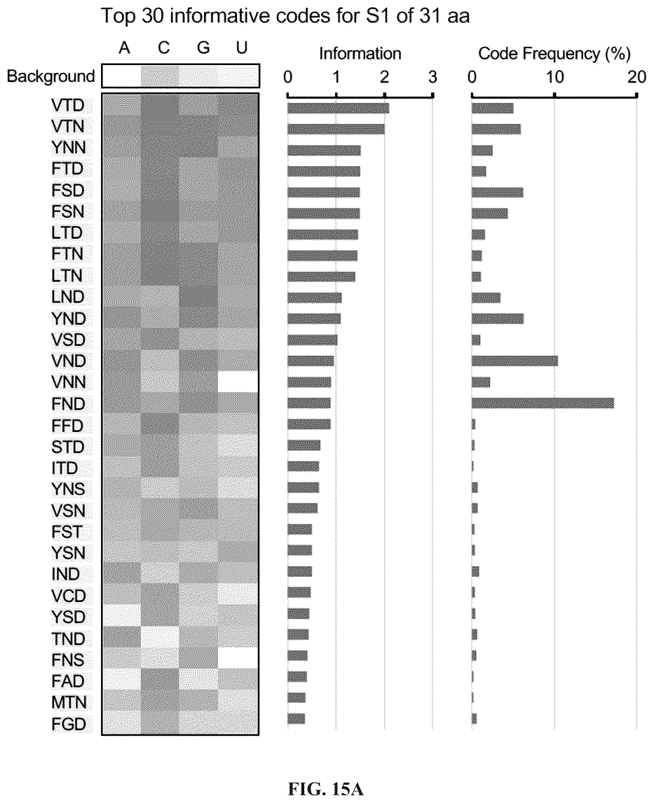

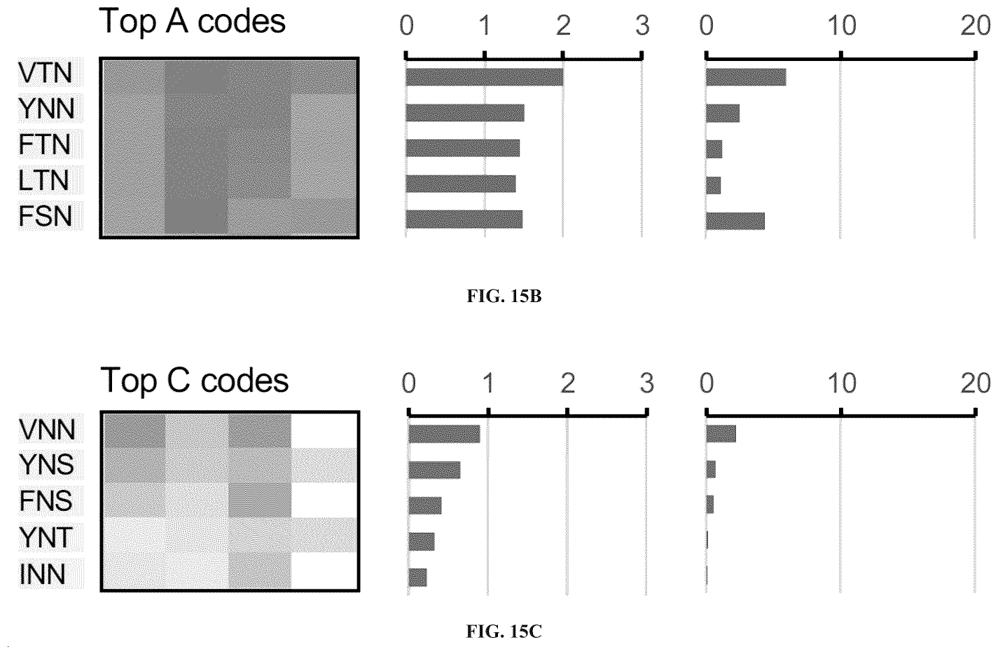

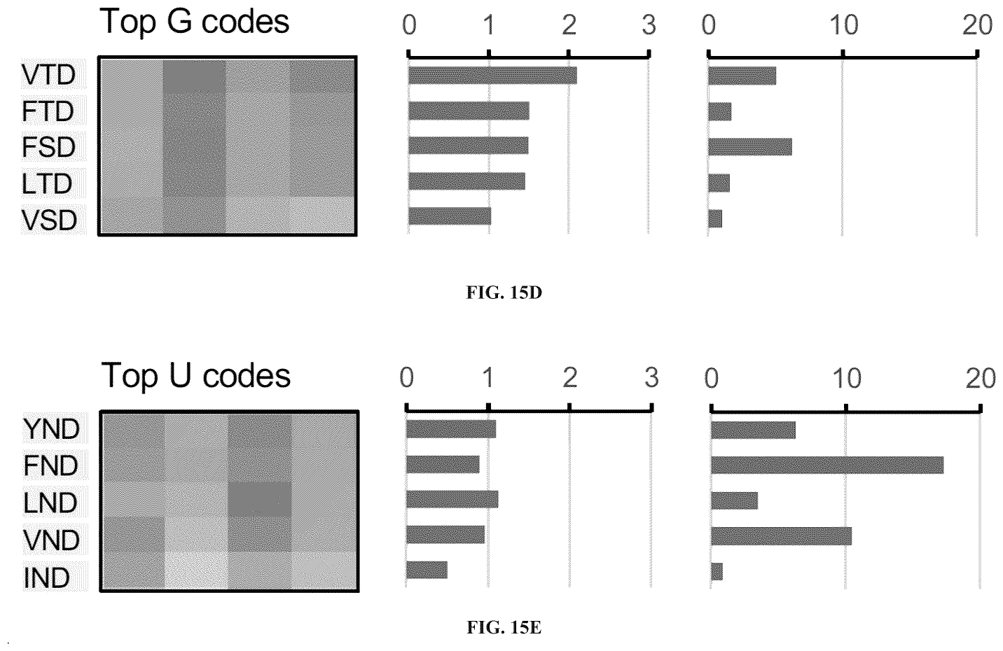

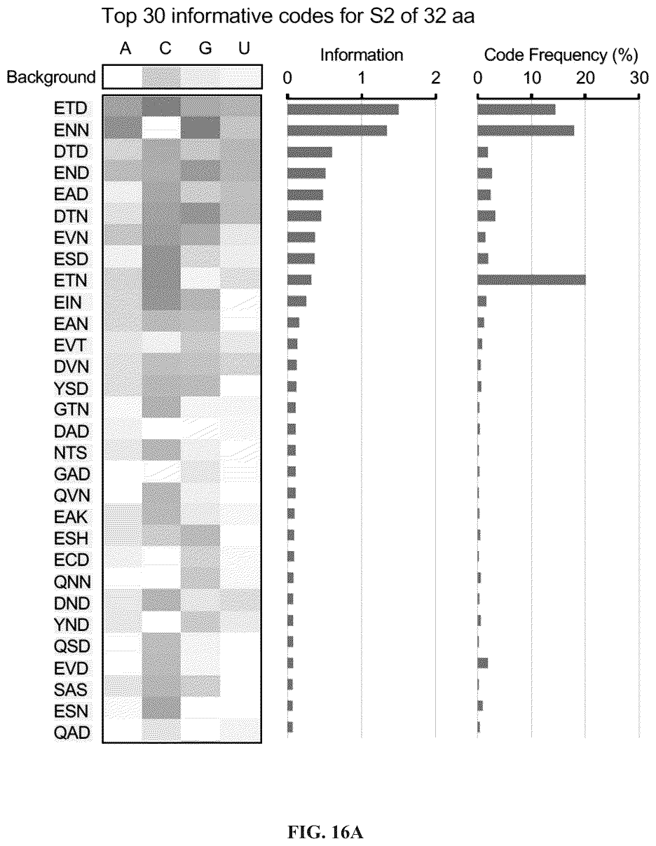

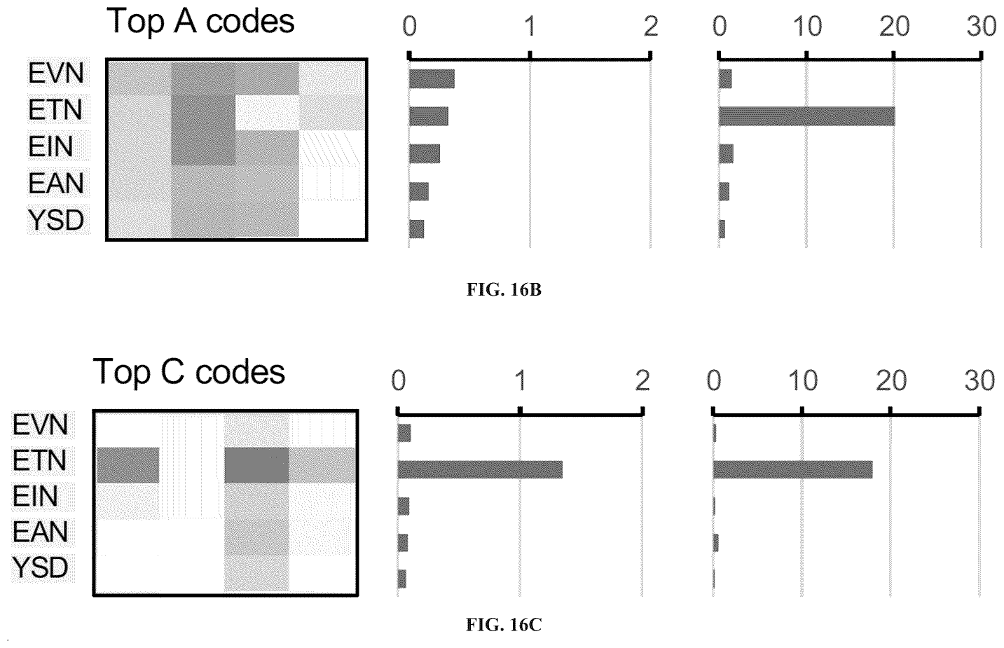

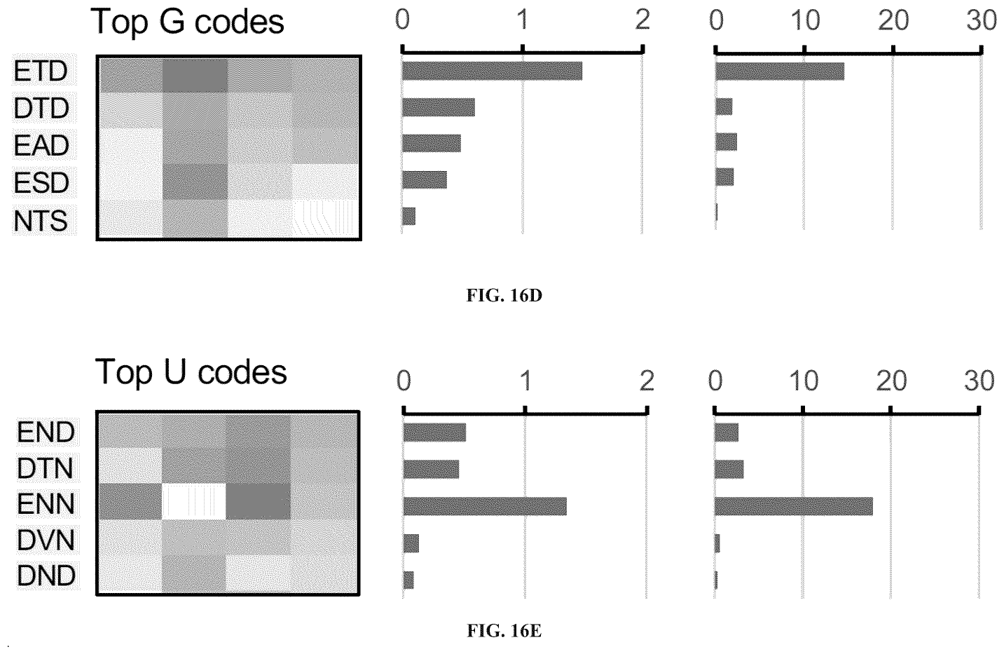

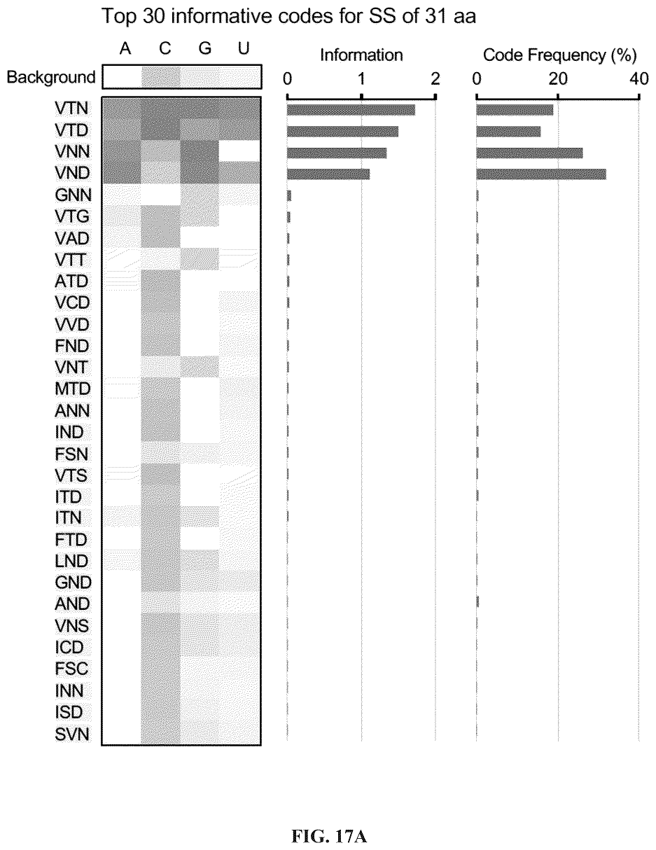

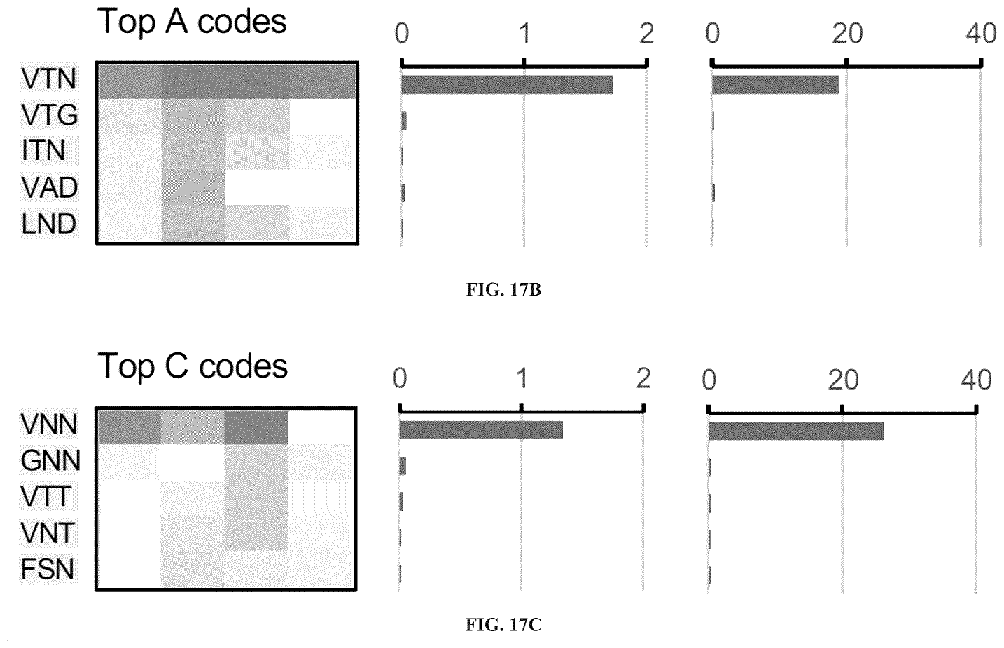

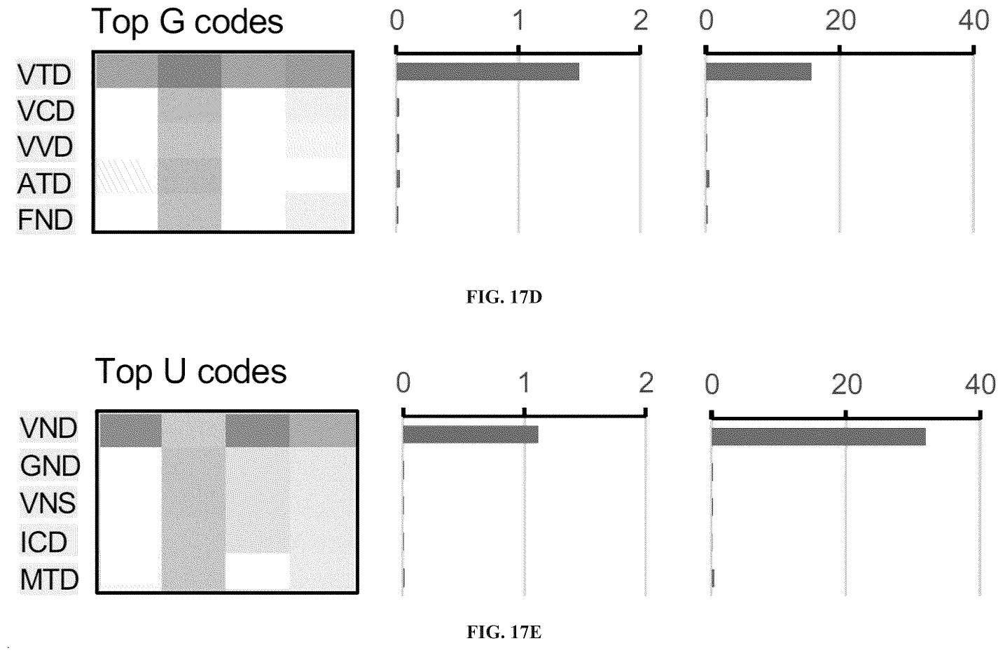

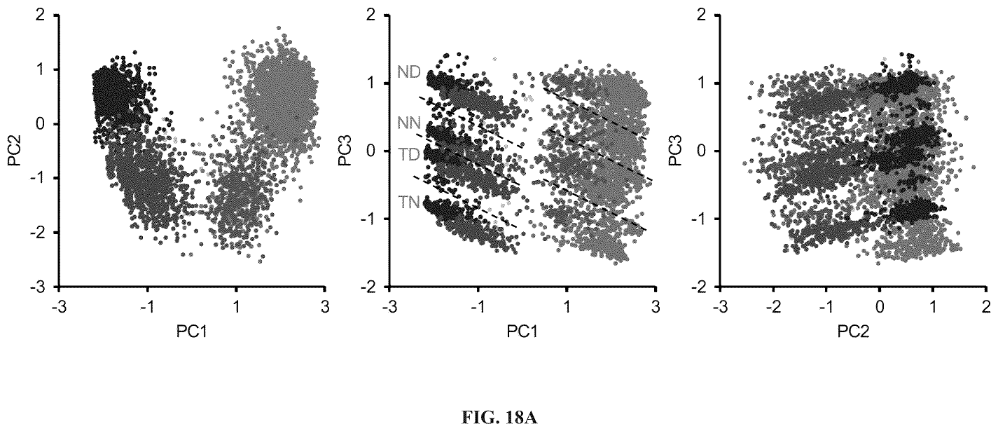

Due to the accuracy of target editing site prediction, the PPR code inferred by PPRDecoder was examined next (Tables 2). Amino acid combinations at positions 2 and L, TN/SN, NN, TD, ND were previously known to have a high preference for A, C, G, and U, respectively. In addition, L-type PPR motifs in general have lower specificity (18). These observations were in general confirmed in the code inferred by PPRDecoder (FIG. 2 and FIGS. 9-17). A more careful examination of the new code revealed several insights when the base specificity was examined as well as usage of PPR codons.

First, in addition to the canonical amino acid combinations characterized in previous studies (18-20), PPRDecoder identified a list of new codons showing high base specificity. In total, PPRDecoder identified 58 codons used by ≥50 sites for at least one repeat type (FIG. 2). Examples of previously uncharacterized codons include those containing phenylalanine at position 5, with YFN/FFN highly specific for A and FFD for G, respectively. In general, more codons show high specificity for A, G, and U, while fewer codons specifically recognize C.

Second, the frequency of PPR codon usage defers dramatically across different types of PPR motifs, or even in the PPR motifs of the same types with different lengths. Among them, it was found that top codons for P1-type PPRs of 35 amino acids (aa) frequently have phenylalanine at positions 2 and 5 (e.g., FFD, YFN, and FFN), while phenylalanine rarely occurs in P1-type PPRs of 36 aa (FIGS. 2, 9A-9E, and 10A-10E). Similarly, L1- and S1-type PPRs each have a number of codons rarely used in other repeat types (FIGS. 2, 12A-12E, and 15A-15E).

Third, while L1-type PPRs are in general less base-specific, PPRDecoder nevertheless identified a number of codons showing relatively high specificity, especially in the L1-type of 35 aa (FIGS. 2 and 12-14). For example, HVN and FAN have a high preference for A (80% and 86%, respectively), while FAD and VLT have a high preference for G (72%) and U (63%), respectively.

Fourth, while the amino acids at positions 5 and L are in general the most critical for binding specificity, the amino acid at position 2 is sometimes also important (FIG. 2). For example, PPR codons VTN/FTN/LTN are highly specific for A, while DTN preferentially recognizes U. Similarly, codon YNN specifically recognizes an A, while VNN prefers for C, and LNN and ENN have a preference for U.

Lastly, even the same codon can also have different specificity in different types of repeats (FIG. 2). One such example is VSN, which is much more specific for A in the P1-type of 36 aa (80.3%) than in the P1-type of 35 aa (45.9%). Similarly, VTD is more specific for G in the P1-type of 36 aa (90.8%) than in the P1-type of 35 aa (71.5%). Altogether, these data suggest the nuances of the PPR code and the importance of an unbiased approach to infer such a code from a large sample size.

The sequence context and scaffold.

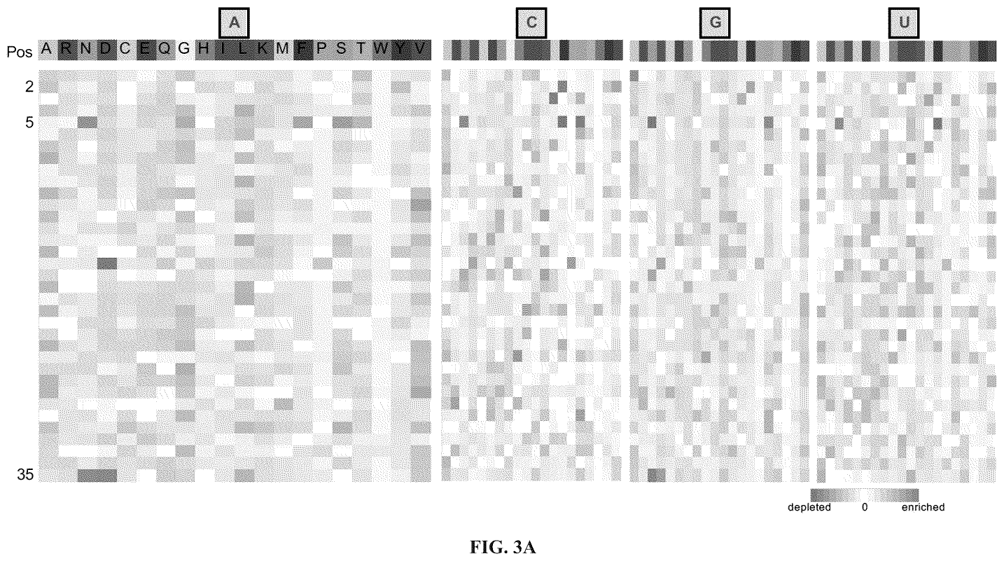

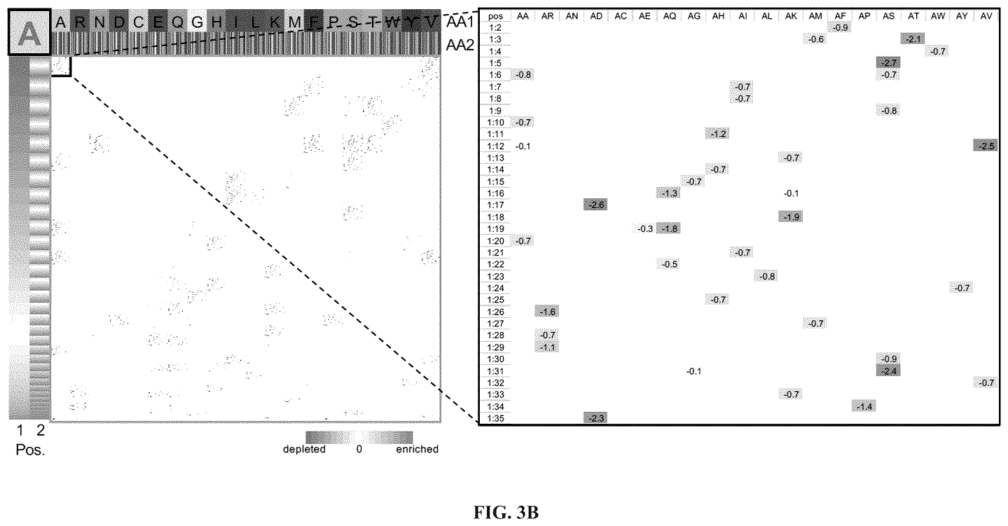

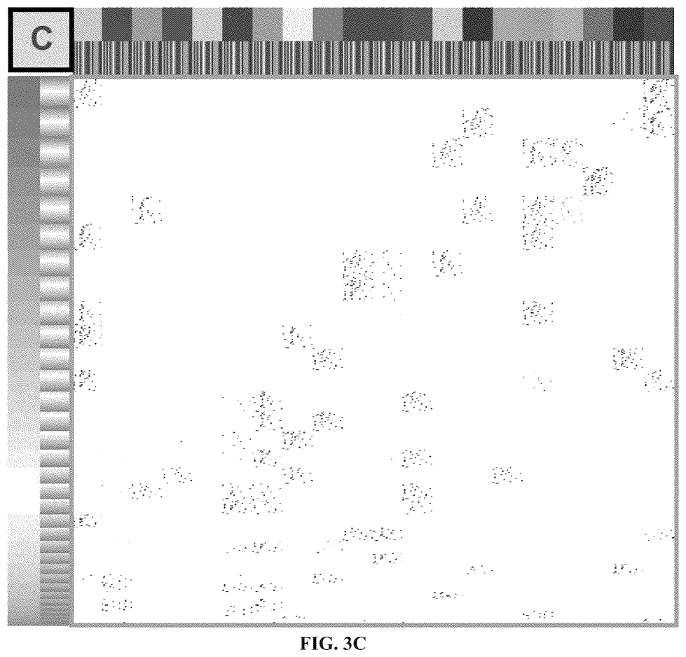

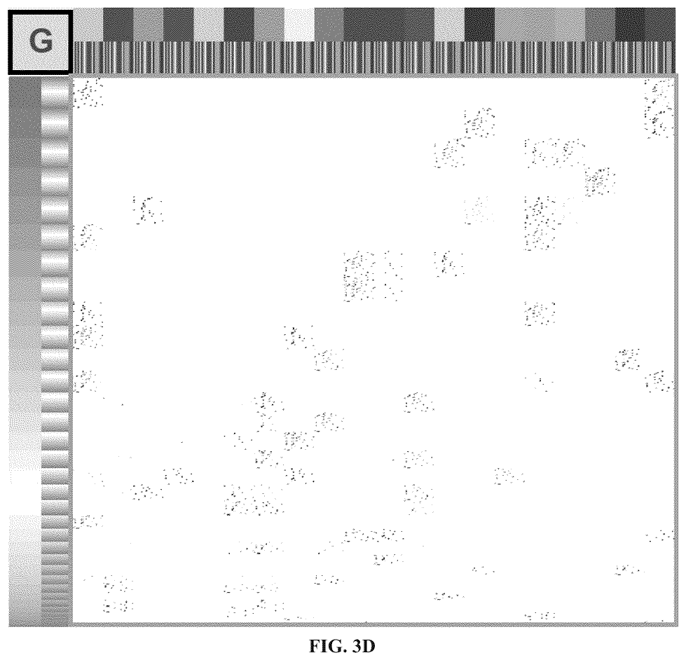

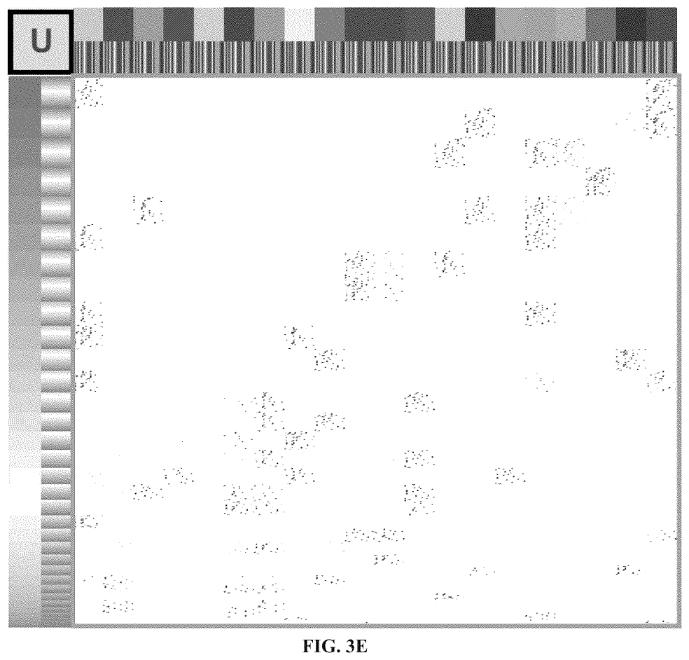

Previous structural analysis has identified additional amino acids other than positions 2, 5, and L contacting RNA (15). Whether amino acids in other positions contribute to binding specificity was investigated next. More specifically, whether the PPR motifs aligned with different nucleotide bases was examined to see whether there is any difference in the frequency of single amino acids at particular positions or amino acid combinations at particular pairs of positions (FIGS. 3A-3E). Surprisingly, in addition to the expected differences at positions 2, 5, and L, this analysis revealed many additional differences in single amino acids (FIG. 3A) or amino acid pairs (FIG. 3B-3E). It is somewhat difficult to envision that all these differences can directly contribute to binding specificity since a majority of the amino acids in the PPR motifs do not contact RNA. It was therefore conjectured that since PPR motifs are rapidly expanded during evolution, the observed association might be explained by dramatic and ununiform expansions of a relatively small number of particular repeats recognizing different nucleotide bases. If this is the case, phylogenetic analysis of PPR motifs might provide a means of clustering PPR proteins independent of the presence and type of the extension and the catalytic DYW domains.

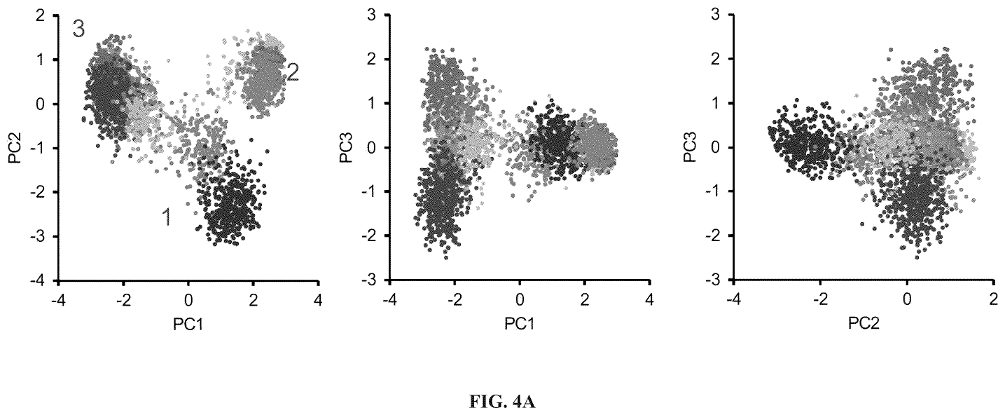

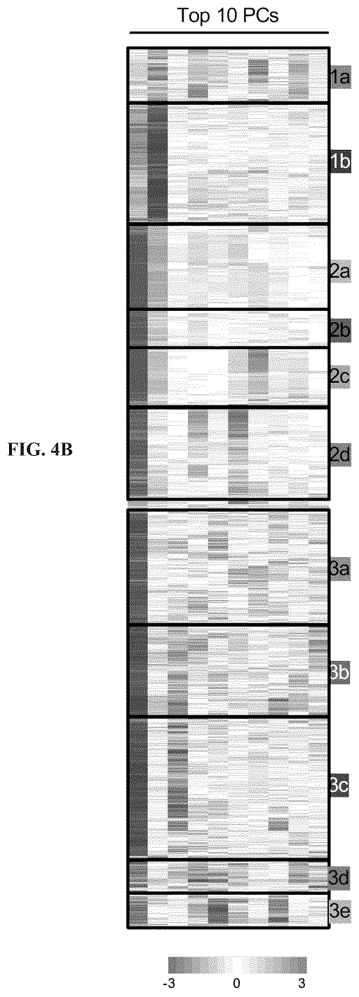

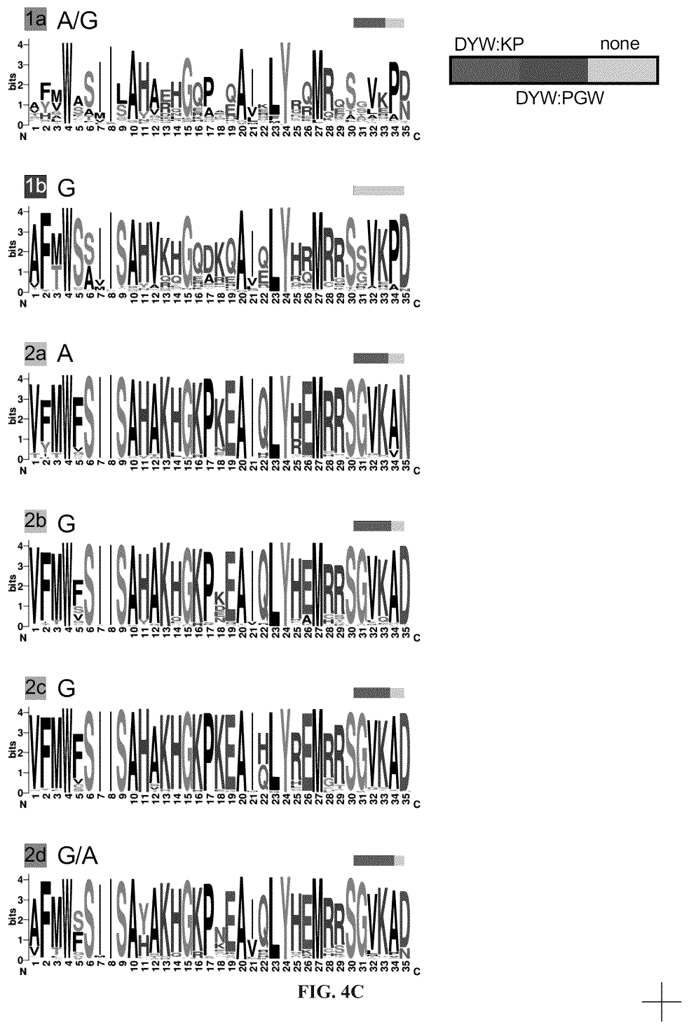

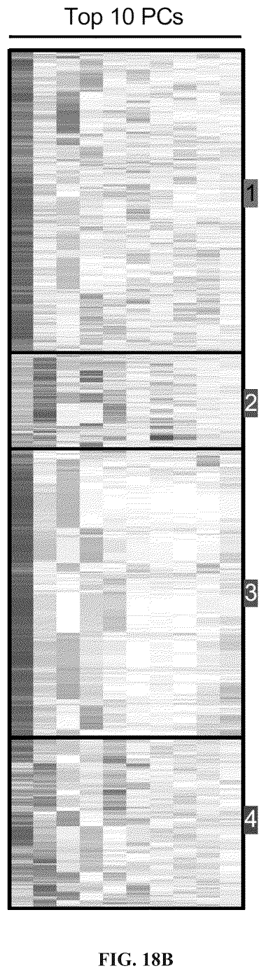

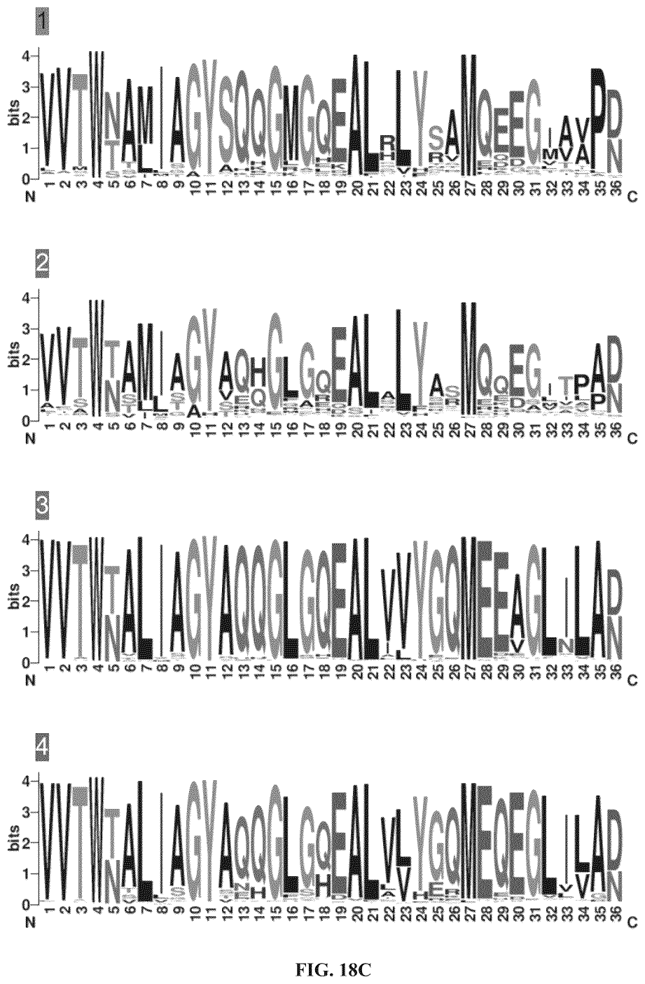

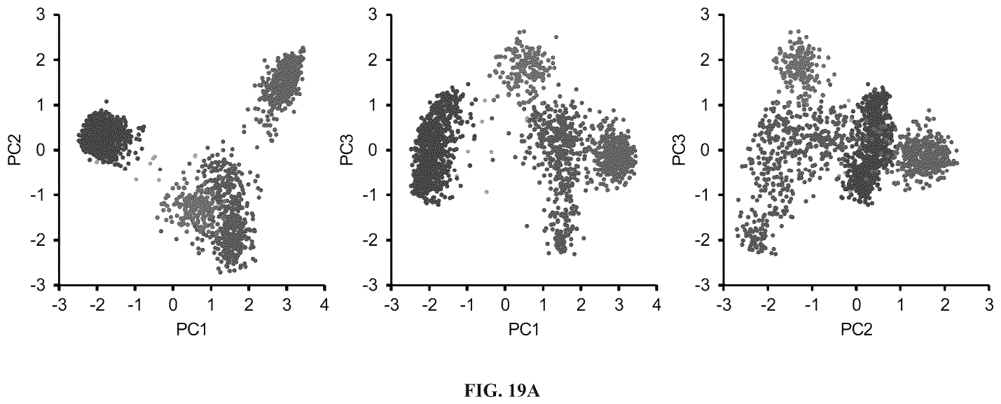

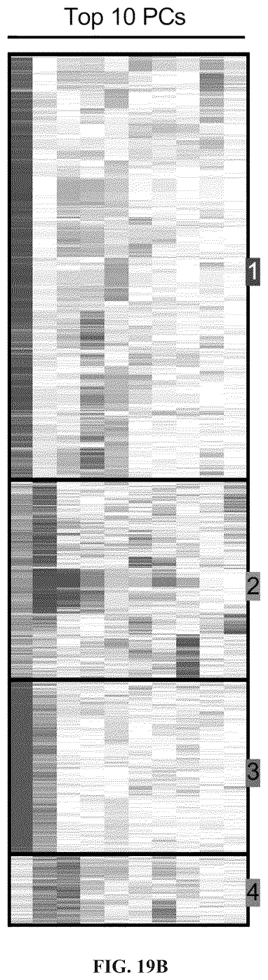

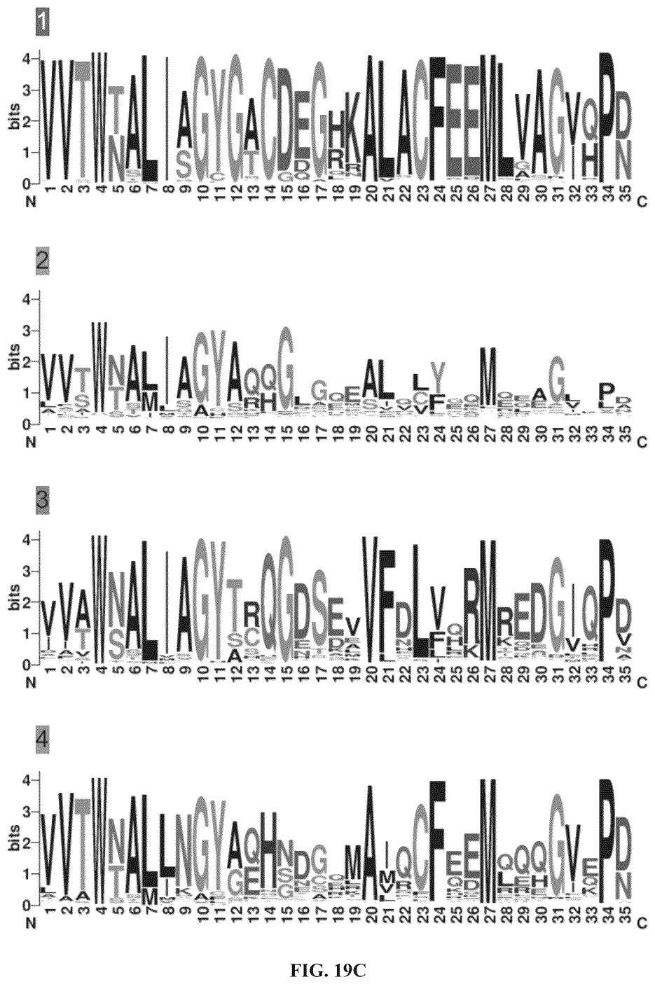

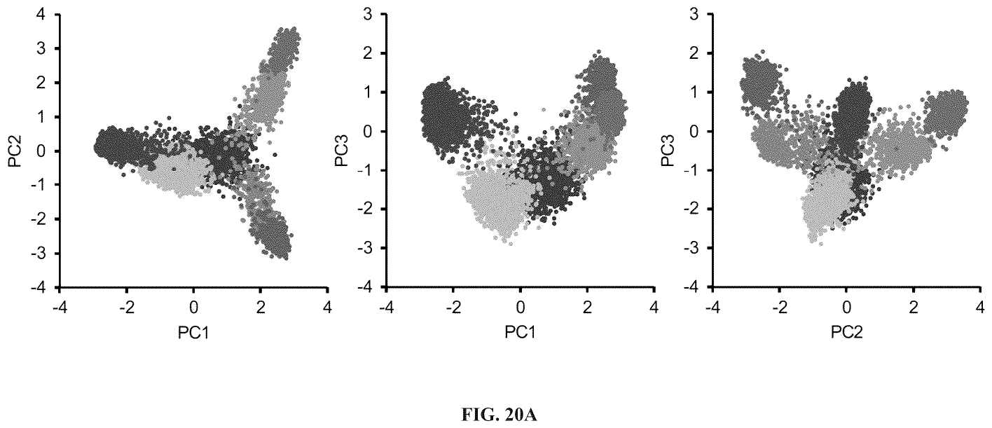

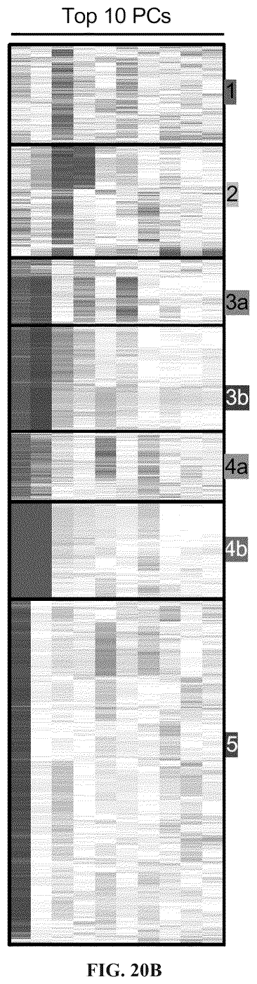

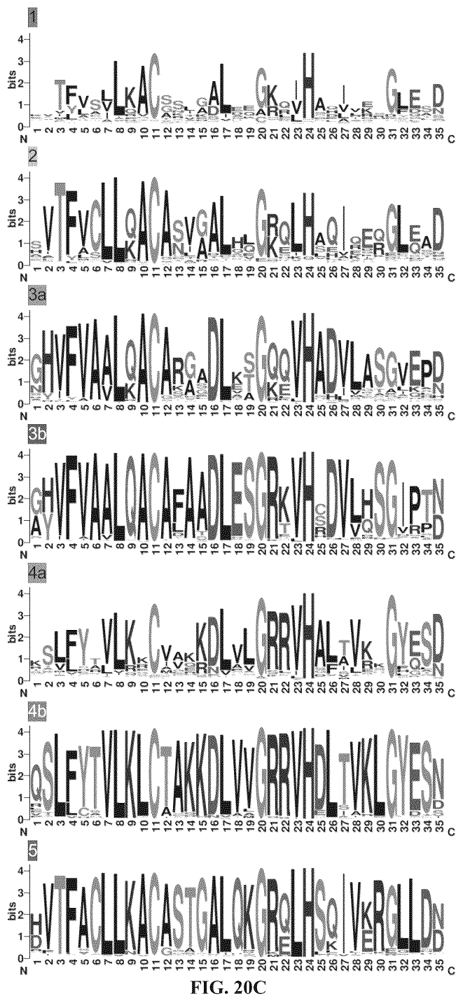

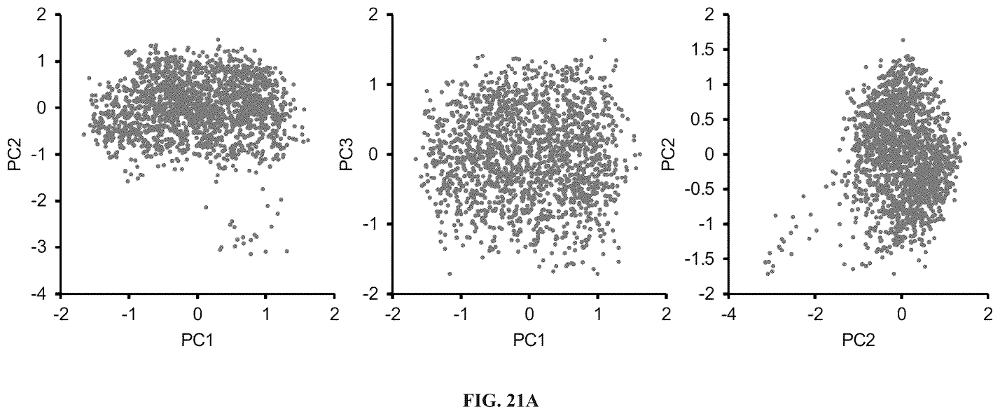

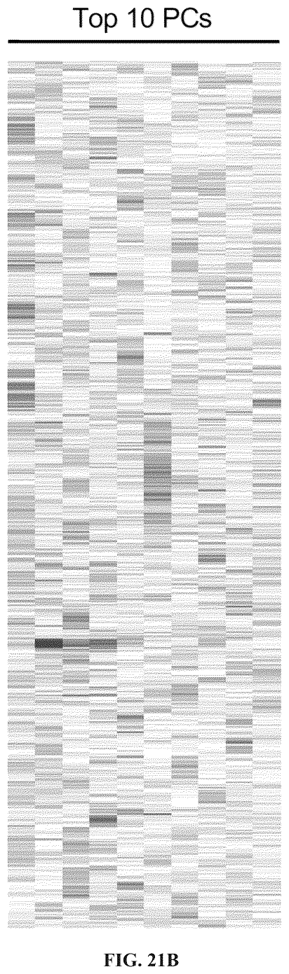

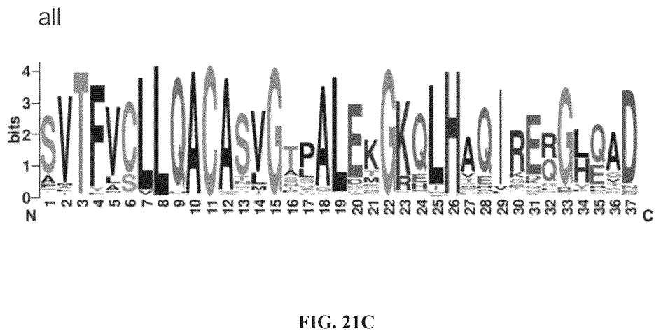

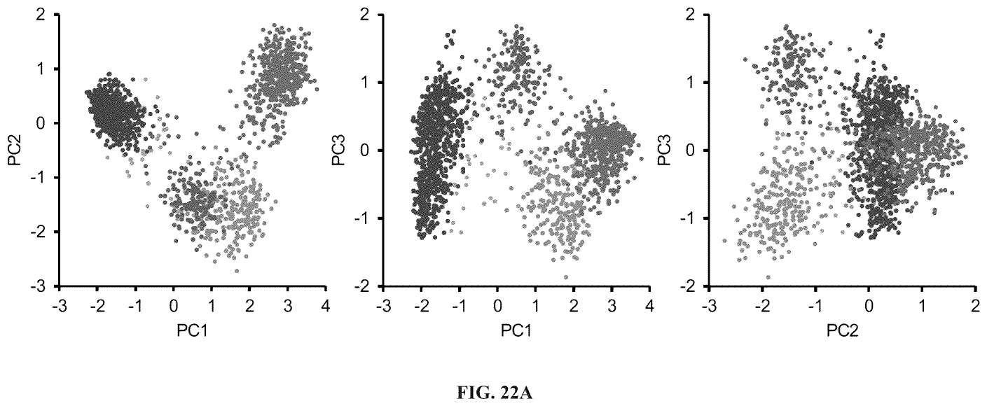

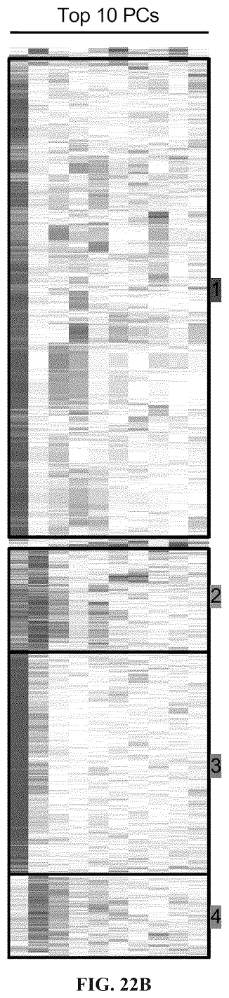

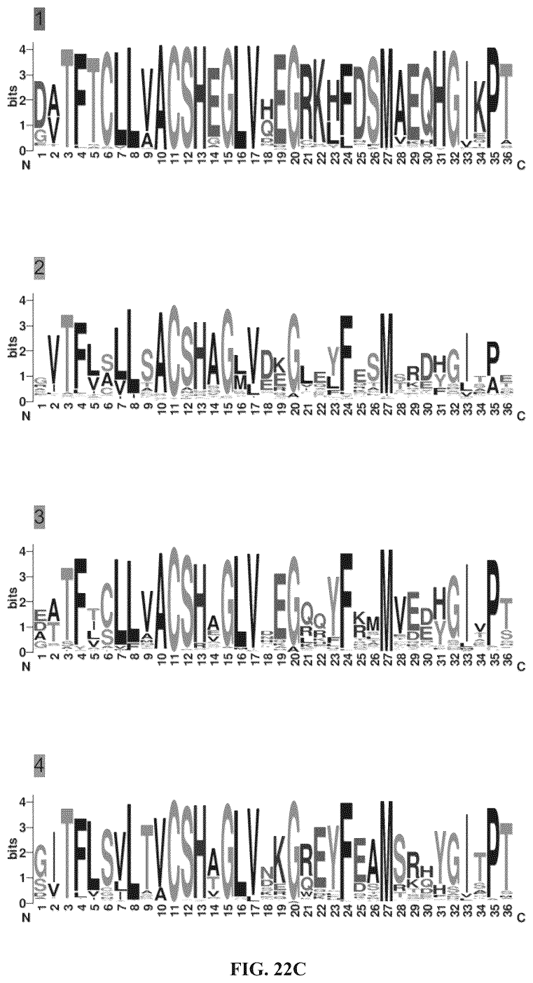

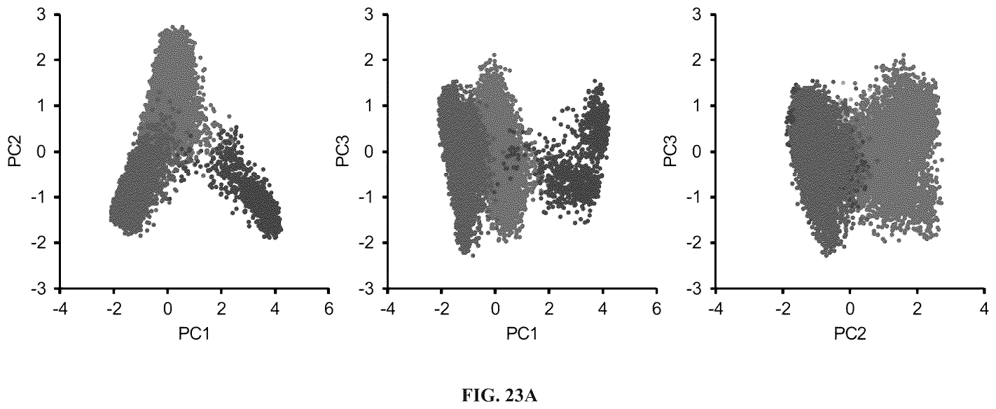

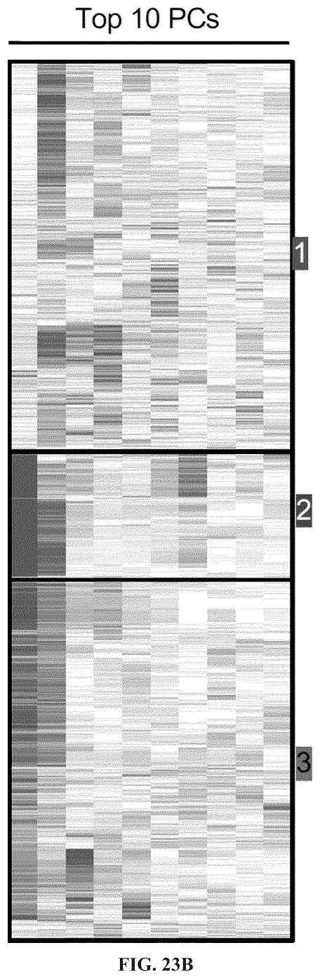

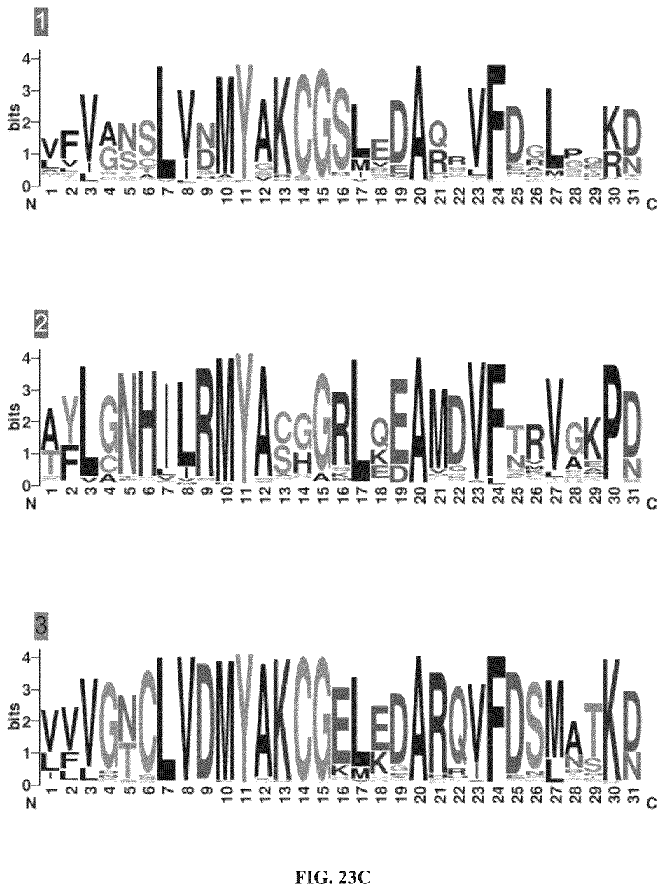

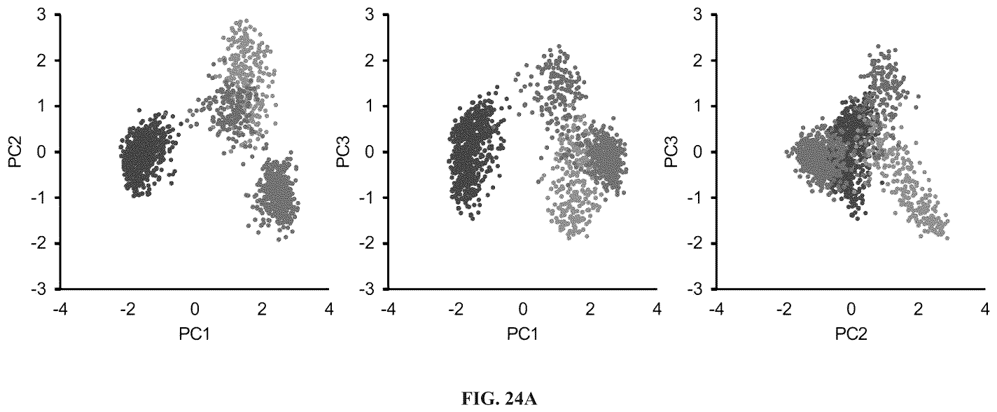

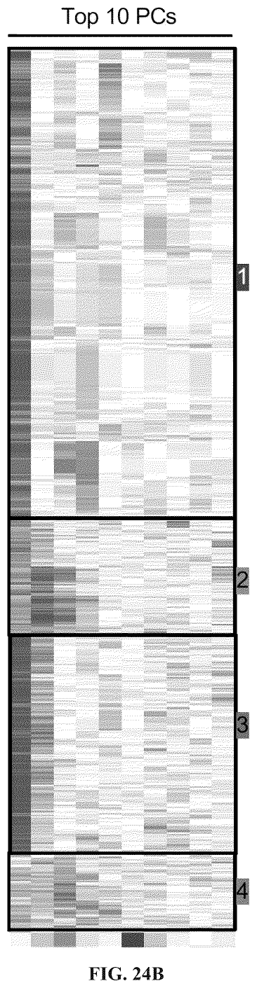

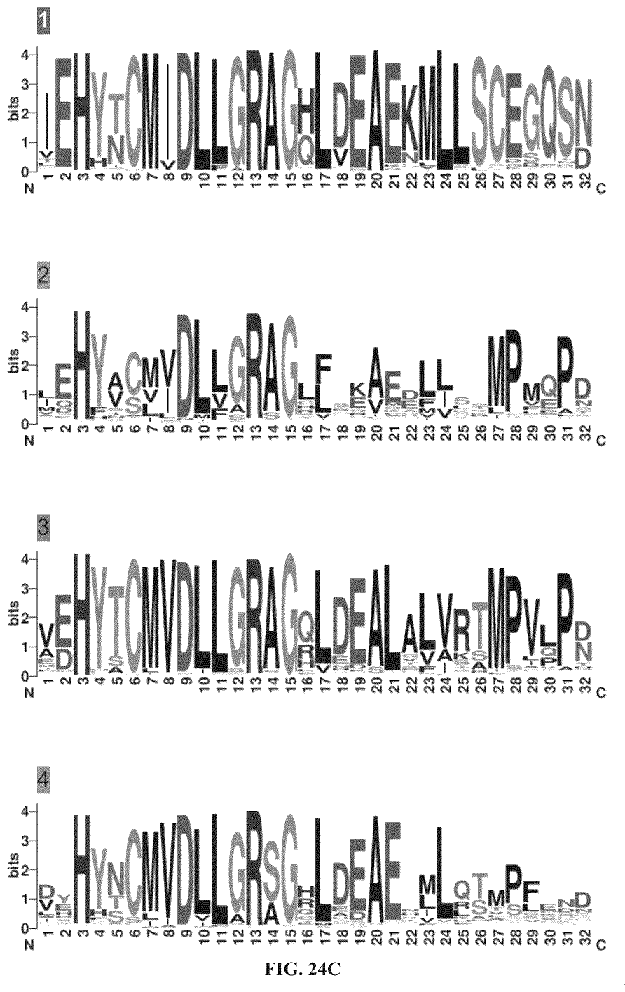

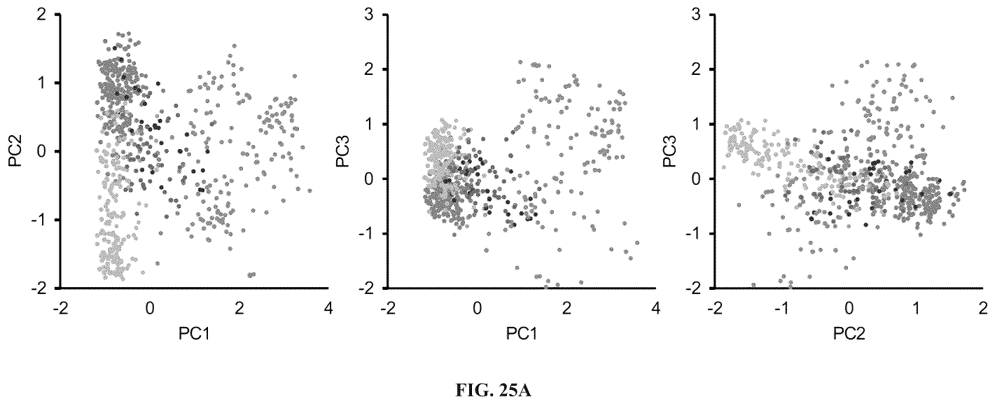

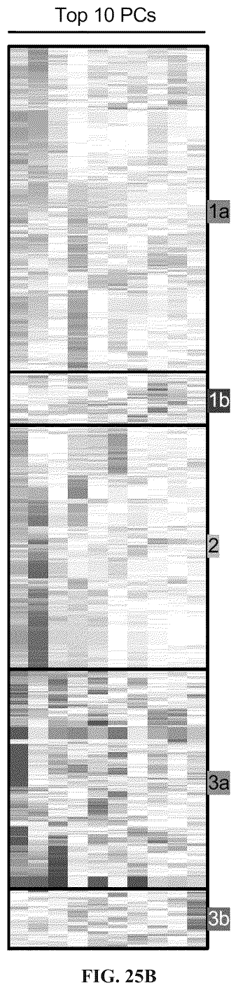

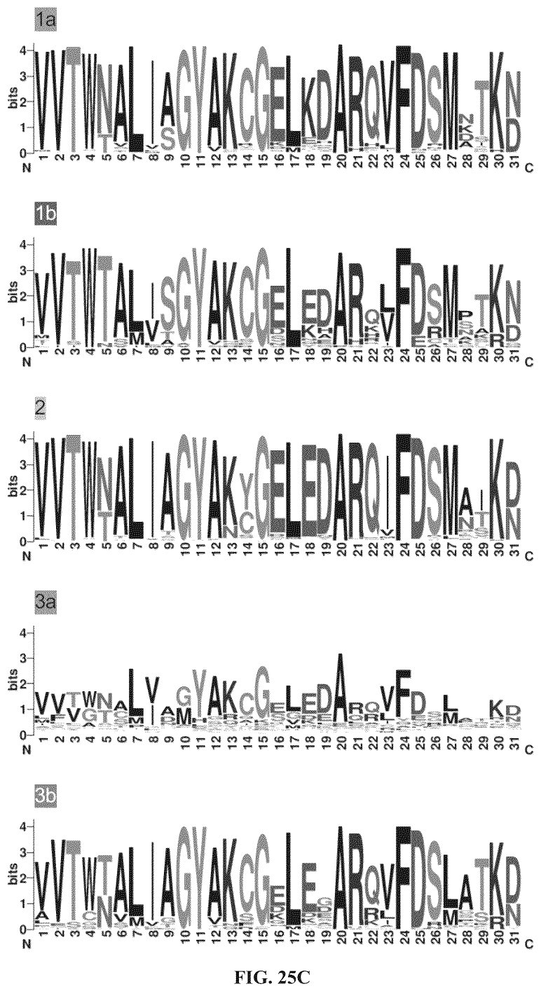

To test this hypothesis, a two-step approach was used to characterize PPR proteins while avoiding direct alignments of PPR proteins and their PPR arrays, which is challenging given the variation in the number and type of repeats. PPR motifs of particular types were analyzed and then lengths were analyzed separately by converting the amino acid sequence into a binary vector through one-hot encoding. Principal component analysis (PCA) was then performed to obtain a low-dimensional embedding of the repeats for data visualization and clustering. Distribution of PPR motifs in the low dimensional space along the top PCs showed clear clusters, which were formally identified by hierarchical clustering (FIGS. 4A, 4B, and 18 to 25). For example, for the P1-type repeats of 35 aa, 11 distinct clusters were identified, although the cluster number is somewhat arbitrary. Alignments of the amino acid sequences of the PPR motifs in each cluster revealed distinct consensuses (FIG. 2C). Several clusters, such as 2a-2d, showed a striking degree of amino acid conservation in a majority of positions, most likely due to a recent expansion, while other clusters (e.g., 1a, 3a-3e) showed more diversity. When PPR proteins were examined with the absence or presence of different types of DYW domains concerning which clusters their repeats belong to, particular repeat clusters were found to be uniquely represented in specific types of PPR proteins. For example, repeats in clusters 2a-2d are mostly found in PPRs with DYW:KP domains, while repeats in clusters 3a-3e are mostly found in PPR proteins with DYW:PGW domains or no detected DYW domains. Similar observations were observed for other repeat types, with one exception for the L1-type of 37 aa, for which no obvious clustering was observed (FIGS. 18-20, 22-25 vs. FIG. 21). In some cases, clear nucleotide base specificity was observed for particular clusters (e.g., cluster 1b, 2b, 2c for G and cluster 2a for A in FIG. 4C; and another example in FIG. 18), suggesting certain PCs captured variation in PPR codon amino acids. In total, PPR motifs of different types were grouped into 42 clusters.

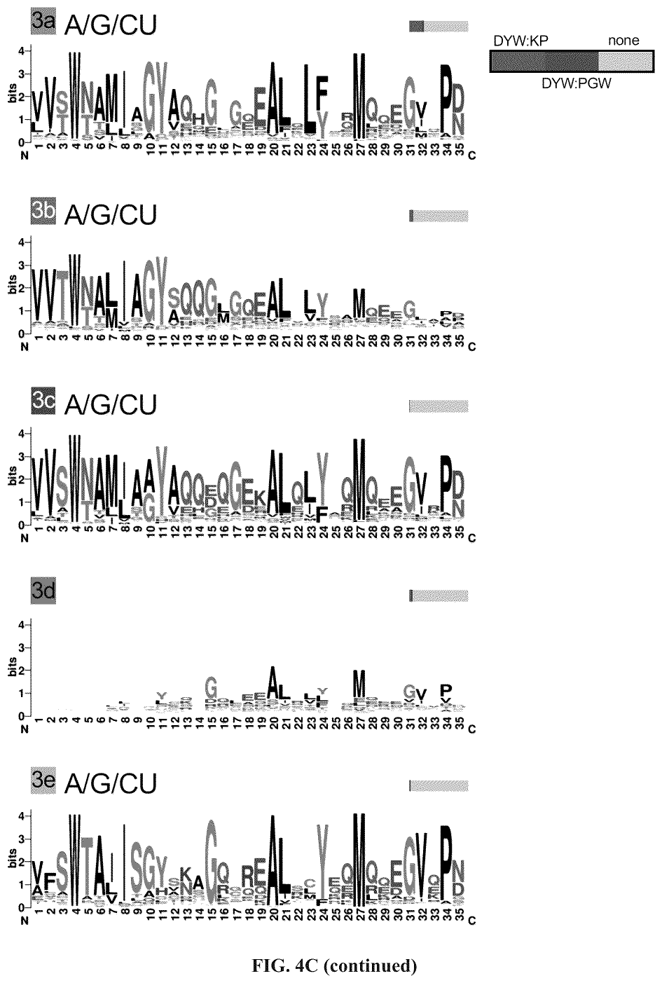

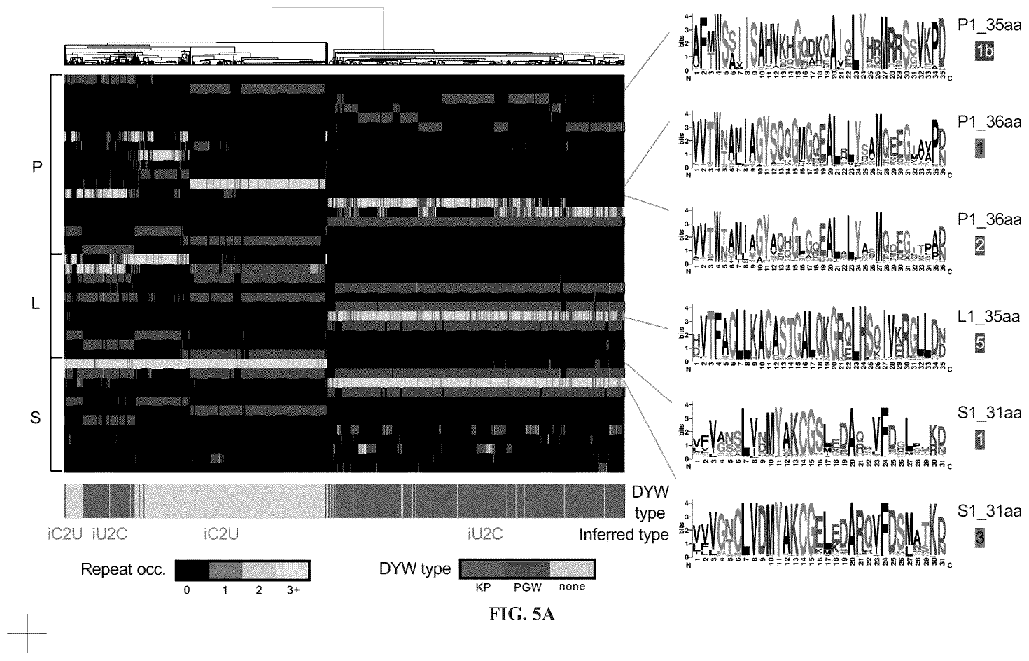

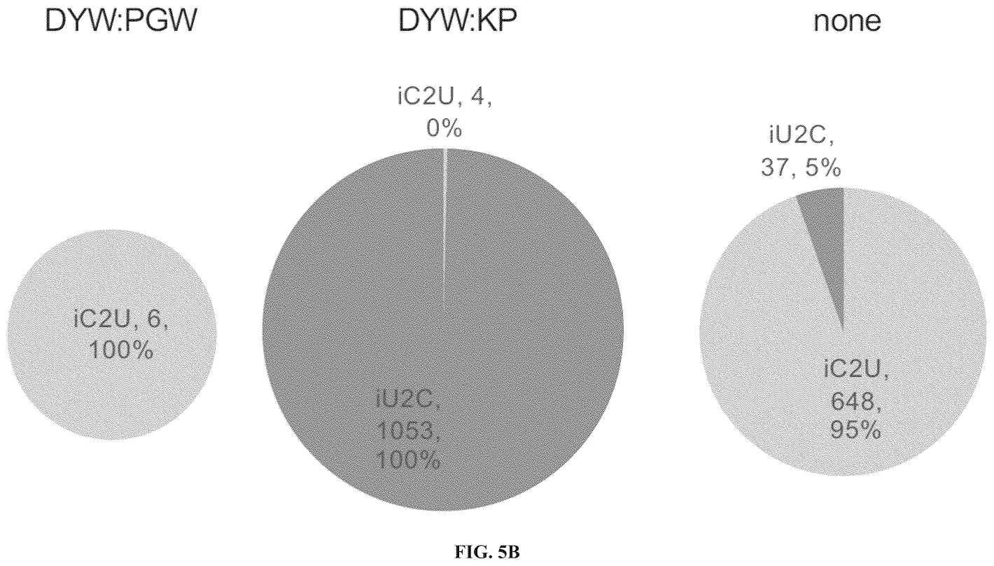

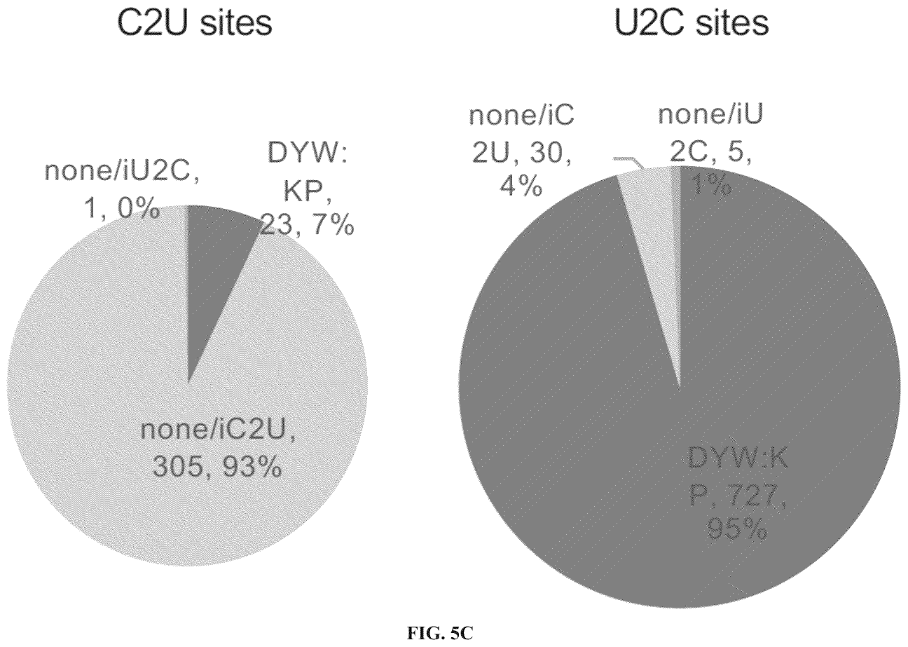

Next, each PPR protein was represented using the number of PPR motifs from each of the 42 clusters and another hierarchical clustering was performed to identify protein clusters. Examination of the presence and types of DYW domains, which were not used for clustering, revealed nearly perfect segregation of DYW:KP-containing PPRs in two clusters (FIG. 5A), while PPRs with classical DYW:PGW domain or without detected DYW domain are distributed in other clusters. Based on this observation, these clusters were assigned as inferred U-to-C (iU2C) editors or inferred C-to-U (iC2U) editors. All six PPRs with DYW:PGW domain were assigned as iC2U editors. All PPRs with DYW:KP except four proteins (99.6%) were assigned as iU2C editors. For 685 PPR proteins without a detected DYW domain, 95% were assigned as iC2U editors, while 37 (5%) were assigned as iU2C editors.

DYW domain annotations were then complemented with inferred editor types to re-examine how the types of editing sites match the types of PPR proteins. For C-to-U editing sites, 93% were iC2U editors, while 7% for matched proteins with DYW:KP or iU2C editors. For U-to-C editing sites, 96% were matched to proteins with DYW:KP domains or iU2C editors, and only 4% were matched to iC2U editors. Altogether, these data suggest the excellent concordance between the types of RNA editing sites and editor types, again supporting the accuracy of PPRDecoder.

### IV. Methods

### a) PPR Proteins and RNA Editing Site Compilation.

The list of 2,447 organelle C-to-U (C2U, 1,132 sites) or U-to-C (U2C, 1,057 sites) editing sites and editing levels were obtained from a previous study (9). The chloroplast and mitochondrial genomes of A. agrestis were downloaded from NCBI/GenBank (Accession: MK087646 and MK087647) and were used to extract the 54-nucleotide (nt) upstream flanking sequences (position −53 to 0, 0=editing site).

The list of PPR proteins together with their protein domain annotations in A. agrestis were kindly provided by Dr. Ian Small and predicted using PPRFinder as described previously (8, 9). The original list of 5,359 candidate PLStype PPR proteins was filtered by requiring ≥8 PPR motifs and the presence of the E1 domain; 1,748 proteins satisfying these criteria were used for this study. The presence of the E1 domain ensures the PPR array is complete on the C-termini, so that the last PPR motif aligns with position −4 relative to the editing site (18-20).

### b) the PPRDecoder Algorithm.

Previous studies aimed to infer the PPR code relied on a list of experimentally verified targets (18-20), which is very limited in number and may potentially be subject to ascertainment bias. PPRDecoder is a computational algorithm that takes comprehensive lists of PPR proteins and organelle editing sites to match PPRs with their target sites while statistically inferring the PPR code at the same time without requiring any experimental evidence of PPR-target pairing. For this study, the focus was on the PLS-type PPRs in A. agrestis, which are dramatically expanded during evolution, together with a large number of editing sites in the mitochondria and chloroplast transcriptomes, so that PPRDecoder can leverage RNA editing sites, which informs PPR binding sites, to limit the search space.

Here the PPR code refers to θ^{k}(b), the preference of each amino acid triplet at positions 2, 5, and L of PPR motifs, denoted PPR codon Cₖ (k=1, 2, . . . , 8000), for each nucleotide base b=A, C, G, and U.

### Objective Function.

Denote the collection of M target editing sites represented by upstream flanking sequences {B^{t}} indexed by t=1, 2, . . . , M. The objective of PPRDecoder is to find the optimal model parameters Θ={θ^{k}(b)} that maximize the likelihood function:

$$L ( Θ ) = P ( {B}^{1} , {B}^{2} , … , {B}^{M} \vert Θ ) \qquad ( 1 )$$

Denote the collection of N PPR proteins indexed by r=1, 2, . . . , N, and each PPR has W^{r} repeats indexed by i. The preference of each repeat i in PPR r for nucleotide base b is denoted

$${p}_{i}^{r} ( b ) ,$$

which is determined by the PPR code:

$${p}_{i}^{r} ( b ) = {\sum }_{k = 1}^{8000} {θ}^{k} ( b ) I ( {c}_{i}^{r} , {C}_{k} ) , where {c}_{i}^{r} \qquad ( 2 )$$

is the respective PPR codon, and

$$I ( {c}_{i}^{r} , {C}_{k} )$$

is the indicator function that equals to 1 when

$${c}_{i}^{r} = {C}_{k} ,$$

and 0 otherwise.

Denote each target site sequence

$${B}^{t} = {b}_{1}^{t} , {b}_{2}^{t} , … , {b}_{L}^{t} indexed by j ( {b}_{j}^{t} = A , C , G , U ) , in which {b}_{L}^{t}$$

is nucleotide at position −4 relative to the editing site k.

The probability of observing sequence B^{t} from background is

$${p}^{t \vert 0} = {\prod }_{j = 1}^{L} {p}^{0} ( {b}_{j}^{t} ) , \qquad ( 3 )$$

where p⁰(b) is the background nucleotide base composition.

The probability of observing sequence Bₜ as target of PPR protein r is

$${p}^{t \vert r} = {\prod }_{j = 1}^{L - {W}^{r}} {p}^{0} ( {b}_{j}^{t} ) {\prod }_{i = 1}^{{W}^{r}} {p}_{i}^{r} ( {b}_{L - {W}^{r} + i}^{t} ) , \qquad ( 4 )$$

$$where {b}_{L - {W}^{r} + i}^{t} ( i = 1 , 2 , … , {W}^{r} ) \text{"}$$

denotes the last W^{r} nucleotides in sequence B^{t} aligned to the PPR motifs, i.e., PPR binding site.

The log-likelihood ratio of observing sequence Bt as a target of PPR r over the background is thus

$${S}^{t \vert r} = l {r}^{t \vert r} = {\sum }_{i = 1}^{{W}^{r}} log ( \frac{{p}_{i}^{r} ( {b}_{L - {W}^{r} + i}^{t} )}{{p}^{0} ( {b}_{L - {W}^{r} + i}^{t} )} ) . \qquad ( 5 )$$

$$Denote$$

$${s}_{i}^{r} ( b ) = log ( \frac{{p}_{i}^{r} ( b )}{{p}^{0} ( b )} ) , \qquad ( 6 )$$

in which b=A, C, G, and U.

$${s}_{i}^{r} ( b )$$

is commonly known as the scoring matrix in studies of protein and nucleic acid interactions (29, 30).

S^{t|r} and p^{t|r} can be re-written as follows:

$${S}^{t \vert r} = {\sum }_{i = 1}^{{W}^{r}} {s}_{i}^{r} ( {b}_{L - {W}^{r} + i}^{t} ) , \qquad ( 7 )$$

$${p}^{t \vert r} = exp ( {S}^{t \vert r} ) {p}^{t \vert 0} . \qquad ( 8 )$$

Thus, the likelihood function can be rewritten as follows:

$$L ( Θ ) = {\prod }_{t = 1}^{M} P ( {B}_{t} \vert θ ) = {\prod }_{t = 1}^{M} {\sum }_{r = 1}^{N} {p}^{t , r} = {\prod }_{t = 1}^{M} {\sum }_{r = 1}^{N} {p}^{r} {p}^{t \vert r} , \qquad ( 9 )$$

in which the prior probability of PPR rp^{r}=1/N.

The optimization problem can be solved by an iterative expectation maximization (EM) algorithm (28), as described below.

### c) Initialization.

The background base composition was estimated from flanking 46-nt sequences upstream of the editing sites (positions −49 to −4).

The initial base preference for each PPR codon was assigned based on weights obtained from ref. (9). These weights were determined empirically based on PPR motif type and amino acid identities at positions 5 and L, based on experimentally determined targets and insights from structural analysis of PPR-RNA complexes, as listed below. All unspecified codons for P or S types and all codons for L types were assigned the background base composition.

TABLE 1

| PPR-Type | Pos. 5 | Pos. L | A | C | G | U |
|---|---|---|---|---|---|---|
| P or S | T\|S | N | 0.9 | 0 | 0.1 | 0 |
| P or S | T\|S | D | 0.1 | 0 | 0.9 | 0 |
| P or S | T\|S | Not (N\|D) | 0.5 | 0 | 0.5 | 0 |
| P or S | N | N\|S | 0 | 0.6 | 0 | 0.4 |
| P or S | N | D | 0 | 0.3 | 0 | 0.7 |
| | N | Not | 0 | 0.5 | 0 | 0.5 |
| | | (N\|D\|S) | | | | |

| | | | | |
|---|---|---|---|---|
| All others (same as background) | 0.29 | 0.15 | 0.21 | 0.35 |

These probabilities were used to calculate the initial scoring matrix

$${\hat{s}}_{i}^{r} ( b )$$

(eq. 6), which were close, but not exactly the same as the weights used by the previous study (9). E step.

Given the initial PPR code and hence the scoring matrices of all PPR proteins, each sequence t can be scored with respect to PPR r based on the last W^{r} nucleotides aligned to PPR motifs using the scoring matrix:

$${\hat{S}}^{t \vert r} = {\sum }_{i = 1}^{{W}^{r}} {\hat{s}}_{i}^{r} ( {b}_{L - {W}^{r} + i}^{t} ) . \qquad ( 10 )$$

The posterior probability of sequence B^{t} being a target of PPR r is:

$${\hat{α}}^{r \vert t} = \frac{{\hat{p}}^{t \vert r} {p}^{r}}{p ( t )} \qquad ( 11 )$$

The list of PPRs predicted from the genome is expected to be relatively complete, while the comprehensiveness of the list of editing sites is less certain, especially for genes with low expression. Therefore it was assumed that each editing site is regulated by one and only one PPR protein, while the number of target editing sites for each PPR can vary from 0, 1, or multiple sites. With this assumption,

$${\sum }_{r = 1}^{N} {\hat{α}}^{r \vert t} = 1$$

for each site t, so that it can be estimated

$${\hat{α}}^{r \vert t} = \frac{exp ( {\hat{s}}^{t \vert r} )}{{\sum }_{v = 1}^{N} exp ( {\hat{s}}^{t \vert v} )} . \qquad ( 12 )$$

Each sequence B^{t} is assigned to PPR r with a probability â^{r|t}, so the total number of sequences assigned to PPR r can be readily estimated by

$${\hat{n}}^{r} = {\sum }_{t = 1}^{M} {\hat{α}}^{r \vert t} . \qquad ( 13 )$$

Importantly, the total number of nucleotide base b probabilistically assigned to repeat i of PPR r is

$${\hat{β}}_{i}^{r} ( b ) = {\sum }_{t = 1}^{M} {\hat{α}}^{r \vert t} I ( {b}_{L - {W}^{r} + i}^{t} , b ) , \qquad ( 14 )$$

The total number of nucleotide b assigned to codon Cₖ can be estimated by

$${\hat{γ}}^{k} ( b ) = {\sum }_{r = 1}^{N} {\sum }_{i = 1}^{{W}^{r}} {\hat{β}}_{i}^{r} ( b ) I ( {c}_{i}^{r} , {C}_{k} ) . \qquad ( 15 )$$

M step.

The model parameters can be updated with latent variables estimated in the E-step above:

$${\hat{θ}}^{k} ( b ) = \frac{{\hat{γ}}^{k} ( b )}{( {\hat{γ}}^{k} ( A ) + {\hat{γ}}^{k} ( C ) + {\hat{γ}}^{k} ( G ) + {\hat{γ}}^{k} ( U ) )} . \qquad ( 16 )$$

The scoring matrix for PPR r,

$${\hat{s}}_{i}^{r} ( b ) ,$$

can be updated accordingly using eqs. (2) and (6) above.

### d) Practical Considerations.

PPRDecoder allows each type of PPR motifs, as well as PPR motifs with different lengths, to have a different code. Specifically, it estimates the PPR code separately for PPR motifs of type P1 (35 aa), P1 (36 aa), P2 (35 aa), L1 (35 aa), L1 (37 aa), S1 (31 aa), S2 (32 aa), and SS (32 aa). PPR motif types of other lengths each have ≤50 instances across all predicted PPR proteins, so a non-informative code (i.e., background base composition) is used for them.

In addition, particular attention is paid to dealing with potential issues due to the small sample size to increase the robustness of the algorithm. In case the cognate PPRs of certain editing sites were not included in our list, a site is included to update model parameters in the EM procedure only if the predicted binding score of the best matched PPR protein is ≥8 (eqs. 12-14).

In addition, when the PPR code is updated using eq. (16), PPRDecoder uses a pseudocount 10 for the variance stabilization:

$${\hat{θ}}^{k} ( b ) = \frac{{\hat{γ}}^{k} ( b ) + 10 {p}^{0} ( b )}{( {\hat{γ}}^{k} ( A ) + {\hat{γ}}^{k} ( C ) + {\hat{γ}}^{k} ( G ) + {\hat{γ}}^{k} ( U ) + 10 )} . \qquad ( 17 )$$

To monitor the convergence of the EM procedure, PPRDecoder uses the total best match score defined as

$$TS = {\sum }_{t = 1}^{M} {max}_{r = 1 , 2 … , N} {\hat{S}}^{t \vert r} . \qquad ( 18 )$$

The EM procedure is terminated when the change in TS is ≤1e-4, when the assignment of best matches does not change anymore in the dataset.

Table 2-10 summarize the complete list of the PPR code inferred by PPRDecoder as shown in FIG. 2. The PPR codons shown in FIG. 2 are: VTN, VSN, VVN, YFN, FFN, YNN, FTN, LTN, FSN, ETN, ATT, FAN, HVN, SCN, SYN, SYS, VAN, YVN, VNN, FNN, FNS, FNT, YNS, VNS, VNT, VTD, VSD, FSD, FTD, VAD, FFD, YSD, FGD, LGD, LSD, LTD, ETD, FAD, HVD, VND, VLD, VVD, LNN, AND, SND, MND, LND, IND, TND, FND, YND, DTN, ENN, ILT, VLT, SYD, YVD, and VTT.

TABLE 2
The PPR code inferred for the P1 motif (35 amino
acids). PPR codons not listed in the table were
found to not have a nucleotide-base preference.

| 25 L | A | C | G | T/U |
|---|---|---|---|---|
| VTN | 0.80230266 | 0.03320346 | 0.06979606 | 0.09469782 |
| YFN | 0.83653377 | 0.0393305 | 0.03798774 | 0.08614798 |
| FFN | 0.92125549 | 0.01135841 | 0.02531783 | 0.04206827 |
| VNN | 0.04548341 | 0.70032897 | 0.0115705 | 0.24261711 |
| VTD | 0.0654959 | 0.11213478 | 0.71526958 | 0.10709973 |
| FSD | 0.03767907 | 0.00637939 | 0.8945566 | 0.06138494 |
| FTD | 0.12835388 | 0.10012256 | 0.62526353 | 0.14626002 |
| FFD | 0.03217566 | 0.00998258 | 0.90739679 | 0.05044497 |
| YSD | 0.0686525 | 0.06523561 | 0.70163941 | 0.16447248 |
| VND | 0.02494702 | 0.40510988 | 0.0230407 | 0.5469024 |

TABLE 3
The PPR code inferred for the P1 motif (36 amino
acids). PPR codons not listed in the table were
found to not have a nucleotide-base preference.

| 25 L | A | C | G | T/U |
|---|---|---|---|---|
| VTN | 0.95144749 | 0.00691333 | 0.03335722 | 0.00828196 |
| VSN | 0.8034388 | 0.03307352 | 0.05222572 | 0.11126196 |
| VNN | 0.04264641 | 0.66953772 | 0.01177355 | 0.27604231 |
| VNS | 0.05502761 | 0.74939804 | 0.03154297 | 0.16403139 |
| VNT | 0.071884 | 0.59277204 | 0.04322297 | 0.29212099 |
| VTD | 0.04711288 | 0.00676991 | 0.90843278 | 0.03768442 |
| VSD | 0.10393922 | 0.03984387 | 0.6899212 | 0.16629571 |
| VND | 0.02191325 | 0.19180504 | 0.01016889 | 0.77611282 |

TABLE 4
The PPR code inferred for the P2 motif (35 amino
acids). PPR codons not listed in the table were
found to not have a nucleotide-base preference.

| 25 L | A | C | G | T/U |
|---|---|---|---|---|
| VTN | 0.90049988 | 0.02424731 | 0.01073243 | 0.06452038 |
| VNN | 0.04576371 | 0.55576821 | 0.01877623 | 0.37969186 |
| VTD | 0.09647955 | 0.01638617 | 0.85677741 | 0.03035687 |
| VSD | 0.12714491 | 0.06728437 | 0.7119872 | 0.09358352 |
| VND | 0.06427711 | 0.23714082 | 0.02240311 | 0.67617896 |

TABLE 5
The PPR code inferred for the L1 motif (35 amino
acids). PPR codons not listed in the table were
found to not have a nucleotide-base preference.

| 25 L | A | C | G | T/U |
|---|---|---|---|---|
| VSN | 0.66396429 | 0.07636183 | 0.08203756 | 0.17763632 |
| VVN | 0.54331615 | 0.09201868 | 0.05641085 | 0.30825432 |
| FAN | 0.79832129 | 0.03459152 | 0.09154715 | 0.07554005 |
| HVN | 0.86343111 | 0.00776033 | 0.02041998 | 0.10838859 |
| SCN | 0.40760242 | 0.10660027 | 0.23914579 | 0.24665153 |
| SYN | 0.35856047 | 0.1415243 | 0.24310299 | 0.25681224 |
| SYS | 0.65403142 | 0.09507045 | 0.10454546 | 0.14635266 |
| VAN | 0.47961228 | 0.14979777 | 0.0438376 | 0.32675236 |
| YVN | 0.69880789 | 0.01331312 | 0.01582455 | 0.27205444 |
| VSD | 0.14964624 | 0.0835562 | 0.52841732 | 0.23838024 |
| VAD | 0.14553978 | 0.16933868 | 0.3269606 | 0.35816094 |
| FAD | 0.12820404 | 0.02952097 | 0.722345 | 0.11992999 |
| HVD | 0.14926946 | 0.01157021 | 0.51722736 | 0.32193297 |
| VLD | 0.1647513 | 0.28399219 | 0.04941495 | 0.50184156 |
| VVD | 0.223683 | 0.16597749 | 0.10287059 | 0.50746893 |
| SYD | 0.31166924 | 0.13327118 | 0.08183437 | 0.47322521 |
| YVD | 0.20985275 | 0.00789634 | 0.18962084 | 0.59263007 |

TABLE 6
The PPR code inferred for the L1 motif (37 amino
acids). PPR codons not listed in the table were
found to not have a nucleotide-base preference.

| 25 L | A | C | G | T/U |
|---|---|---|---|---|
| VVN | 0.44794348 | 0.24681842 | 0.05517286 | 0.25006524 |
| VAD | 0.23272754 | 0.07864238 | 0.24987712 | 0.43875296 |
| VLD | 0.11516365 | 0.22446741 | 0.08199978 | 0.57836916 |
| VVD | 0.292689 | 0.10393764 | 0.10052451 | 0.50284885 |

TABLE 7
The PPR code inferred for the L2 motif (36 amino
acids). PPR codons not listed in the table were
found to not have a nucleotide-base preference.

| 25 L | A | C | G | T/U |
|---|---|---|---|---|
| ATT | 0.3738359 | 0.16694742 | 0.10366807 | 0.35554861 |
| ILT | 0.20269584 | 0.16180955 | 0.10507043 | 0.53042418 |
| VLT | 0.18458689 | 0.1239047 | 0.06382799 | 0.62768043 |
| SYD | | | | |
| YVD | | | | |
| VTT | 0.23754359 | 0.19514427 | 0.13185166 | 0.43546048 |

TABLE 8
The PPR code inferred for the S1 motif (31 amino
acids). PPR codons not listed in the table were
found to not have a nucleotide-base preference.

| 25 L | A | C | G | T |
|---|---|---|---|---|
| VTN | 0.93805257 | 0.01118093 | 0.01827539 | 0.03249111 |
| VSN | 0.72895389 | 0.08495834 | 0.06464598 | 0.12144179 |
| YNN | 0.88730238 | 0.01845173 | 0.01605078 | 0.0781951 |
| FTN | 0.88516473 | 0.01454233 | 0.02495172 | 0.07534122 |
| LTN | 0.87897635 | 0.01319086 | 0.03062875 | 0.07720404 |
| FSN | 0.87715563 | 0.0075683 | 0.05961479 | 0.05566128 |
| VNN | 0.0551027 | 0.62318248 | 0.06197773 | 0.25973709 |
| FNN | 0.32615382 | 0.34020888 | 0.17758457 | 0.15605273 |
| FNS | 0.14930254 | 0.49048733 | 0.08392436 | 0.27628578 |
| FNT | 0.14189855 | 0.35841851 | 0.23017684 | 0.2695061 |
| YNS | 0.09956315 | 0.60237495 | 0.11955539 | 0.17850651 |
| VTD | 0.08366224 | 0.00587408 | 0.88340175 | 0.02706194 |
| VSD | 0.07619692 | 0.03867645 | 0.7688414 | 0.11628522 |
| FSD | 0.09306107 | 0.01581218 | 0.83570674 | 0.05542 |
| FTD | 0.08769467 | 0.01995582 | 0.84113175 | 0.05121775 |
| FGD | 0.18760124 | 0.09744948 | 0.54502116 | 0.16992812 |
| LGD | 0.18156233 | 0.07282832 | 0.54681855 | 0.1987908 |
| LSD | 0.13152022 | 0.12635364 | 0.49757585 | 0.24455029 |
| LTD | 0.09073024 | 0.02003175 | 0.83482863 | 0.05440938 |
| VND | 0.04448156 | 0.12218267 | 0.03443493 | 0.79890084 |
| LNN | 0.18292748 | 0.26573856 | 0.13875604 | 0.41257792 |
| AND | 0.16308794 | 0.14286915 | 0.1530801 | 0.54096281 |
| SND | 0.15381628 | 0.18759176 | 0.08231041 | 0.57628154 |
| MND | 0.11885963 | 0.18375989 | 0.08325516 | 0.61412532 |
| LND | 0.08446512 | 0.10038904 | 0.00907838 | 0.80606746 |
| IND | 0.07060174 | 0.1535377 | 0.09134452 | 0.68451604 |
| TND | 0.0696385 | 0.22082447 | 0.10800818 | 0.60152885 |
| FND | 0.05758986 | 0.08149165 | 0.03837762 | 0.82254087 |
| YND | 0.0438884 | 0.09226366 | 0.02478634 | 0.8390616 |

TABLE 9
The PPR code inferred for the S2 motif (32 amino
acids). PPR codons not listed in the table were
found to not have a nucleotide-base preference.

| 25 L | A | C | G | T/U |
|---|---|---|---|---|
| ETN | 0.55461394 | 0.04005702 | 0.22270698 | 0.18262205 |
| ETD | 0.06793576 | 0.00725233 | 0.82407066 | 0.10074124 |
| DTN | 0.18692173 | 0.06782514 | 0.04704749 | 0.69820564 |
| ENN | 0.03663834 | 0.30411974 | 0.00891689 | 0.65032502 |

TABLE 10
The PPR code inferred for the SS motif (31 amino
acids). PPR codons not listed in the table were
found to not have a nucleotide-base preference.

| 25 L | A | C | G | T/U |
|---|---|---|---|---|
| VTN | 0.91832322 | 0.02134879 | 0.02189005 | 0.03843794 |
| VNN | 0.04569289 | 0.69660418 | 0.01959277 | 0.23811015 |
| VTD | 0.07787599 | 0.01689612 | 0.84411436 | 0.06111352 |
| VND | 0.03252853 | 0.15385068 | 0.02737096 | 0.78624983 |

### e) Cumulative Run Test of Predicted DYW:KP-Containing PPRs.

To quantify the enrichment of U-to-C editing sites among top-scoring DYW:KP-containing PPRs, a cumulative run test was performed. Denote N1 and N2 are the number of U-to-C and C-to-U editing sites that are matched to DYW:KP-containing PPRs and ranked based on the predicted binding score, n and r-n are the number of U-to-C and C-to-U editing sites with a rank≤r. The run statistic at rank r is defined as n/N₁−(r−n)/N₂ (FIG. 1E).

### f) Visualization and Clustering of PPR Motifs Using Low Dimensional Embeddings.

Comparative analysis of PPR proteins using multiple sequence alignments is challenging due to the repetitive nature of the PPR array and their evolutionary plasticity. To characterize similarities between PPR motifs, as well as between PPR proteins without relying on direct sequence alignment, a method was developed to embed PPR motifs in lower dimensions for data visualization and clustering. Specifically, one-hot encoding was used to represent each amino acid using a 20-dimension binary vector, so a PPR motif of length P is represented by a 20′P dimension vector. This representation was used to perform a principal component analysis (PCA) for all PPR motifs of a particular type and length (e.g., P1 type of 35 aa; the same as the section above). PPR motifs that contain stop codons were excluded from this analysis.

Visual examination of PPR motifs using the first few principal components (PCs) revealed clear clusters (FIGS. 18-25). To formally define these clusters, centroid-linkage hierarchical clustering was performed using the top 10 PCs and Pearson correlation as the distance metric. Clusters were identified by visually examining the endrogram; a total of 42 clusters were identified when all PPR motif types were analyzed in this manner (FIGS. 18-25). Each PPR protein was then represented by a vector containing the number of PPR motifs that belong to each of the 42 clusters. This representation was used to identify protein clusters by centroid-linkage hierarchical clustering using Spearman rank correlation as the distance metric. The presence and types of DYW domains in the identified clusters were examined. Two clusters were almost exclusively PPRs with a DYW:KP domain, and thus inferred as U-to-C (iU2C) RNA editors, while the other clusters devoid of DYW:KP domain were inferred as C-to-U (iC2U) editors (FIG. 5A; left panel).

### REFERENCES CITED AND INCORPORATED BY REFERENCE

1. D. D. Licatalosi, R. B. Darnell, RNA processing and its regulation: global insights into biological networks. Nat Rev Genet 11, 75 (2010).
2. S. Bajan, G. Hutvagner, RNA-based therapeutics: from antisense oligonucleotides to miRNAs. Cells 9, (2020).
3. B. M. Lunde, C. Moore, G. Varani, RNA-binding proteins: modular design for efficient function. Nat Rev Mol Cell Biol 8, 479 (2007).
4. A. Filipovska, O. Rackham, Modular recognition of nucleic acids by PUF, TALE and PPR proteins. Mol Biosyst 8, 699 (2012).
5. A. Barkan, I. Small, Pentatricopeptide repeat proteins in plants. Annu Rev Plant Biol 65, 415 (2014).
6. I. D. Small, N. Peeters, The PPR motif—a TPR-related motif prevalent in plant organellar proteins. Trends Biochem Sci 25, 46 (2000).
7. S. Aubourg, N. Boudet, M. Kreis, A. Lecharny, In Arabidopsis thaliana, 1% of the genome codes for a novel protein family unique to plants. Plant Mol Biol 42, 603 (2000).
8. B. Gutmann et al., The expansion and diversification of pentatricopeptide repeat RNA-editing factors in plants. Mol Plant 13, 215 (2020).
9. P. Gerke et al., Towards a plant model for enigmatic U-to-C RNA editing: the organelle genomes, transcriptomes, editomes and candidate RNA editing factors in the hornwort Anthoceros agrestis. New Phytol 225, 1974 (2020).
10. C. Lurin et al., Genome-wide analysis of Arabidopsis pentatricopeptide repeat proteins reveals their essential role in organelle biogenesis. Plant Cell 16, 2089 (2004).
11. J. Prikryl, M. Rojas, G. Schuster, A. Barkan, Mechanism of RNA stabilization and translational activation by a pentatricopeptide repeat protein. Proc Natl Acad Sci USA 108, 415 (2011).
12. E. Kotera, M. Tasaka, T. Shikanai, A pentatricopeptide repeat protein is essential for RNA editing in chloroplasts. Nature 433, 326 (2005).
13. C. Loiselay et al., Molecular identification and function of cis- and trans-acting determinants for petA transcript stability in Chlamydomonas reinhardtii chloroplasts. Mol Cell Biol 28, 5529 (2008).
14. S. Fujii, C. S. Bond, I. D. Small, Selection patterns on restorer-like genes reveal a conflict between nuclear and mitochondrial genomes throughout angiosperm evolution. Proc Natl Acad Sci USA 108, 1723 (2011).
15. C. Shen et al., Structural basis for specific single-stranded RNA recognition by designer pentatricopeptide repeat proteins. Nat Commun 7, 11285 (2016).
16. P. Yin et al., Structural basis for the modular recognition of single-stranded RNA by PPR proteins. Nature 504, 168 (2013).
17. K. Kobayashi et al., Identification and characterization of the RNA binding surface of the pentatricopeptide repeat protein. Nucleic Acids Res 40, 2712 (2012).
18. A. Barkan et al., A combinatorial amino acid code for RNA recognition by pentatricopeptide repeat proteins. PLoS Genet 8, e1002910 (2012).
19. Y. Yagi, S. Hayashi, K. Kobayashi, T. Hirayama, T. Nakamura, Elucidation of the RNA recognition code for pentatricopeptide repeat proteins involved in organelle RNA editing in plants. PLoS One 8, e57286 (2013).
20. M. Takenaka, A. Zehrmann, A. Brennicke, K. Graichen, Improved computational target site prediction for pentatricopeptide repeat RNA editing factors. PLoS One 8, e65343 (2013).
21. R. McDowell, I. Small, C. S. Bond, Synthetic PPR proteins as tools for sequence-specific targeting of RNA. Methods 208, 19 (2022).
22. B. S. Gully et al., The design and structural characterization of a synthetic pentatricopeptide repeat protein. Acta Crystallogr D Biol Crystallogr 71, 196 (2015).
23. M. Rojas, Q. Yu, R. Williams-Carrier, P. Maliga, A. Barkan, Engineered PPR proteins as inducible switches to activate the expression of chloroplast transgenes. Nat Plants 5, 505 (2019).
24. K. Bernath-Levin et al., Cofactor-independent RNA editing by a synthetic S-type PPR protein. Synth Biol (Oxf) 7, ysab034 (2021).
25. J. Yan et al., Delineation of pentatricopeptide repeat codes for target RNA prediction. Nucleic Acids Res 47, 3728 (2019).
26. S. Coquille et al., An artificial PPR scaffold for programmable RNA recognition. Nat Commun 5, 5729 (2014).
27. E. Lesch et al., Plant mitochondrial RNA editing factors can perform targeted C-to-U editing of nuclear transcripts in human cells. Nucleic Acids Res 50, 9966 (2022).
28. A. P. Dempster, N. M. Laird, D. B. Rubin, Maximum likelihood from incomplete data via the EM algorithm. J R Stat Soc Series B Stat Methodol 39, 1 (1977).
29. G. D. Stormo, DNA binding sites: representation and discovery. Bioinformatics 16, 16 (2000).
30. O. G. Berg, P. H. von Hippel, Selection of DNA binding sites by regulatory proteins. Statisticalmechanical theory and application to operators and promoters. J Mol Biol 193, 723 (1987).
31. C. Boussardon et al., Two interacting proteins are necessary for the editing of the NdhD-1 site in Arabidopsis plastids. Plant Cell 24, 3684 (2012).
32. M. Takenaka et al., Multiple organellar RNA editing factor (MORF) family proteins are required for RNA editing in mitochondria and plastids of plants. Proc Natl Acad Sci USA 109, 5104 (2012).
33. S. Bentolila et al., RIP1, a member of an Arabidopsis protein family, interacts with the protein RARE1 and broadly affects RNA editing. Proc Natl Acad Sci USA 109, E1453 (2012).

I/We claim:

1. A computational method for matching pentatricopeptide repeat (PPR) proteins with target sequences in an organism and inferring a PPR code, the method comprising:
receiving sequence data representing PPR editing sites in the organism and at least one PPR protein expressed in the organism;
estimating a background base composition for a PPR code from flanking 46-nucleotide sequences upstream of the editing sites (positions −49 to −4);
assigning an initial nucleotide base preference for each PPR codon of the at least one PPR protein based on nucleotide probability parameters to determine an initial PPR code predictive model;
calculating an initial scoring matrix for the initial PPR code predictive model;
updating the initial PPR code predictive model by:
using the initial scoring matrix to score each target sequence with respect to the at least one PPR protein;
assigning each target sequence to the at least one PPR protein with a probability;
estimating a total number of target sequences assigned to the at least one PPR protein;
estimating a total number of each nucleotide base assigned to each PPR codon of the at least one PPR protein based on the estimated total number of target sequences assigned to the at least one PPR protein;
updating the nucleotide probability parameters based on the estimated total number of each nucleotide base assigned to each PPR codon;
assigning an updated nucleotide base preference for each PPR codon based on the updated nucleotide probability parameters to determine an updated PPR code predictive model; and
calculating an updated scoring matrix for the updated PPR code predictive model;
iteratively updating the updated PPR code predictive model until a best match of a target sequence to the at least one PPR protein does not change any more, indicating a match between the at least one PPR protein and the target sequence; and
inferring the PPR code to be the most recent PPR code predictive model after the iteratively updating is complete.

2. The method of claim 1, further comprising determining a total best match score after each instance of updating the updated PPR code, wherein a change in the total best match score falling below a predetermined threshold indicates that the best match of a target sequence to the at least one PPR protein does not change any more.

3. The method of claim 1, wherein the at least one PPR protein comprises a PLS-type PPR protein.

4. The method of claim 1, wherein each PPR codon comprises an amino acid triplet of amino acids at a second position, fifth position, and last position of a PPR motif of the at least one PPR protein.

5. The method of claim 4, wherein the PPR code comprises a preference of the amino acid triplet of each PPR motif for each nucleotide base.

6. The method of claim 1, wherein the at least one PPR protein comprises a plurality of a single type of PPR motifs, or a plurality of different types of PPR motifs.

7. The method of claim 6, wherein the types of PPR motifs are selected from the group consisting of: P1, P2, L1, L2, S1, S2, and SS.

8. The method of claim 1, further comprising outputting a best matched PPR protein for each editing site.

9. The method of claim 1, wherein the method is carried out without any experimental evidence of PPR-target sequence pairing.

10. The method of claim 1, wherein the background base composition comprises a probability for nucleotide base A of 0.29, a probability for nucleotide base C of 0.15, a probability for nucleotide base G of 0.21, and a probability for nucleotide base U of 0.35.

11. The method of claim 1, wherein assigning the initial nucleotide base preference for each PPR codon is based on nucleotide probability parameters in the following table:

| PPR-Type | Pos. 5 | Pos. L | A | C | G | U |
|---|---|---|---|---|---|---|
| P or S | T\|S | N | 0.9 | 0 | 0.1 | 0 |
| P or S | T\|S | D | 0.1 | 0 | 0.9 | 0 |
| P or S | T\|S | Not (N\|D) | 0.5 | 0 | 0.5 | 0 |
| P or S | N | N\|S | 0 | 0.6 | 0 | 0.4 |
| P or S | N | D | 0 | 0.3 | 0 | 0.7 |
| | N | Not (N\|D\|S) | 0 | 0.5 | 0 | 0.5 |
| | | | | |
|---|---|---|---|---|
| All others (same as background) | 0.29 | 0.15 | 0.21 | 0.35 |

12. A computational method for matching pentatricopeptide repeat (PPR) proteins with target sequences in an organism and inferring a PPR code, the method comprising:
receiving sequence data representing PPR editing site in the organism and PPR proteins expressed in the organism;
estimating a background base composition for a PPR code;
assigning an initial nucleotide base preference for each PPR codon of the PPR proteins to determine an initial PPR code predictive model;
calculating an initial scoring matrix for the initial PPR code predictive model;
updating the initial PPR code predictive model by performing an iterative expectation-maximization procedure until a best match of a target sequence to a PPR protein does not change any more, indicating a match between the PPR protein and target sequence, wherein the target sequence comprises an editing site; and
inferring the PPR code to be the most recent PPR code predictive model after the updating is complete.

13. The method of claim 12, wherein estimating the background base composition for the PPR code is based on flanking 46-nucleotide sequences upstream of the editing sites (positions −49 to −4).

14. The method of claim 12, wherein assigning the initial nucleotide base preference for each PPR codon of the PPR proteins is based on nucleotide probability parameters derived from the following table:

| PPR-Type | Pos. 5 | Pos. L | A | C | G | U |
|---|---|---|---|---|---|---|
| P or S | T\|S | N | 0.9 | 0 | 0.1 | 0 |
| P or S | T\|S | D | 0.1 | 0 | 0.9 | 0 |
| P or S | T\|S | Not (N\|D) | 0.5 | 0 | 0.5 | 0 |
| P or S | N | N\|S | 0 | 0.6 | 0 | 0.4 |
| P or S | N | D | 0 | 0.3 | 0 | 0.7 |
| | N | Not (N\|D\|S) | 0 | 0.5 | 0 | 0.5 |
| | | | | |
|---|---|---|---|---|
| All others (same as background) | 0.29 | 0.15 | 0.21 | 0.35 |

15. A computational method for predicting whether an editing site is a site for U-to-C editing, the method comprising:
receiving sequencing data representing PPR editing site in the organism and PPR proteins expressed in the organism;
estimating a background base composition for a PPR code;
assigning an initial nucleotide base preference for each PPR codon of the PPR proteins to determine an initial PPR code predictive model;
calculating an initial scoring matrix for the initial PPR code predictive model;
updating the initial PPR code predictive model by performing an iterative expectation-maximization procedure until a best match of a target sequence to a PPR protein does not change any more, indicating a match between the PPR protein and target sequence, wherein the target sequence comprises an editing site; and
determining the presence of a DYW:JP domain in the PPR protein corresponding to the editing site, wherein the presence of the DYW:JP domain indicates the editing site is a site for U-to-C editing.

16. A computational method for predicting whether an editing site is a site for C-to-U editing, the method comprising:
receiving sequencing data representing PPR editing site in the organism and PPR proteins expressed in the organism;
estimating a background base composition for a PPR code;
assigning an initial nucleotide base preference for each PPR codon of the PPR proteins to determine an initial PPR code predictive model;
calculating an initial scoring matrix for the initial PPR code predictive model;
updating the initial PPR code predictive model by performing an iterative expectation-maximization procedure until a best match of a target sequence to a PPR protein does not change any more, indicating a match between the PPR protein and target sequence, wherein the target sequence comprises an editing site; and
determining the absence of a DYW:JP domain in the PPR protein corresponding to the editing site, wherein the absence of the DYW:JP domain indicates the editing site is a site for C-to-U editing.

17. The computational method of claim 1, wherein the sequence data represents all PPR editing sites in the whole genome of the organism and every PPR protein expressed in the organism.

18. The computational method of claim 12, wherein the sequence data represents all PPR editing sites in the whole genome of the organism and every PPR protein expressed in the organism.

19. The computational method of claim 16, wherein the sequence data represents all PPR editing sites in the whole genome of the organism and every PPR protein expressed in the organism.

20. The computational method of claim 17, wherein the sequence data represents all PPR editing sites in the whole genome of the organism and every PPR protein expressed in the organism.
